(19) United States
(12) Patent Application Publication
(10) Pub. No.: US 2020/0347394 A1
(43) Pub. Date: Nov. 5, 2020

(54) GENES AND GENE COMBINATIONS FOR ENHANCED CROPS

(71) Applicant: YIELD10 BIOSCIENCE, INC., Woburn, MA (US)

(72) Inventors: Madana M.R. AMBAVARAM, Norwood, MA (US); Jihong TANG, West Roxbury, MA (US); Mariya SOMLEVA, Woburn, MA (US); Kieran RYAN, Sharon, MA (US); Oliver P. PEOPLES, Arlington, MA (US); Kristi D. SNELL, Belmont, MA (US)

(21) Appl. No.: 16/760,639

(22) PCT Filed: Nov. 2, 2018
(86) PCT No.: PCT/US2018/058840

§ 371 Date: Apr. 30, 2020

Related U.S. Application Data
(60) Provisional application No. 62/580,959, filed on Nov. 2, 2017.

(51) Int. Cl. C12N 15/82; C12N 15/113; C12N 9/22

(52) U.S. Cl. CPC C12N 15/8216; C12N 15/113; C12N 2800/80; C12N 15/8247; C12N 2310/20; C12N 9/22

(57) ABSTRACT

Plant transcription factors and genes encoding the transcription factors are disclosed. Methods to enhance characteristics in a plant by downregulating the genes encoding the transcription factors also are disclosed. The enhanced characteristics can include higher photosynthesis rates, reduced photorespiration rates, higher biomass yield or content, higher seed yield, improved harvest index, higher oil content, improved nutritional composition, improved nitrogen use efficiency, drought resistance, flood resistance, disease resistance, faster seed germination and plant emergence, improved seedling vigor, salt tolerance, higher CO₂ assimilation rate, and lower transpiration rate. Modified plants in which the genes encoding the transcription factors are downregulated also are disclosed. Compositions of the invention comprise polynucleotide sequences, polypeptide sequences, variants, orthologs, and fragments thereof. Methods comprise introducing into plants systems that reduce or eliminate the expression of transcription factors. Methods and compositions also provide plants with enhanced seed yield and/or seed oil content.

### FIELD OF THE INVENTION

The present invention relates generally to gene targets, genome editing materials and methods for controlling the expression of those gene targets alone or in combinations and more particularly, to plants having reduced expression of those gene targets such that they have improved performance in soil as compared to the same plant having normal expression of those genes.

### BACKGROUND OF THE INVENTION

The world faces a major challenge in the next 35 years to meet the increased demands for food production to feed a growing global population, which is expected to reach 9 billion by the year 2050. Food output will need to be increased by up to 60% in view of the growing population.

Major agricultural crops include food crops, such as maize, wheat, oats, barley, soybean, millet, sorghum, potato, pulse, bean, tomato, rice, cassava, sugar beets, and potatoes, among others, forage crop plants, such as hay, alfalfa, and silage corn, among others, and oilseed crops, such as camelina, Brassica species (e.g. B. napus (canola), B. rapa, B. juncea, and B. carinata), crambe, soybean, sunflower, safflower, oil palm, flax, and cotton, among others. Crop yield can also be reduced as a consequence of weather patterns, such as heat waves, freezing temperatures, drought or flooding conditions in a particular growing season. With intensive farming practices crop pests or diseases can also reduce yield.

During the late 1980's and early 1990's genetic engineering or transgenic plants were used for the first time to develop crops which are herbicide tolerant and/or pest or disease resistant by introducing genes from the most readily available source at the time, microorganisms, to impart these new functionalities. Unfortunately, “transgenic plants” or “GMO crops” or “biotech traits” are not widely accepted in a number of different jurisdictions and are subject to regulatory approval processes which are very time consuming and prohibitively expensive. The current regulatory framework for transgenic plants results in significant costs (˜$136 million per trait; McDougall, P. 2011, The cost and time involved in the discovery, development, and authorization of a new plant biotechnology derived trait. Crop Life International, https://croplife.org/wp-content/uploads/pdf_files/Getting-a-Biotech-Crop-to-Market-Phillips-McDougall-Study.pdf) and lengthy product development timelines that limit the number of technologies that are brought to market. These risks have severely impaired private investment and the adoption of innovation in this crucial sector. Recent advances in genome editing technologies provide an opportunity to precisely remove or inactivate specific plant genes or to alter their expression by modifying their promoter sequences to improve plant performance (Belhaj, K. 2013, Plant Methods, 9, 39; Khandagale & Nadal, 2016, Plant Biotechnol Rep, 10, 327). More importantly genome editing enables the ability to do this with combinations of gene targets either sequentially or simultaneously. The challenge then is to identify which genes to modify by genome editing to improve plant performance.

Plant scientists have been able to identify the ancient ancestors of modern major agricultural crops and have begun to map the key genetic changes that have taken place through the crop domestication process resulting in these crops. Many of these changes have resulted from the modification of the activity of key plant regulator genes or transcription factors. A classic example is the domestication of modern corn from the ancient plant Teosinte (Matsuoka, Y. et al., 2002, PNAS, 99, 6080-6084). Today we know that the modern corn genome contains around 39,000 genes and about 2,500 of these are transcription factors (Lin, et. al., 2014, BMC Genomics, 15, 818-820). Based on the teosinte-domestication-to-corn analogy it might seem reasonable to assume that by altering the activity of a relatively small number of transcription factors in plants used for food and feed production, significant improvements in crop performance could be achieved. For example it may be possible to improve the performance of corn substantially using genome editing tools to modify the expression of transcription factor genes. However, simple analysis explains why it is not feasible to consider testing these one by one and/or in all combinations. To test all two-transcription-factor-gene combinations would require over 3.3 million individual experiments.

Clearly there is a need to develop systems and approaches to identifying small number of transcription factors whose expression can be modified alone or in combinations to improve crop performance.

### BRIEF SUMMARY OF THE INVENTION

It is an object of the current invention to provide methods, materials and plants useful for identifying a number of transcription factor genes, and transcription factor gene combinations, as targets for modification to improve crop performance. It is a further objective of this invention to provide a set of specific transcription factor genes for each of corn, soybean and canola as well as their orthologs in other plant species, in particular alfalfa, sorghum, rice, sugar beets and wheat as well as the methods, DNA and RNA sequences for modifying or editing these transcription factor genes and transcription factor gene combinations to modulate their expression or activity and improve the performance of plants. It is a further objective of this invention to provide crops, including corn, soybean, canola, alfalfa, sorghum, rice, sugar beets and wheat, which have been modified according to this invention and which have improved performance characteristics in the field as compared to the same crops before they were modified as disclosed herein.

A method for modifying a plant is provided. The method comprises downregulating one or more of:

(a) at least one polynucleotide sequence comprising one or more of SEQ ID NOs: 1, 2, 3, 4, 5, 6, 7, 8, 9, 10, 11, 12, 13, 14, 15, 16, 17, 18, 19, 20, 21, 22, 23, 24, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 287, 288, 544, 545, 722-741, or 762;

(b) at least one polynucleotide sequence comprising a sequence having at least 30%, 35%, 40%, 45%, 50%, 55%, 60%, 65%, 70%, 75%, 80%, 85%, 90%, 95% or higher sequence identity to at least one polynucleotide sequence set forth in (a);

(c) at least one polypeptide sequence encoded by at least one polynucleotide sequence set forth in (a) or (b);

(d) at least one polypeptide sequence comprising one or more of SEQ ID NOs: 289-542, 546, 547, 742-761, or 763; or

(e) at least one polypeptide sequence having at least 30%, 35%, 40%, 45%, 50%, 55%, 60%, 65%, 70%, 75%, 80%, 85%, 90%, 95% or higher sequence identity to at least one polypeptide sequence set forth in (d).

In some embodiments, the method further comprises growing the modified plant under conditions whereby the modified plant exhibits one or more enhanced characteristics as compared to a control plant grown under similar conditions.

In some embodiments, polynucleotide sequences comprising each of SEQ ID NOs: 22, 3, 9, 10, 14, 18, and 24 are downregulated. In some embodiments, polynucleotide sequences comprising each of SEQ ID NOs: 1, 3, 7, and 22 are downregulated. In some embodiments, polynucleotide sequences comprising each of SEQ ID NOs: 22, 28, 29, 30, 31, 32, 33, 34, 282, and 285 are downregulated. In some embodiments, a polynucleotide sequence comprising SEQ ID NO: 22 is downregulated in combination with downregulation of at least one polynucleotide sequence comprising one or more of SEQ ID NOs: 1, 2, 3, 4, 5, 6, 7, 8, 9, 10, 11, 12, 13, 14, 15, 16, 17, 18, 19, 20, 21, 23, or 24. In some embodiments, a polynucleotide sequence comprising SEQ ID NO: 22 is downregulated in combination with downregulation of at least one polynucleotide sequence comprising one or more of SEQ ID NOs: 2, 9, 10, or 18.

In some embodiments, the at least one polynucleotide sequence that is downregulated exhibits at least one of (i) a change in expression as compared to that of a control plant, or (ii) at least a two-fold change in expression as compared to that of a control plant. In some of these embodiments, the at least one polynucleotide sequence that is downregulated has been downregulated by overexpression of one or more global transcription factors selected from STR1, BMY, or STIF1.

In some embodiments, the at least one polynucleotide sequence that is downregulated has been downregulated by one or more of gene inactivation, deletion, insertion and/or substitution of one or more nucleotides, site-specific mutagenesis, chemical mutagenesis, targeting induced local lesions in genomes (TILLING), knock-out techniques, gene editing techniques using CRISPR nuclease selected from Cas nuclease, Cas9 nuclease, CasX nuclease, CasY nuclease, a Cpf1 nuclease, a C2c1 nuclease, a C2c2 nuclease (Cas13a nuclease), or a C2c3 nuclease, NgAgo nuclease, TALEN or ZFN techniques, or gene silencing induced by RNA interference. In some of these embodiments, the polynucleotide sequence that is downregulated has been downregulated by targeting at least one guide polynucleotide to one or more target sites selected from a promoter, a terminator or a coding sequence of the at least one polynucleotide sequence.

In some embodiments, the modified plant is one or more of a crop plant, a model plant, a monocotyledonous plant, a dicotyledonous plant, a plant with C3 photosynthesis, a plant with C4 photosynthesis, an annual plant, a perennial plant, a switchgrass plant, a maize plant, or a sugarcane plant. In some embodiments, the modified plant is soybean, canola, Medicago truncatula, alfalfa, sorghum, rice, wheat or Camelina.

In some embodiments, the modified plant exhibits one or more enhanced characteristics selected from higher photosynthesis rates, reduced photorespiration rates, higher biomass yield or content, higher seed yield, improved harvest index, higher seed oil content, improved nutritional composition, improved nitrogen use efficiency, drought resistance, flood resistance, disease resistance, faster seed germination and plant emergence, improved seedling vigor, salt tolerance, higher CO₂ assimilation rate, or lower transpiration rate. In some of these embodiments, the modified plant exhibits an increase in seed oil content or seed yield as compared to a control plant. In some of these embodiments, the seed oil content or seed yield of the modified plant is increased by 10%, 20%, 30%, 40%, 50%, 60%, 70%, 80%, 90%, 100% or higher relative to a control plant.

A modified plant also is disclosed. The modified plant comprises: (a) at least one polynucleotide sequence comprising one or more of SEQ ID NOs: 1, 2, 3, 4, 5, 6, 7, 8, 9, 10, 11, 12, 13, 14, 15, 16, 17, 18, 19, 20, 21, 22, 23, 24, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 287, 288, 544, 545, 722-741, or 762;

(b) at least one polynucleotide sequence comprising a sequence having at least 30%, 35%, 40%, 45%, 50%, 55%, 60%, 65%, 70%, 75%, 80%, 85%, 90%, 95% or higher sequence identity to at least one polynucleotide sequence set forth in (a);

(c) at least one polypeptide sequence encoded by at least one polynucleotide sequence set forth in (a) or (b);

(d) at least one polypeptide sequence comprising one or more of SEQ ID NOs: 289-542, 546, 547, 742-761, or 763; or

(e) at least one polypeptide sequence having at least 30%, 35%, 40%, 45%, 50%, 55%, 60%, 65%, 70%, 75%, 80%, 85%, 90%, 95% or higher sequence identity to at least one polypeptide sequence set forth in (d),

wherein the least one polynucleotide sequence set forth in (a) or (b) or the at least one polypeptide sequence set forth in (c), (d), or (e) is downregulated, either alone or in combination with at least another polynucleotide sequence set forth in (a) or (b) or at least another polypeptide sequence set forth in (c), (d), or (e).

In some embodiments, at least two polynucleotide sequences set forth in (a) or (b) or at least two polypeptide sequences set forth in (c), (d), or (e) are downregulated.

In some embodiments, the at least one polynucleotide sequence that is downregulated exhibits at least one of (i) a change in expression as compared to that of a control plant, or (ii) at least a two-fold change in expression as compared to that of a control plant.

In some embodiments, the modified plant is one or more of a crop plant, a model plant, a monocotyledonous plant, a dicotyledonous plant, a plant with C3 photosynthesis, a plant with C4 photosynthesis, an annual plant, a perennial plant, a switchgrass plant, a maize plant, or a sugarcane plant. In some embodiments, the modified plant is soybean, canola, Medicago truncatula, alfalfa, sorghum, rice, wheat or Camelina.

In some embodiments, the modified plant exhibits one or more enhanced characteristics selected from higher photosynthesis rates, reduced photorespiration rates, higher biomass yield or content, higher seed yield, improved harvest index, higher seed oil content, improved nutritional composition, improved nitrogen use efficiency, drought resistance, flood resistance, disease resistance, faster seed germination and plant emergence, improved seedling vigor, salt tolerance, higher CO₂ assimilation rate, or lower transpiration rate. In some of these embodiments, the modified plant exhibits an increase in seed oil content or seed yield as compared to a control plant. Also in some of these embodiments, the seed oil content or seed yield of the modified plant is increased by 10%, 20%, 30%, 40%, 50%, 60%, 70%, 80%, 90%, 100% or higher relative to a control plant.

In some embodiments, the at least one polynucleotide sequence that is downregulated has been downregulated by one or more of gene inactivation, deletion, insertion and/or substitution of one or more nucleotides, site-specific mutagenesis, chemical mutagenesis, targeting induced local lesions in genomes (TILLING), knock-out techniques, gene editing techniques using CRISPR nuclease selected from Cas nuclease, Cas9 nuclease, CasX nuclease, CasY nuclease, a Cpf1 nuclease, a C2c1 nuclease, a C2c2 nuclease (Cas13a nuclease), or a C2c3 nuclease, NgAgo nuclease, TALEN or ZFN techniques, or gene silencing induced by RNA interference. In some of these embodiments, the polynucleotide sequence that is downregulated has been downregulated by targeting at least one guide polynucleotide to one or more target sites selected from a promoter, a terminator or a coding sequence of the at least one polynucleotide sequence.

A recombinant nucleic acid molecule also is disclosed. The recombinant nucleic acid molecule comprises:

(a) at least one polynucleotide sequence comprising one or more of SEQ ID NOs: 1-24, 35-117, 141-285, 287, 288, 544, 545, 722-741, or 762;

(b) at least one polynucleotide sequence comprising a sequence having at least 30%, 35%, 40%, 45%, 50%, 55%, 60%, 65%, 70%, 75%, 80%, 85%, 90%, 95% or higher sequence identity to at least one polynucleotide sequence set forth in (a); or

(c) a fragment of at least one polynucleotide sequence set forth in (a) or (b) that regulates gene expression.

A recombinant polypeptide molecule also is disclosed. The recombinant polypeptide molecule comprises:

(a) at least one polypeptide sequence comprising one or more of SEQ ID NOs: 289-542, 546, 547, 742-761, or 763;

(b) at least one polypeptide sequence comprising a sequence having at least 30% 35%, 40%, 45%, 50%, 55%, 60%, 65%, 70%, 75%, 80%, 85%, 90%, 95% or higher sequence identity to at least one polypeptide sequence set forth in (a); or

(c) a fragment of at least one polypeptide sequence of (a) or (b) that regulates gene expression.

A DNA construct also is disclosed. The DNA construct comprises:

(a) an expression cassette containing a polynucleotide sequence encoding a CRISPR nuclease;

(b) DNA encoding at least one guide RNA targeting the 5′ upstream region, promoter, terminator or coding sequence of one or more of SEQ ID NOs: 1-24, 35-117, 141-285, 287, 288, 544, 545, 722-741, or 762 or a polynucleotide sequence having at least 30% 35%, 40%, 45%, 50%, 55%, 60%, 65%, 70%, 75%, 80%, 85%, 90%, 95% or higher sequence identity to one or more of SEQ ID NOs: 1-24, 35-117, 141-285, 287, 288, 544, 545, 722-741, or 762; and

(c) an expression cassette for a selectable marker.

In some embodiments, the DNA encoding the at least one guide RNA is capable of downregulating a polynucleotide sequence comprising one or more of SEQ ID NOs: 1-24, 35-117, 141-285, 287, 288, 544, 545, 722-741, or 762, thereby producing enhanced characteristics in a plant selected from higher photosynthesis rates, reduced photorespiration rates, higher biomass yield or content, higher seed yield, improved harvest index, higher seed oil content, improved nutritional composition, improved nitrogen use efficiency, drought resistance, flood resistance, disease resistance, faster seed germination and plant emergence, improved seedling vigor, salt tolerance, higher CO₂ assimilation rate, or lower transpiration rate.

A modified plant transformed with the DNA construct also is provided. In some embodiments, at least one polynucleotide sequence comprising one or more of SEQ ID NOs: 1-24, 35-117, 141-285, 287, 288, 544, 545, 722-741, or 762 is downregulated. In some of these embodiments, the at least one polynucleotide sequence that is downregulated exhibits at least one of (i) a change in expression as compared to that of a control plant, or (ii) at least a two-fold change in expression as compared to that of a control plant. In some examples of these embodiments, the modified plant is one or more of a crop plant, a model plant, a monocotyledonous plant, a dicotyledonous plant, a plant with C3 photosynthesis, a plant with C4 photosynthesis, an annual plant, a perennial plant, a switchgrass plant, a maize plant, or a sugarcane plant. Also in some examples of these embodiments, the modified plant is soybean, canola, Medicago truncatula, alfalfa, sorghum, rice, wheat or Camelina. Also in some examples of these embodiments, the modified plant exhibits one or more enhanced characteristics selected from higher photosynthesis rates, reduced photorespiration rates, higher biomass yield or content, higher seed yield, improved harvest index, higher seed oil content, improved nutritional composition, improved nitrogen use efficiency, drought resistance, flood resistance, disease resistance, faster seed germination and plant emergence, improved seedling vigor, salt tolerance, higher CO₂ assimilation rate, or lower transpiration rate. In some of these examples, the modified plant exhibits an increase in seed oil content or seed yield as compared to a control plant. In some of these examples, the seed oil content or seed yield of the modified plant is increased by 10%, 20%, 30%, 40%, 50%, 60%, 70%, 80%, 90%, 100% or higher relative to a control plant.

A modified seed comprising the DNA construct also is provided.

A method of modifying a plant cell also is disclosed. The method comprises:

(a) expressing one or more site-specific nucleases in a plant cell, wherein the one or more nucleases target and cleave chromosomal DNA of one or more endogenous genes, their promoters, their 5′UTRs, and/or their polyadenylation sequences, and wherein the one or more endogenous genes comprise one or more of SEQ ID NOs: 1-24, 35-117, 141-285, 287, 288, 544, 545, 722-741, or 762;

(b) integrating one or more exogenous sequences into the one or more endogenous genes, their promoters, their 5′UTRs, and/or their polyadenylation sequences within the genome of the plant cell, wherein the one or more endogenous genes, their promoters, their 5′UTRs, and/or their polyadenylation sequences are modified such that the one or more endogenous genes do not express their corresponding endogenous gene product(s); and

(c) selecting plant cells that exhibit enhanced characteristics from among the plant cells in which the one or more exogenous sequences have been integrated.

In some embodiments, the one or more exogenous sequences are selected from a donor polynucleotide, a transgene, or a combination thereof. In some embodiments, the one or more exogenous sequences encode a transgene and/or are expressed to produce an RNA molecule. In some embodiments, the one or more exogenous sequences comprise a multiplex of gene edits made in the one or more endogenous genes, their promoters, their 5′UTRs, and/or their polyadenylation sequences.

In some embodiments, the integrating of the one or more exogenous sequences occurs by homologous recombination or non-homologous end joining.

In some embodiments, the one or more site-specific nucleases are selected from a zinc finger nuclease, a TAL effector domain nuclease, a homing endonuclease, or a CRISPR/Cas or a CRISPR/Cpf1 single guide RNA nuclease.

In some embodiments, the one or more endogenous genes comprising one or more of SEQ ID NOs: 1-24, 35-117, 141-285, 287, 288, 544, 545, 722-741, or 762 is downregulated. In some of these embodiments, the one or more endogenous genes that are downregulated exhibit at least one of (i) a change in expression as compared to that of a control plant, or (ii) at least a two-fold change in expression as compared to that of a control plant.

In some embodiments, the modified plant cell is a cell of one or more of a crop plant, a model plant, a monocotyledonous plant, a dicotyledonous plant, a plant with C3 photosynthesis, a plant with C4 photosynthesis, an annual plant, a perennial plant, a switchgrass plant, a maize plant, or a sugarcane plant. In some embodiments, the modified plant cell is a cell of soybean, canola, Medicago truncatula, alfalfa, sorghum, rice, wheat or Camelina.

In some embodiments, the method further comprises cultivating the modified plant cell to obtain a modified plant that exhibits one or more enhanced characteristics selected from higher photosynthesis rates, reduced photorespiration rates, higher biomass yield or content, higher seed yield, improved harvest index, higher seed oil content, improved nutritional composition, improved nitrogen use efficiency, drought resistance, flood resistance, disease resistance, faster seed germination and plant emergence, improved seedling vigor, salt tolerance, higher CO₂ assimilation rate, or lower transpiration rate. In some of these embodiments, the modified plant exhibits an increase in seed oil content or seed yield as compared to a control plant. In some examples of these embodiments, the seed oil content or seed yield of the modified plant is increased by 10%, 20%, 30%, 40%, 50%, 60%, 70%, 80%, 90%, 100% or higher relative to a control plant.

Accordingly, provided are modified plants transformed with the DNA construct, as well as modified seeds and progeny comprising the DNA construct. In some examples, the modified plants have at least one gene downregulated, which exhibits at least two-fold change in expression to that of a control plant.

Surprisingly, SEQ ID NO: 22 stands out as the sole downstream transcription factor that was downregulated by more than two-fold by all three global transcription factors STR1, STIF1 and BMY1, indicative of a good gene target as a negative regulator, as demonstrated in Example 2.

Plants of interest include a crop plant, a model plant, a monocotyledonous plant, a dicotyledonous plant, a plant with C3 photosynthesis, a plant with C4 photosynthesis, an annual plant, a perennial plant, a switchgrass plant, a maize plant, or a sugarcane plant. More preferably, the crop is selected from soybean, canola, alfalfa, sorghum, rice, wheat and Camelina.

The modified crop exhibits one or more enhanced characteristics selected from higher photosynthesis rates, reduced photorespiration rates, higher biomass yield or content, higher seed yield, improved harvest index, higher oil content, improved nutritional composition, drought resistance, flood resistance, disease resistance, faster seed germination and plant emergence, improved seedling vigor, higher CO₂ assimilation rate, and lower transpiration rate. In particular, the modified plant with the reduced expression of SEQ ID NO: 22 exhibits an increase in seed oil content or seed yield compared to that of a control plant. Preferably, the yield is increased by 10%, 20%, 30%, 40%, 50%, 60%, 70%, 80%, 90%, 100% or higher relative to a control plant.

### BRIEF DESCRIPTION OF THE DRAWINGS

FIG. 1 illustrates a Venn diagram that shows the number of downstream transcription factors (dTFs) that are down regulated by the global regulatory genes STR1, STF1, and BMY1 compared to wild-type controls. Only those dTFs where the log 2 fold change in expression is ≤−1 (downregulated by 2 fold or more) are shown. Intersecting circles show dTFs that are down regulated by two or more global regulatory genes. Only one dTF, dTF22, is down regulated by all three global regulatory genes by more than 2-fold.

FIG. 2 illustrates the plasmid map of binary vector pMBXS1032 for expression of dTF22 in monocots. The dTF22 gene is expressed from the maize cab-m5 light inducible promoter of the chlorophyll a/b/-binding protein fused to the intron from the maize heat shock protein 70 (hsp70).

FIG. 3 illustrates an amino acid alignment of the dTF22 sequence from switchgrass (Panicum virgatum) genotype YTEN(II56) and the dTF22 sequence as found in the sequenced switchgrass genotype AP13 (Phytozome). There are five amino acid differences in the amino acid sequences of the two proteins.

FIG. 4 illustrates biomass production in dTF22 overexpressing lines. The bars represent the average value of measurements of 3 plants per line as % to the wild type control. The control values (mean±SD, n=4) are as follows: 38.74±8.59 g dry weight (DW) total biomass, 13.72±1.32 g DW leaf biomass, 25.02±7.60 g DW stem biomass (leaf sheaths, nodes, internodes and panicles), and 28.00±3.74 total number of tillers.

FIG. 5 illustrates the phenotype of plants from the dTF22 overexpressing line 17, the smallest of the plants isolated. (A) Plants 1 month after transfer to soil. (B) Plants grown under greenhouse conditions for 3 months. Note the lack of normally developed stems. The plants did not form reproductive tillers by the end of the growth period of 4 months. WT, a wild type control plant; 1-3, transgenic plants of dTF22 line 17.

FIG. 6 illustrates genetic components at different stages of the Cas enzyme mediated genome editing process using the Cas9 enzyme as an example. Delivery of the genetic components can be achieved in multiple ways. Genetic transformation of the expression construct depicted in (A) into a plant cell will produce the single guide RNA (sgRNA) molecule in (B) that will complex with Cas9 (that is delivered separately through genetic transformation or other means) and achieve the structure depicted in (C) to promote cleavage of the target DNA. Alternatively, the sgRNA (B) can be synthesized in vitro and introduced into cells, often in the form of Ribonucleoprotein complexes (RNPs) that contain Cas9 protein to produce the structure depicted in (C) to promote cleavage of the target DNA. When using plant transformation techniques, an expression cassette (A) containing DNA encoding a sgRNA is used. This is composed of a promoter, often a plant RNA polymerase III promoter, DNA encoding a guide target sequence, DNA encoding a guide RNA scaffold (gRNA Sc), and a poly T-termination signal. The sequence of the guide target sequence is often identical to the target DNA to be cut, however several mismatches, depending on their position in the guide target sequence can be tolerated and still achieve double stranded DNA cleavage. Transcription of the expression cassette in (A) produces a sgRNA (B) which forms a complex with the Cas enzyme (C). The guide target sequence of the sgRNA pairs with the complementary DNA sequence to be mutated (C) that is adjacent to 3′ PAM sequence and double stranded DNA cleavage occurs. When using the Cas9 enzyme for cleavage, all guide target sequences are typically ˜20-nucleotides adjacent to a 3′ PAM sequence of (NGG) to initiate cleavage by the Cas9 enzyme. When using the CpfI enzyme for cleavage, guide target sequences are typically ˜23 nucleotides adjacent to a 5′ PAM sequence that varies with the specific enzyme. PAM sequences for select CpfI enzymes including engineered variants are shown in Table 17.

FIG. 7 illustrates the strategy for editing regions of a gene or its promoter using CRISPR. (A) At least five guide target sequences (FIG. 6, abbreviated Guide # x) are designed to target several regions spanning the promoter, 5′ untranslated regions (5′ UTR), and coding sequence of the gene. The general numbering strategy used for guide target sequences is as follows. The sequence of the 5′UTR of the gene of interest plus an additional 1000 bp was analyzed for guide target sequences to target portions of the promoter region for mutation or excision. Since the length of the 5′ UTR varies for each gene, x denotes the size of the known or predicted UTR. Position #(1000+x) is the base directly in front of the ATG at the start of the coding sequence. In most examples, three guide target sequences were designed in the promoter region and at least two within the coding sequence of the gene. (B) All guide target sequences can be used to form sgRNAs, as described in FIG. 6, that can be used individually to create simple INDELS (insertion or deletion of a small number of bases). In the coding region, this may create a frameshift which will truncate the protein. In the promoter region, this may modify the strength of the promoter or, in some regions, inactivate the promoter. Pairs of guide target sequences can be used to form sgRNAs that can be used to excise regions of DNA. Guide target sequences 1 and 2 or 1 and 3 can be selected to excise the indicated regions of the promoter. Guide target sequences #4 and #5 are designed to excise a substantial portion of the gene to inactivate the gene, including introns where present. n designates the number of intron/exon regions between guide target sequences #4 and #5 which varies with each gene. To excise both regions of the promoter and the coding sequence, guide target sequences 3 and 5 can be used. (C) Example deletion strategy using rice dTF22 gene (Gene ID LOC_Os03g41330, SEQ ID NO: 27) with a 160 bp 5′ UTR (x=160) and another 1000 bp of sequence upstream of the 5′UTR. The dTF22 gene contains 2 exons (n=1).

FIG. 8 illustrates the plasmid map of binary construct pYTEN-24 for Cas9 mediated genome editing of the coding sequence of the rice dTF22 gene using guide target sequence #4 (Table 2). The construct contains the 2×35S promoter driving the expression of the Cas9 gene which has been codon optimized for rice. The gene encoding Cas9 is flanked by nuclear localization sequences (NLS) to ensure delivery into nuclei. The rice codon-optimized Streptococcus pyrogenes Cas9 and NLS sequence were synthesized using sequences described by Shan et al., 2013, Nat Biotechnol, 3, 686-688. The CaMV terminator sequence is downstream of the gene encoding Cas9. The rice U6 promoter drives the expression of DNA guide target sequence #4, targeted to the rice dTF22 coding sequence (FIG. 7C, Table 2) in the rice genome, and DNA encoding the guide RNA scaffold producing a functional single guide RNA (sgRNA). A poly T-termination signal is located downstream of the guide target sequence and the DNA fragment encoding the guide RNA scaffold. An expression cassette for selection of transgenic plants for hygromycin resistance contains the CaMV35S promoter, an hsp70 intron, a hpt1 gene encoding hygromycin phosphotransferase containing an intron from the bean catalase-1 gene (CAT-1 intron), and a CaMV35S polyA termination sequence.

FIG. 9 illustrates the plasmid map of binary construct pMBXS1223 for Cas9 mediated genome editing of the coding sequence of the rice dTF22 gene using DNA guide target sequences #6 and 7 (Table 3). The construct contains the 2×35S promoter driving the expression of the Cas9 gene which has been codon optimized for rice. The gene encoding Cas9 is flanked by nuclear localization sequences (NLS) to ensure delivery into nuclei. The rice codon-optimized Streptococcus pyrogenes Cas9 and NLS sequence were synthesized using sequences described by Shan et al., 2013, Nat Biotechnol, 3, 686-688. The CaMV terminator sequence is downstream of the gene encoding Cas9. An expression cassette for guide target sequence #6 (FIG. 7C, Table 3) contains the rice U6 promoter, guide target sequence #6 targeted to the rice dTF22 coding sequence in the rice genome, DNA encoding the guide RNA scaffold, and a poly T-termination sequence. A second expression cassette contains the rice U6 promoter, guide target sequence #7 (FIG. 7C, Table 3) targeted to the rice dTF22 coding sequence in the rice genome, a DNA fragment encoding the guide RNA scaffold, and a poly T-termination sequence. An expression cassette for selection of transgenic plants for hygromycin resistance contains the CaMV35S promoter, an hsp70 intron, a hpt1 gene encoding hygromycin phosphotransferase containing an intron from the bean catalase-1 gene (CAT-1 intron), and a CaMV35S polyA termination sequence.

FIG. 10 illustrates the types of mutations observed in rice plants transformed with the editing vector pMBXS1223. (A) Mutations observed with guide target sequence #7 (FIG. 7C, Table 3). (B) Mutations observed with guide target sequence #6 (FIG. 7C, Table 3). The boxed regions highlight the observed mutations. The underlined TGG sequence is the PAM site.

FIG. 11 illustrates the plasmid map of binary construct pYTEN-25 for Cas9 mediated multiplex genome editing of the coding sequences of the maize dTF10, dTF18, dTF22, and dTF60 genes using guide target sequence #4 (Table 9) for dTF10, dTF18, and dTF22 and a guide target sequence with the sequence 5′-CTGAAGCCGAACCAGCCTGG-3′ (SEQ ID NO: 697) for dTF60. The construct contains the 2×35S promoter driving the expression of a gene expressing Cas9 which has been codon optimized for rice. The gene encoding Cas9 is flanked by nuclear localization sequences (NLS) to ensure delivery into nuclei. The rice codon-optimized Streptococcus pyrogens Cas9 and NLS sequence were synthesized using sequences described by Shan et al., 2013, Nat Biotechnol, 3, 686-688. A poly T-termination sequence is downstream of the gene encoding Cas9. An expression cassette for each guide target sequence contains: the rice U6 promoter driving the expression of both the guide target sequence as well as DNA encoding its associated guide RNA scaffold, and a poly T-termination sequence. An expression cassette for selection of transgenic plants contains the CaMV35S promoter, an hsp70 intron, a hpt1 gene encoding hygromycin phosphotransferase containing an intron from the bean catalase-1 gene (CAT-1 intron), and a CaMV35S polyA sequence to provide hygromycin resistance to transgenic plants.

### DETAILED DESCRIPTION OF THE INVENTION

The following terms, unless otherwise indicated, shall be understood to have the following meanings:

As used herein we use the terms “crops” and “plants” interchangeably. “Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” or “recombinant expression construct”, which are used interchangeably, refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure. As used herein the term “coding sequence” refers to a DNA sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences. As used herein “gene” includes protein coding regions of the specific genes and the regulatory sequences both 5′ and 3′ which control the expression of the gene.

As used herein a “modified plant” refers to non-naturally occurring plants or crops engineered as described throughout herein.

As used herein a “control plant” means a plant that does not contain the recombinant DNA of the present disclosure that imparts an enhanced trait or altered phenotype. A control plant is used to identify and select a modified plant that has an enhanced trait or altered phenotype. For instance, a control plant can be a plant that has not been modified or has not been genome edited to express or to inhibit its endogenous gene product. A suitable control plant can be a non-transgenic plant of the parental line used to generate a transgenic plant, for example, a wild type plant devoid of a recombinant DNA. A suitable control plant can also be a transgenic plant that contains recombinant DNA that imparts other traits, for example, a transgenic plant having enhanced herbicide tolerance. A suitable control plant can in some cases be a progeny of a hemizygous transgenic plant line that does not contain the recombinant DNA, known as a negative segregant, or a negative isogenic line.

As used herein the terms “biomass yield” or “biomass content” refer to increase or decrease in the % dry weight in an amount greater than an otherwise identical plant, cultured under identical conditions, but lacking any corresponding modification, e.g., gene editing or the transgene in a control plant.

As used herein the terms “oil seed yield” or “seed oil content” refer to oil content of a seed as measured, e.g., expressed on the basis of seed dry weight.

As used herein, the terms “reduce activity,” “reduce expression,” “down-regulating,” or “downregulated” are used interchangeably and mean the activity of the transcription factor is reduced or lower than the expression of the same gene in the same plant species before the gene was modified as described herein. Downregulation should be understood to include a decrease in the level or activity of a target gene in a cell and/or substantially complete inhibition of a particular target polypeptide in a cell which normally expresses the target polypeptide. For instance, a 1%, 5%, 10%, 20%, 30%, 40%, 50%, 60%, 70%, 80%, 90%, 100%, 2-fold, 5-fold, 10-fold, 20-fold, 50-fold, 100-fold decrease in the level of activity of a target polypeptide in the cell. With respect to term “2-fold reduction”, “downregulated 2-fold” and 100% decrease is used interchangeably.

“Codon degeneracy” refers to divergence in the genetic code permitting variation of the nucleotide sequence without affecting the amino acid sequence of an encoded polypeptide. Accordingly, the instant invention relates to any nucleic acid fragment comprising a nucleotide sequence that encodes all or a substantial portion of the amino acid sequences set forth herein. The skilled artisan is well aware of the “codon-bias” exhibited by a specific host cell in usage of nucleotide codons to specify a given amino acid. Therefore, when synthesizing a nucleic acid fragment for increased expression in a host cell, it is desirable to design the nucleic acid fragment such that its frequency of codon usage approaches the frequency of preferred codon usage of the host cell.

As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity). When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percent sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percent sequence identity” means the value determined by comparing two aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percent sequence identity.

The term “plant” includes whole plant, mature plants, seeds, shoots and seedlings, and parts, propagation material, plant organ tissue, protoplasts, callus and other cultures, for example cell cultures, derived from plants belonging to the plant subkingdom Embryophyta, and all other species of groups of plant cells giving functional or structural units, also belonging to the plant subkingdom Embryophyta. The term “mature plants” refers to plants at any developmental stage beyond the seedling. The term “seedlings” refers to young, immature plants at an early developmental stage.

The modern corn genome contains around 39,000 thousand genes and about 2,500 of these are transcription factors (Lin, et. al., 2014, BMC Genomics, 15, 818-820). Herein we have focused on identifying a number of transcription factor genes which we have shown to function as negative regulators of plant growth and performance based on their expression being significantly reduced in engineered plants with higher photosynthesis, higher carbon flux through central metabolism and increased biomass production (WO2014100289 to Yield10 Bioscience) as described in detail in Example 1. The reduced expression of these specific genes in genetically engineered plants with higher photosynthesis, carbon flux through central metabolism and yield of fixed carbon in the form of biomass indicates that they function as negative controllers of plant growth and performance and hence are good targets for reducing their expression to improve crop performance.

Although the genes are identified in each crop by the sequence ID numbers for the structural gene in the Examples and Tables herein, it is well understood by those skilled in the art to identify the DNA sequences 5′ and 3′ to the structural gene to identify sequences controlling the expression of the transcription factor genes of interest in the specific crops of interest. It is also well known in the art that different crops may have different numbers of copies of each chromosome and hence may have more than one copy of each of the 24 transcription factor genes and there may be sequence differences in each copy of the gene in a particular crop species.

### PREFERRED EMBODIMENTS

The present disclosure relates to transcription factor genes in specific crop species whose expression or activity can be modulated to increase crop performance and crops having reduced expression of these transcription factor genes alone and in combinations which have improved performance compared to the same plants with normal expression levels of these genes. Also disclosed are specific transcription factor gene sequences, DNA sequences, RNA sequences and materials and methods for modifying plant cells and plants such that they have reduced expression of the transcription factor genes, methods for identifying plant cells and plants with reduced expression of the transcription factor genes and methods for producing fertile plants with reduced expression of the transcription factor genes wherein the modified plants have improved performance as compared to the same plants before they were modified to reduce the expression of these genes.

In various aspects, the present invention provides transcription factor genes useful for practicing the disclosed invention and include those that can function as negative controllers or feedback controllers in plants. Plants evolved over millennia simply to survive and reproduce before the involvement of humans to domesticate specific plants which we recognize today as the major food and feed crops. During the domestication process the intervention of humans either through agronomic practices or through crop breeding led to the “unnatural” selection of crops for specific purposes, for example corn for grain yield, and sorghum and alfalfa for forage applications. There is genetic evidence from the domestication of teosinte to corn (maize) that the downregulation or reduced expression of transcription factors was important in achieving the performance and grain yield of the modern crop. Transcription factors function to either increase the activity of specific metabolic pathways or gene regulatory networks in plants or to decrease them. Herein we have identified 24 transcription factors in crops which may act as negative controllers of key plant systems related to crop performance. It is well known in the field of metabolic engineering (synthetic biology) that a key to increasing the yield of a particular target product is to remove the negative control steps in the metabolic systems or pathways related to the target of interest. Without wishing to be bound by the theory, we believe one way to significantly improve the performance of crops is to identify and remove or down regulate key negative control points alone and in combinations in plant genes involved in gene regulation and metabolism. Herein we disclose 24 transcription factor genes which function as negative controllers in switchgrass and their equivalents or orthologs in major crop species whose reduced expression is important for improved performance as described in Example 1, Table 1. Also disclosed in Table 1 are combinations of these 24 transcription factor genes which function as negative controllers in switchgrass and their equivalents or orthologs in major crop species whose reduced expression is important for improved performance.

In one embodiment, 24 switchgrass transcription factors have been identified as functioning as negative controllers of plant production in transgenic switchgrass lines. These transcription factor genes and their orthologs and homologs in other crops are useful targets for modification to reduce their expression and improve plant performance. Other crops of interest for the disclosed invention include corn, soybean, canola, sorghum, rice, wheat and alfalfa and many other crops. The 24 switchgrass transcription factors include SEQ ID NO: 1 (Pavirv00029177m), SEQ ID NO: 2 (Pavirv00003507m), SEQ ID NO: 3 (AP13CTG12699_at), SEQ ID NO: 4 (Pavirv00024770m), SEQ ID NO: 5 (Pavirv00012672m), SEQ ID NO: 6 (Pavirv00006905m), SEQ ID NO: 7 (Pavirv00011545m), SEQ ID NO: 8 (Pavirv00039321m), SEQ ID NO: 9 (Pavirv00007251m), SEQ ID NO: 10 (AP13ITG41879_s_at), SEQ ID NO: 11 (Pavirv00007239m), SEQ ID NO: 12 (Pavirv00003464), SEQ ID NO: 13 (Pavirv00006072m), SEQ ID NO: 14 (Pavirv00000078m), SEQ ID NO: 15 (Pavirv00012008m), SEQ ID NO: 16 (AP13CTG14279ST_s_at), SEQ ID NO: 17 (Pavirv00053825m), SEQ ID NO: 18 (Pavirv00008285m), SEQ ID NO: 19 (Pavirv00010659m), SEQ ID NO: 20 (Pavirv00067953m), SEQ ID NO: 21 (Pavirv00005696m), SEQ ID NO: 22 (Pavirv00012971m), SEQ ID NO: 23 (Pavirv00056268m) and SEQ ID NO: 24 (Pavirv00036358m).

Isolated nucleic acid molecules for genes encoding enzymes, and variants thereof, are provided. Exemplary full-length nucleic acid sequences for genes encoding enzymes and the corresponding amino acid sequences are presented in Tables 1, 5, 6, 8, and 10-15. The nucleic acid sequence can be preferably greater than 80%, 85%, 90%, 95%, 98%, 99%, 99.9% or even higher identity to the wild-type gene.

In another embodiment, the nucleic acid molecule encodes a polypeptide having an amino acid sequence disclosed in the Table(s). Preferably, the nucleic acid molecule encodes a polypeptide sequence of at least 50%, 60, 70%, 80%, 85%, 90% or 95% identity to the amino acid sequences shown in the Table(s) and the identity can even more preferably be 96%, 97%, 98%, 99%, 99.9% or even higher.

According to another aspect, isolated polypeptides (including muteins, allelic variants, fragments, derivatives, and analogs) encoded by the nucleic acid molecules are provided. In one embodiment, the isolated polypeptide comprises the polypeptide sequence corresponding to a polypeptide sequence shown in the Table(s).

In an alternative embodiment, the isolated polypeptide comprises a polypeptide sequence at least 30%, 35%, 40%, 45%, 50%, 55%, 60%, 65%, 70%, 75%, 80%, 85%, 90%, 95% or higher sequence identity to the polypeptide sequences shown in the Table(s). Preferably the isolated polypeptide has at least 50%, 60, 70%, 80%, 85%, 90%, 95%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or even higher identity to a polypeptide SEQ ID NOs: 289-542, 546, 547, 742-761, or 763.

According to other embodiments, isolated polypeptides comprising a fragment of the above-described polypeptide sequences are provided. These fragments preferably include at least 20 contiguous amino acids, more preferably at least 25, 30, 35, 40, 45, 50, 60, 70, 80, 90, 100 or even more contiguous amino acids.

Nucleic acid molecules that hybridize under stringent conditions to the above-described nucleic acid molecules also are provided. As defined above, and as is well known in the art, stringent hybridizations are performed at about 25° C. below the thermal melting point (Tₘ) for the specific DNA hybrid under a particular set of conditions, where the Tₘ is the temperature at which 50% of the target sequence hybridizes to a perfectly matched probe. Stringent washing is performed at temperatures about 5° C. lower than the Tₘ for the specific DNA hybrid under a particular set of conditions.

Nucleic acid molecules comprising a fragment of any one of the above-described nucleic acid sequences are also provided. These fragments preferably contain at least 20 contiguous nucleotides. More preferably the fragments of the nucleic acid sequences contain at least 25, 30, 35, 40, 45, 50, 60, 70, 80, 90, 100 or even more contiguous nucleotides.

The different families of transcription factors found in crops are described for example by Lin, et. al., (2014, BMC Genomics, 15, 818-820).

SEQ ID NO: 1 encodes a transcription factor gene which contains a sequence-specific DNA binding domain with homology to proteins in the MYB family. This gene is predicted to be involved in the abscisic acid (ABA)-response and also interact with other downstream transcription factors for improvement of grain yield and stress tolerance by modification of cell cycle and/or photosynthesis pathways.

SEQ ID NO: 2 encodes a transcription factor gene which contains a sequence-specific DNA binding domain with homology to proteins in the MYB family. This gene is predicted to be involved in abscisic acid (ABA) signaling cascade to regulate stomatal movement and drought stress and disease resistance.

SEQ ID NO: 3 encodes a transcription factor gene which contains a sequence-specific DNA binding domain with homology to proteins in the MYB family. This gene is predicted to play a crucial role in the control of the cell cycle.

SEQ ID NO: 4 encodes a transcription factor gene which contains a sequence-specific DNA binding domain with homology to proteins in the bZIP family and is predicted to regulate processes including pathogen defense, light and stress signaling, seed maturation and flower development.

SEQ ID NO: 5 encodes a transcription factor gene which contains a sequence-specific DNA binding domain with homology to proteins in the G2-like family of transcription factors, which are members of the GARP superfamily of transcription factors. This gene is predicted to be involved in chloroplast development in both green and non-green tissues.

SEQ ID NO: 6 encodes a transcription factor gene which contains a sequence-specific DNA binding domain with homology to proteins in the CO-like family. CO (CONSTANS) genes act between the circadian clock and genes controlling meristem identity which also suggests a possible role in late or delayed flowering.

SEQ ID NO: 7 encodes a transcription factor gene which contains a sequence-specific DNA binding domain with homology to proteins in the bZIP family with a predicted role in regulating a number of processes including pathogen defense, light and stress signaling, seed maturation and flower development.

SEQ ID NO: 8 encodes a transcription factor gene which contains a sequence-specific DNA binding domain with homology to proteins in the ERF family. This transcription factor family includes dehydration-responsive element-binding proteins (DREBs), which activate the expression of abiotic stress-responsive genes and the transcriptional regulation of a variety of biological processes related to growth and development.

SEQ ID NO: 9 encodes a transcription factor gene which contains a sequence-specific DNA binding domain with homology to proteins in the HD-ZIP family. The homeodomain-leucine zipper (HD-Zip) proteins are transcription factors unique to plants and this protein is predicted to be involved in light response, shade avoidance and auxin signaling.

SEQ ID NO: 10 encodes a transcription factor gene which contains a sequence-specific DNA binding domain with homology to proteins in the HSF family. The Heat stress transcription factor (HSF) gene is predicted to be involved in abiotic stresses such as high temperature, salinity, and drought which adversely affect the survival, growth, and reproduction of plants.

SEQ ID NO: 11 encodes a transcription factor gene which contains a sequence-specific DNA binding domain with homology to proteins in the bZIP family. This gene is predicted to be involved in many central developmental and physiological processes including photomorphogenesis, leaf and seed formation, energy homeostasis, and abiotic and biotic stress responses.

SEQ ID NO: 12 encodes a transcription factor gene which contains a sequence-specific DNA binding domain with homology to proteins in the family G2-like family and has a predicted role as a transcriptional regulator of chloroplast development.

SEQ ID NO: 13 encodes a transcription factor gene which contains a sequence-specific DNA binding domain with homology to proteins in the ERF family. This gene is predicted to be involved in transcriptional regulation of a variety of biological processes related to growth and development, as well as cold and freezing stress tolerance.

SEQ ID NO: 14 encodes a transcription factor gene which contains a sequence-specific DNA binding domain with homology to proteins in the MYB family and its expression correlates with abiotic (drought, cold and salinity) stress responsive genes.

SEQ ID NO: 15 encodes a transcription factor gene which contains a sequence-specific DNA binding domain with homology to proteins in the C2H2 family. The protein encoded by this gene belongs to the family of C2H2-type zinc-finger proteins. It functions as a transcriptional regulator that activates genes involved in primary metabolic processes.

SEQ ID NO: 16 encodes a transcription factor gene which contains a sequence-specific DNA binding domain with homology to proteins in the NIN-like family. This protein is predicted to act as a master regulator of nitrate-promoted seed germination.

SEQ ID NO: 17 encodes a transcription factor gene which contains a sequence-specific DNA binding domain with homology to proteins in the HSF family and is predicted to be involved in the plants response to abiotic stresses such as high temperature, salinity, and drought which adversely affect the survival, growth, and reproduction of plants.

SEQ ID NO: 18 encodes a transcription factor gene which contains a sequence-specific DNA binding domain with homology to proteins in the CO-like family. This CO-like gene is predicted to be involved in the circadian clock and genes controlling meristem identity.

SEQ ID NO: 19 encodes a transcription factor gene which contains a sequence-specific DNA binding domain with homology to proteins in the MIKC family and is predicted to be involved in flower and seed development and may also play a role in the regulation of downstream genes and pathways.

SEQ ID NO: 20 encodes a transcription factor gene which contains a sequence-specific DNA binding domain with homology to proteins in the ARF family. This gene is considered to play a key role in auxin signaling and the molecular mechanisms that control the embryogenic transition of plant somatic cells.

SEQ ID NO: 21 encodes a transcription factor gene which contains a sequence-specific DNA binding domain with homology to proteins in the bHLH family and is predicted to be involved in the regulation of genes involved in biotic and abiotic stress responses.

SEQ ID NO: 22 encodes a transcription factor gene which contains a sequence-specific DNA binding domain with homology to proteins in the LBD family. This gene is strongly associated with abscisic acid (ABA) biosynthesis and may act as a ‘negative regulator’ for growth and developmental processes.

SEQ ID NO: 23 encodes a transcription factor gene which contains a sequence-specific DNA binding domain with homology to proteins in the bHLH family and is predicted to be involved in the regulation of genes involved in biotic and abiotic stress responses.

SEQ ID NO: 24 encodes a transcription factor gene which contains a sequence-specific DNA binding domain belongs to the ERF family and is predicted to be involved in transcriptional regulation of a variety of biological processes related to growth and development, as well as cold and freezing stress tolerance.

It is well known in the art that many plant species, especially polyploid plant species, contain more than one copy of a specific gene and this invention encompasses all copies or homologs of the specific genes identified. It is also routine in the art to use the DNA sequence and protein sequence of the encoded polypeptide of a gene of interest from one crop to carry out homology searches using methods of sequence alignment which are well known in the art to identify the equivalent genes in other crop species.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877. Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. BLASTP protein searches can be performed using default parameters. See, blast.ncbi.nlm.nih.gov/Blast.cgi.

Sequence alignments and percent similarity calculations may be determined using the Megalign program of the LASARGENE bioinformatics computing suite (DNASTAR Inc., Madison, Wis.) or using the AlignX program of the Vector NTI bioinformatics computing suite (Invitrogen, Carlsbad, Calif.). Multiple alignment of the sequences are performed using the Clustal method of alignment (Higgins and Sharp, CABIOS 5:151-153 (1989)) with the default parameters (GAP PENALTY=10, GAP LENGTH PENALTY=10). Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are GAP PENALTY=10, GAP LENGTH PENALTY=10, KTUPLE=2, GAP PENALTY=5, WINDOW=4 and DIAGONALS SAVED=4. A “substantial portion” of an amino acid or nucleotide sequence comprises enough of the amino acid sequence of a polypeptide or the nucleotide sequence of a gene to afford putative identification of that polypeptide or gene, either by manual evaluation of the sequence by one skilled in the art, or by computer-automated sequence comparison and identification using algorithms such as BLAST (Altschul, S. F. et al., J. Mol. Biol. 215:403-410 (1993)) and Gapped Blast (Altschul, S. F. et al., Nucleic Acids Res. 25:3389-3402 (1997)). BLASTN refers to a BLAST program that compares a nucleotide query sequence against a nucleotide sequence database.

Disclosed herein are corn (maize) orthologs of the 24 switchgrass transcription factor genes listed in Table 1 which are useful for practicing this invention. The maize orthologs of the 24 switchgrass transcription factor genes are specified by SEQ ID NOs: 35-56, 110, and 111 (Tables 8 and 10) and reducing their expression alone or in combinations in corn to improve corn performance is included in the scope of this invention. Additional homologs to the maize genes SEQ ID NOs: 35-56, 110, and 111 are provided as SEQ ID NOs: 57-109 and 112-117 (Table 8), and reducing their expression alone or in combination with the 24 maize orthologs of the switchgrass dTFs to improve maize performance is included in the scope of this invention.

Disclosed herein are soybean orthologs of the 24 switchgrass transcription factor genes listed in Table 1 useful for practicing this invention in soybean. The soybean orthologs include genes encoded by SEQ ID NOs: 141-164 (Table 11) and reducing their expression alone or in combinations in soybean to improve soybean performance is included in the scope of this invention. Additional homologs to the soybean genes SEQ ID NOs: 141-164 are provided as SEQ ID NOs: 722-741 (Table 11) and reducing their expression alone or in combination with the 24 soybean orthologs of the switchgrass dTFs to improve soybean performance is included in the scope of this invention.

Disclosed herein are the canola orthologs of the 24 switchgrass transcription factor genes listed in Table 1 useful for practicing this invention in canola. The canola orthologs include genes encoded by SEQ ID NOs: 165-188 (Table 12) and reducing their expression alone or in combinations in canola to improve canola performance is included in the scope of this invention.

Disclosed herein are the rice orthologs of the 24 switchgrass transcription factor genes listed in Table 1 useful for practicing this invention in rice. The rice orthologs include genes encoded by SEQ ID NOs: 189-212 (Table 12) and reducing their expression alone or in combinations in rice to improve rice performance is included in the scope of this invention.

Disclosed herein are the alfalfa orthologs of the 24 switchgrass transcription factor genes listed in Table 1 useful for practicing this invention in alfalfa. The genome sequence for alfalfa (Medicago sativa) is not publically available. The sequence of a close relative to alfalfa, Medicago truncatula was used to find the orthologs to the 24 switchgrass transcription factor genes listed in Table 1. Once the genome sequence of alfalfa (Medicago sativa) is publically available, the alfalfa orthologs can be found by comparison with the Medicago truncatula and switchgrass genes. The Medicago truncatula orthologs include genes encoded by SEQ ID NOs: 213-236 and 762 (Table 13) and reducing the expression of orthologous genes alone or in combinations in alfalfa to improve alfalfa performance is included in the scope of this invention.

Disclosed herein are the sorghum orthologs of the 24 switchgrass transcription factor genes listed in Table 1 useful for practicing this invention in sorghum. The sorghum orthologs include genes encoded by SEQ ID NOs: 237-260 (Table 13) and reducing their expression alone or in combinations in sorghum to improve sorghum performance is included in the scope of this invention.

Disclosed herein are the wheat orthologs of the 24 switchgrass transcription factor genes listed in Table 1 useful for practicing this invention in wheat. The wheat orthologs includes genes encoded by SEQ ID NOs: 261-284 (Table 14) and reducing their expression alone or in combinations in wheat to improve wheat performance is included in the scope of this invention. Two additional homologs to the wheat dTF22 gene (SEQ ID NO: 282) are provided as SEQ ID NOs: 287 and 288 (Table 14), and reducing their expression alone or in combination with the 24 wheat orthologs of the switchgrass dTFs to improve wheat performance is included in the scope of this invention.

Disclosed herein is the Camelina sativa ortholog of dTF22. The Camelina ortholog includes the gene encoded by SEQ ID NO: 285 (Table 15). Identifying the Camelina orthologs of dTF1-dTF21, dTF59, and dTF60 and reducing their expression alone or in combinations in Camelina to improve Camelina performance is included in the scope of this invention.

It will be apparent for anyone skilled in the art to use the genes and the proteins encoded by the genes identified by SEQ ID Nos: 1-24, 35-117, 141-285, 287-288, 544, 545, 722-741, and 762 to identify additional orthologs of the transcription factors from the same crop or equivalent transcription factors genes in any other crop species which will be useful for practicing the invention in a crop-specific manner in any crop.

In an embodiment the expression of one or more of the transcription factor genes listed for each crop species in Tables 1, 5, 6, 8, 10-15 are modified to reduce the expression of the transcription factor in the crop of interest and the performance of the crop is improved.

In an embodiment, the expression of the transcription factor dTF22 encoded by SEQ ID NO: 22 in switchgrass or (SEQ ID NO: 210) in rice, (SEQ ID NO: 56) corn, (SEQ ID NO: 162) in soybean, (SEQ ID NO: 186) in Brassica napus, (SEQ ID NO: 285) in Camelina sativa, (SEQ ID NO: 234) in Medicago truncatula, (SEQ ID NO: 258) in Sorghum bicolor, or (SEQ ID NO: 282) in wheat is reduced in the respective species and the performance of the plant is improved.

In an embodiment, the expression of one or more of the transcription factors encoded by SEQ ID NO: 282, SEQ ID NO: 287, and/or SEQ ID NO: 288 in wheat is reduced and the performance of the plant is improved.

In an embodiment, the expression of one or more of the transcription factors encoded by SEQ ID NO: 162 and/or SEQ ID NO: 741 in soybean is reduced and the performance of the plant is improved.

In an embodiment, the expression of one or more of the transcription factors encoded by SEQ ID NO: 56, SEQ ID NO: 76, SEQ ID NO: 93, and/or SEQ ID NO: 109 in maize is reduced and the performance of the plant is improved.

In an embodiment, the expression of one or more of the transcription factors encoded by SEQ ID NO: 210, SEQ ID NO: 544, and/or SEQ ID NO: 545 in rice is reduced and the performance of the plant is improved.

In an embodiment the expression of two or more of the transcription factor genes listed for each crop species Tables 1, 5, 6, 8, 10-15 are modified to reduce the expression of the transcription factor genes in the crop of interest and the performance of the crop is improved.

In an embodiment the expression of three or more of the transcription factor genes listed in Tables 1, 5, 6, 8, 10-15 are modified to reduce the expression of the transcription factor genes in the crop of interest and the performance of the crop is improved.

In an embodiment the expression of four or more of the transcription factor genes listed for each crop species in Tables 1, 5, 6, 8, 10-15 are modified to reduce the expression of the transcription factor genes in the crop of interest and the performance of the crop is improved.

In an embodiment the expression of five or more of the transcription factor genes listed for each crop species in Tables 1, 5, 6, 8, 10-15 are modified to reduce the expression of the transcription factor genes in the crop of interest and the performance of the crop is improved.

In an embodiment, the expression of the transcription factor is downregulated by Log 2 Fold≤−1 in at least two of the three STR1, STF1 and BMY1 transgenic switchgrass lines presented in Table 1 and at least downregulated in the third transgenic line.

In an embodiment, the expression of the transcription factor is downregulated by Log 2 Fold≤−1 in at least one of the three STR1, STF1 and BMY1 transgenic switchgrass lines presented in Table 1 and at least downregulated in the other two transgenic lines.

Preferred transcription factors of the invention are disclosed based on fold-change in Table 1. Example 1 shows dTF22 is downregulated by all 3 global TFs: STR1, BMY1, and STIF1 and where the down regulation is at least 2-fold change in expression. At least one of the global TFs: STR1, BMY1, and STIF1 downregulates the following dTF3, dTF9, dTF10, dTF14, dTF18 and dTF60. STR1 and BMY downregulate one or more of dTF1, dTF3, dTF7, and dTF22.

In some embodiments, the polynucleotide is downregulated by techniques are said of various new technologies developed and/or used to create new characteristics in plants through genetic variation, the aim being targeted mutagenesis, targeted introduction of new genes or gene silencing (RdDM). Examples of such new breeding techniques are targeted sequence changes facilitated through the use of Zinc finger nuclease (ZFN) technology (ZFN-1, ZFN-2 and ZFN-3, see U.S. Pat. No. 9,145,565, incorporated by reference in its entirety), Oligonucleotide directed mutagenesis (ODM), Cisgenesis and intragenesis, RNA-dependent DNA methylation (RdDM, which does not necessarily change nucleotide sequence but can change the biological activity of the sequence), Grafting (on GM rootstock), Reverse breeding, Agro-infiltration (agro-infiltration “sensu stricto”, agro-inoculation, floral dip), Transcription Activator-Like Effector Nucleases (TALENs, see U.S. Pat. Nos. 8,586,363 and 9,181,535, incorporated by reference in their entireties), the CRISPR/Cas system (see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641), engineered meganuclease re-engineered homing endonucleases, DNA guided genome editing (Gao et al., Nature Biotechnology (2016), doi: 10.1038/nbt.3547, incorporated by reference in its entirety), and synthetic genomics. A complete description of each of these techniques can be found in the report made by the Joint Research Center (JRC) Institute for Prospective Technological Studies of the European Commission in 2011 and titled “New plant breeding techniques—State-of-the-art and prospects for commercial development”.

Modulation of candidate dTF genes are performed through known techniques in the art, such as without limitation, by genetic means, enzymatic techniques, chemicals methods, or combinations thereof. Inactivation may be conducted at the level of DNA, mRNA or protein, and inhibit the expression of one or more candidate dTF genes or the corresponding activity. Preferred inactivation methods affect the expression of the dTF gene and lead to the absence of gene product in the plant cells. It should be noted that the inhibition can be transient or permanent or stable. Inhibition of the protein can be obtained by suppressing or decreasing its activity or by suppressing or decreasing the expression of the corresponding gene. Inhibition can be obtained via mutagenesis of the dTF22 gene. For example, a mutation in the coding sequence can induce, depending upon the nature of the mutation, expression of an inactive protein, or of a reduced-active protein; a mutation at a splicing site can also alter or abolish the protein's function; a mutation in the promoter sequence can induce the absence of expression of said protein, or the decrease of its expression. Mutagenesis can be performed, e.g., by suppressing all or part of the coding sequence or of the promoter, or by inserting an exogenous sequence, e.g., a transposon, into said coding sequence or said promoter. It can also be performed by inducing point mutations, e.g., using ethyl methanesulfonate (EMS) mutagenesis or radiation. The mutated alleles can be detected, e.g., by PCR, by using specific primers of the gene. Rodriguez-Leal et al. describe a promoter editing method that generates a pool of promoter variants that can be screened to evaluate their phenotypic impact (Rodriguez-Leal et al., 2017, Cell, 171, 1-11). This method can be incorporated to downregulate native promoters of each dTF in the crop of interest.

Various high-throughput mutagenesis and splicing methods are described in the prior art. By way of examples, we may cite “TILLING” (Targeting Induced Local Lesions In Genome)-type methods, described by Till, Comai and Henikoff (2007) (R. K. Varshney and R. Tuberosa (eds.), Genomics-Assisted Crop Improvement: Vol. 1: Genomics Approaches and Platforms, 333-349.).

Plants comprising a mutation in the candidate dTF genes that induce inhibition of the protein product are also part of the goal. This mutation can be, e.g., a deletion of all or part of the coding sequence or of the promoter, or it may be a point mutation of said coding sequence or of said promoter.

Advantageously, inhibition of the dTF protein is obtained by silencing or by knock-out techniques on the dTF gene. Various techniques for silencing genes in plants are known. Antisense inhibition or co suppression, described, e.g., in Hamilton and Baulcombe, 1999, Science, vol 286, pp 950-952, is noteworthy. It is also possible to use ribozymes targeting the mRNA of one or more dTF protein. Preferably, silencing of the dTF gene is induced by RNA interference targeting said gene. An interfering RNA (iRNA) is a small RNA that can silence a target gene in a sequence-specific way. Interfering RNA include, specifically, “small interfering RNA” (siRNA) and micro-RNA (miRNA). The most widely-used constructions lead to the synthesis of a pre-miRNA in which the target sequence is present in sense and antisense orientation and separated by a short spacing region. The sense and antisense sequence can hybridize together leading to the formation of a hairpin structure called the pre miRNA. This hairpin structure is maturated leading to the production of the final miRNA. This miRNA will hybridize to the target mRNA which will be cleaved or degraded, as described in Schwab et al (Schwab et al, 2006 The Plant Cell, Vol. 18, 1121-1133) or in Ossowski et al (Ossowski et al, 2008, The plant Journal 53, 674-690).

Inhibition of the dTF proteins can also be obtained by gene editing of the candidate dTF genes. Various methods can be used for gene editing, by using transcription activator-like effector nucleases (TALENs), clustered Regularly Interspaced Short Palindromic Repeats (CRISPR/Cas9) or zinc-finger nucleases (ZFN) techniques (as described in Belhaj et al, 2013, Plant Methods, vol 9, p 39, Chen et al, 2014 Methods Volume 69, Issue 1, p 2-8). Preferably, the inhibition of a dTF protein is obtained by using Clustered Regularly Interspaced Short Palindromic Repeats (CRISPR/Cas9) or CRISPR/Cpf1. The use of this technology in genome editing is well described in the art, for example in Fauser et al. (Fauser et al, 2014, The Plant Journal, Vol 79, p 348-359), and references cited herein. In short, CRISPR is a microbial nuclease system involved in defense against invading phages and plasmids. CRISPR loci in microbial hosts contain a combination of CRISPR-associated (Cas) genes as well as non-coding RNA elements capable of programming the specificity of the CRISPR-mediated nucleic acid cleavage (sgRNA). At least classes (Class I and II) and six types (Types I-VI) of Cas proteins have been identified across a wide range of bacterial hosts. One key feature of each CRISPR locus is the presence of an array of repetitive sequences (direct repeats) interspaced by short stretches of non-repetitive sequences (spacers). The non-coding CRISPR array is transcribed and cleaved within direct repeats into short crRNAs containing individual spacer sequences, which direct Cas nucleases to the target site (protospacer). The Type II CRISPR/Cas is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA: tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Cas9 is thus the hallmark protein of the Type II CRISPR-Cas system, and a large monomeric DNA nuclease guided to a DNA target sequence adjacent to the PAM (protospacer adjacent motif) sequence motif by a complex of two noncoding RNAs: CRISPR RNA (crRNA) and trans-activating crRNA (tracrRNA). The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with an sgRNA can introduce site-specific double strand breaks (DSBs) into genomic DNA of live cells from various organisms. For applications in eukaryotic organisms, codon optimized versions of Cas9, which is originally from the bacterium Streptococcus pyogenes, have been used. The single guide RNA (sgRNA) is the second component of the CRISPR/Cas system that forms a complex with the Cas9 nuclease. sgRNA is a synthetic RNA chimera created by fusing crRNA with tracrRNA. The sgRNA guide sequence located at its 5′ end confers DNA target specificity. Therefore, by modifying the guide sequence, it is possible to create sgRNAs with different target specificities. The canonical length of the guide sequence is 20 bp. In plants, sgRNAs have been expressed using plant RNA polymerase III promoters, such as U6 and U3. Cas9 expression plasmids for use in the methods of the invention can be constructed as described in the art.

The absence of or loss of function in modified engineered plants or plant cells can be verified based on the phenotypic characteristics of their offspring; homozygous plants or plant cells for a mutation inactivating the dTF gene have a content of gene product rate that is lower than that of the wild plants (not carrying the mutation in the gene) from which they originated. Alternatively, a desirable phenotypic characteristic such as biomass yield, seed yield, or seed oil content is measured and is at least 10% higher, preferably at least 20% higher, at least preferably 30% higher, preferably at least 40% higher, preferably at least 50% higher than that of the control plants from which they originated. More preferably, seed yield or seed oil content is at least 60% higher, at least 70% higher, at least 80% higher, at least 90% higher than that of the control plants from which they originated. More preferably, seed yield or seed oil content is at least 100% higher, at least 150% higher, at least 200% higher than that of the control plants from which they originated.

The expression of the target gene or genes in the crops of interest can be reduced by any method known in the art, including the transgene based expression of anti-sense RNA or interfering RNA (RNAi) e.g., siRNA or miRNA or through genome editing to modify the DNA sequence of the genes disclosed herein directly in the plant cell chromosome.

Genome editing is a preferred method for practicing this invention. As used herein the terms “genome editing,” “genome edited,” and “genome modified” are used interchangeably to describe plants with specific DNA sequence changes in their genomes wherein those DNA sequence changes include changes of specific nucleotides, the deletion of specific nucleotide sequences or the insertion of specific nucleotide sequences.

As used herein “method for genome editing” includes all methods for genome editing technologies to precisely remove genes, gene fragments, to insert new DNA sequences into genes, to alter the DNA sequence of control sequences or protein coding regions to reduce or increase the expression of target genes in plant genomes (Belhaj, K. 2013, Plant Methods, 9, 39; Khandagale & Nadal, 2016, Plant Biotechnol Rep, 10, 327). Preferred methods involve the in vivo site-specific cleavage to achieve double stranded breaks in the genomic DNA of the plant genome at a specific DNA sequence using nuclease enzymes and the host plant DNA repair system. There are multiple methods to achieve double stranded breaks in genomic DNA, and thus achieve genome editing, including the use of zinc finger nucleases (ZFN), transcription activator-like effector nucleases (TALENs), engineered meganucleases, and the CRISPR/Cas system (CRISPR is an acronym for clustered, regularly interspaced, short, palindromic repeats and Cas an abbreviation for CRISPR-associated protein) (for review see Khandagal & Nadal, Plant Biotechnol Rep, 2016, 10, 327). US Patent Application 2016/0032297 to Dupont describes these methods in detail. In some cases, the sequence specificity for the target gene in the plant genome is dependent on engineering specific nuclease like zinc finger nucleases (ZFN), which include an engineered DNA-binding zinc finger domain linked to a non-specific endonuclease domain such as FokI, or Tal effector nuclease (TALENS) to recognize the target DNA sequence in the plant genome. The CRISPR/Cas genome editing system is a preferred method because of its sequence targeting flexibility. This technology requires a source of the Cas enzyme and a short single guide RNA (sgRNA, ˜20 bp), DNA, RNA/DNA hybrid or double stranded DNA guide with sequence homology to the target DNA sequence in the plant genome to direct the Cas enzyme to the desired cut site for cleavage and a recognition sequence for binding the Cas enzyme. As used herein the term Cas nuclease includes any nuclease which site-specifically recognizes CRISPR sequences based on guide RNA or DNA sequences and includes Cas9, Cpf1 and others described below. CRISPR/Cas genome editing, is a preferred way to edit the genomes of complex organisms (Sander & Joung, 2013, Nat Biotech, 2014, 32, 347; Wright et al., 2016, Cell, 164, 29) including plants (Zhang et al., 2016, Journal of Genetics and Genomics, 43, 151; Puchta, H., 2016, Plant J., 87, 5; Khandagale & Nadaf, 2016, PLANT BIOTECHNOL REP, 10, 327). US Patent Application 2016/020822 to Dupont has an extensive description of the materials and methods useful for genome editing in plants using the CRISPR Cas9 system and describes many of the uses of the CRISPR/Cas9 system for genome editing of a range of gene targets in crops.

There are many variations of the CRISPR/Cas system that can be used for this technology including the use of wild-type Cas9 from Streptococcus pyogenes (Type II Cas) (Barakate & Stephens, 2016, Frontiers in Plant Science, 7, 765; Bortesi & Fischer, 2015, Biotechnology Advances 5, 33, 41; Cong et al., 2013, Science, 339, 819; Rani et al., 2016, Biotechnology Letters, 1-16; Tsai et al., 2015, Nature biotechnology, 33, 187), the use of a Tru-gRNA/Cas9 in which off-target mutations were significantly decreased (Fu et al., 2014, Nature biotechnology, 32, 279; Osakabe et al., 2016, Scientific Reports, 6, 26685; Smith et al., 2016, Genome biology, 17, 1; Zhang et al., 2016, Scientific Reports, 6, 28566), a high specificity Cas9 (mutated S. pyogenes Cas9) with little to no off target activity (Kleinstiver et al., 2016, Nature 529, 490; Slaymaker et al., 2016, Science, 351, 84), the Type I and Type III Cas Systems in which multiple Cas proteins need to be expressed to achieve editing (Li et al., 2016, Nucleic acids research, 44:e34; Luo et al., 2015, Nucleic acids research, 43, 674), the Type V Cas system using the Cpf1 enzyme (Kim et al., 2016, Nature biotechnology, 34, 863; Toth et al., 2016, Biology Direct, 11, 46; Zetsche et al., 2015, Cell, 163, 759), DNA-guided editing using the NgAgo Argonaute enzyme from Natronobacterium gregoryi that employs guide DNA (Xu et al., 2016, Genome Biology, 17, 186), and the use of a two vector system in which Cas9 and gRNA expression cassettes are carried on separate vectors (Cong et al., 2013, Science, 339, 819). A unique nuclease Cpf1, an alternative to Cas9 has advantages over the Cas9 system in reducing off-target edits which creates unwanted mutations in the host genome. Examples of crop genome editing using the CRISPR/Cpf1 system include rice (Tang et. al., 2017, Nature Plants 3, 1-5; Wu et. al., 2017, Molecular Plant, Mar. 16, 2017) and soybean (Kim et., al., 2017, Nat Commun. 8, 14406).

Methods for constructing the genome modified plant cells and plants include introducing into plant cells a site-specific nuclease to cleave the plant genome at the target site or target sites and the guide sequences. Modification to the DNA sequence at the cleavage site then occur through the plant cells natural DNA repair processes. In a preferred case using the CRISPR system the target site in the plant genome is determined by providing guide RNA sequences.

A “guide polynucleotide” also relates to a polynucleotide sequence that can form a complex with a Cas endonuclease and enables the Cas endonuclease to recognize and optionally cleave a DNA target site. The guide polynucleotide can be a single molecule or a double molecule. The guide polynucleotide sequence can be a RNA sequence, a DNA sequence, or a combination thereof (a RNA-DNA combination sequence).

As used herein “guide RNA” sequences comprise a variable targeting domain, homologous to the target site in the genome and an RNA sequence that interacts with the Cas9 or Cpf1 endonuclease. This variable targeting domain is referred to herein and within the examples as a “guide targeting sequence”. A guide polynucleotide that solely comprises ribonucleic acids is also referred to as a “guide RNA”.

Preferred embodiments include multiplex of gene edits. The method also provides introducing single-guide RNAs (sgRNAs) into plants. The guide RNAs (sgRNAs) include nucleotide sequences that are complementary to the target chromosomal DNA. The sgRNAs can be, for example, engineered single chain guide RNAs that comprise a crRNA sequence (complementary to the target DNA sequence) and a common tracrRNA sequence, or as crRNA-tracrRNA hybrids. The sgRNAs can be introduced into the cell or the organism as a DNA with an appropriate promoter, as an in vitro transcribed RNA, or as a synthesized RNA. Methods for designing the guide RNAs for any target gene of interest are well known in the art as described for example by Brazelton et al. (Brazelton, V. A. et al., 2015, GM Crops & Food, 6, 266-276) and Zhu (Zhu, L. J. 2015, Frontiers in Biology, 10, 289-296).

Target Sequence for Reducing Expression

Examples of mutations that may lead to a reduced activity of the dTF protein are mutations to the coding sequence that give rise to premature stop codons, frame shifts or amino acid changes in the encoded protein. A single guide RNA can be used where the objective is to change a relatively small number of base pairs in the DNA and for example introduce frame-shift mutations resulting in the expression of an inactive or reduced activity protein. Premature stop codons typically lead to the expression of a truncated version of the encoded protein. Depending on the position of the mutation in the coding sequence, a truncated version of a protein may lack one or more domains that are essential to perform its function and/or to interact with substrates or with other proteins, and/or it may lack the ability to fold properly into a functional protein.

In certain preferred embodiments, the guide polynucleotide/Cas endonuclease system can be used to allow for the deletion of a promoter or promoter element of any one the dTF sequences of the invention, wherein the promoter deletion (or promoter element deletion) results in any one of the following or any one combination of the following: a permanently inactivated gene locus, an increased promoter activity (increased promoter strength), an increased promoter tissue specificity, a decreased promoter activity, a decreased promoter tissue specificity, a new promoter activity, an inducible promoter activity, an extended window of gene expression, a modification of the timing or developmental progress of gene expression, a mutation of DNA binding elements and/or an addition of DNA binding elements. Promoter elements to be deleted can be, but are not limited to, promoter core elements, promoter enhancer elements or 35 S enhancer elements (CaMV35S enhancers (Benfey et al, EMBO J, August 1989; 8(8): 2195-2202)). The promoter or promoter fragment to be deleted can be endogenous, artificial, pre-existing, or transgenic to the cell that is being edited.

In another embodiment, the guide polynucleotide/Cas endonuclease system can be used to allow for the deletion of a terminator or terminator element of any one the dTF sequences of the invention, wherein the terminator deletion (or terminator element deletion) results in any one of the following or any one combination of the following: an increased terminator activity (increased terminator strength), an increased terminator tissue specificity, a decreased terminator activity, a decreased terminator tissue specificity, a mutation of DNA binding elements and/or an addition of DNA binding elements. The terminator or terminator fragment to be deleted can be endogenous, artificial, pre-existing, or transgenic to the cell that is being edited.

In yet another embodiment, the genomic sequence of interest to be modified is an intron site of any one the dTF sequences of the invention, wherein the modification consists of inserting an intron enhancing motif into the intron which results in modulation of the transcriptional activity of the gene comprising said intron.

In a further embodiment, methods provide for modifying alternative splicing sites of any one the dTF sequences of the invention resulting in enhanced production of the functional gene transcripts and gene products (proteins).

In additional embodiments, the modification of the dTF sequences of the invention include editing the intron borders of alternatively spliced genes to alter the accumulation of splice variants.

In other embodiments, the guide polynucleotide/Cas endonuclease system can be used to modify or replace a coding sequence of the dTF genome of a plant cell, wherein the modification or replacement results in any one of the following, or any one combination of the following: an increased protein (enzyme) activity, an increased protein functionality, a decreased protein activity, a decreased protein functionality, a site specific mutation, a protein domain swap, a protein knock-out, a new protein functionality, a modified protein functionality.

In some embodiments, the protein knockout is due to the introduction of a stop codon into the coding sequence of interest. In preferred embodiments, the protein knockout is due to the deletion of a start codon into the coding sequence of interest. In yet other embodiments, the guide polynucleotide/Cas endonuclease system can be used with or without a co-delivered polynucleotide sequence to fuse a first coding sequence encoding a nuclear localization signal to a second coding sequence encoding a protein of interest, wherein the protein fusion results in targeting the protein of interest to the nuclease.

The guide RNA/Cas endonuclease system can be used to create frame shift mutations of any one of the dTF sequences of the invention. One or more guide RNAs are used to knockout the dTF genes after the Cas nuclease makes a double strand break and the error prone DNA repair pathway, non-homologous end joining, corrects the break, creating a mutation. The most likely result is a frameshift mutation that would knockout the gene. The targeting strategy involves finding proto-spacers in the exons of the gene that had a PAM sequence, NGG, and was unique in the genome.

The guide RNA/Cas endonuclease system can be used to allow for the deletion of a promoter element from any one of the dTF sequences of the invention. Promoter elements, such as enhancer elements, are often introduced in promoters driving gene expression cassettes in multiple copies for trait gene testing or to produce transgenic plants expressing specific trait. Enhancer elements can be, but are not limited to, a 35S enhancer element (Benfey et al, EMBO J, August 1989; 8(8): 2195-2202). In some plants (events), the enhancer elements can cause an unwanted phenotype, a yield drag, or a change in expression pattern of the trait of interest that is not desired. It may be desired to remove the extra copies of the enhancer element while keeping the trait gene cassettes intact at their integrated genomic location. The guide RNA/Cas endonuclease can be used to remove the unwanted enhancing element from the plant genome. A guide RNA can be designed to contain a variable targeting region, or “guide target sequence” targeting a sequence of 12-30 bps adjacent to a NGG (PAM) in the enhancer. The Cas endonuclease can make cleavage to remove one or multiple enhancers. The guide RNA/Cas endonuclease system can be introduced by either Agrobacterium or particle gun bombardment. Alternatively, two different guide RNAs (targeting two different genomic target sites) can be used to remove multiple enhancer elements from the genome of a plant.

In some embodiments, the genome modified plant has improved performance as compared to a plant of the same type which does not have the genome modification. The improved performance of the genome modified plant includes for example, higher photosynthesis rates, reduced photorespiration rates, higher biomass yield, higher seed yield, improved harvest index, higher oil content, improved nutritional composition, drought resistance, flood resistance, disease resistance, faster seed germination and plant emergence, and/or improved seedling vigor. The genome modified plant can have a CO₂ assimilation rate that is higher than for a corresponding control plant not comprising the genome modification. For example, the genome modified plant can have a CO₂ assimilation rate that is at least 5% higher, at least 10% higher, at least 20% higher, at least 40% higher, at least 60% higher, at least 100% higher, at least 200% higher or at least 400% higher than for a corresponding control plant not comprising the genome modification.

The genome modified plant can also have a transpiration rate that is lower than for a corresponding control plant not comprising the genome modification. For example, the genome modified plant can have a transpiration rate that is at least 5% lower, at least 10% lower, at least 20% lower, at least 40% lower, at least 60% lower or at least 100% lower than for a corresponding control plant not comprising the genome modification.

The genome modified plant can have a seed yield or a seed oil content that is higher than for a corresponding control plant not comprising the genome modification. For example, the genome modified plant can have a seed yield or seed oil content that is at least 5% higher, at least 10% higher, at least 20% higher, at least 40% higher, at least 60% higher, at least 80% higher or at least 100% higher, than for a corresponding control plant not comprising the genome modification.

The genome modified plant can have a seed yield that is higher than for a corresponding control plant not comprising the genome modification. For example, the genome modified plant can have a seed yield that is at least 5% higher, at least 10% higher, at least 20% higher, at least 40% higher, at least 60% higher, at least 80% higher or at least 100% higher, than for a corresponding control plant not comprising the genome modification.

Plants of Interest

Plants encompass all annual and perennial monocotyledonous or dicotyledonous plants. Preferred dicotyledonous plants are selected in particular from the dicotyledonous crop plants such as sunflower, lettuce, the genus Brassica, very particularly the species napus (oilseed rape), campestris (beet), oleracea cv Tastie (cabbage), oleracea cv Snowball Y (cauliflower) and oleracea cv Emperor (broccoli), cabbage, melon, pumpkin/squash or zucchini and others; soybean, alfalfa, pea, beans, peanut, tomato, potato, sweet potato, yams carrot, flax, cotton, hemp, cucumber, spinach, carrot, sugar beet and the various tree, nut and grapevine species, in particular banana and kiwi fruit. Preferred monocotyledonous plants include maize, rice, wheat, sugarcane, sorghum, oats and barley.

Of interest are oilseed plants including Camelina (false flax); Brassica species such as B. campestris, B. napus, B. rapa, B. carinata (mustard, oilseed rape or turnip rape); Cannabis sativa (hemp); Carthamus tinctorius (safflower); Cocos nucifera (coconut); Crambe abyssinica (crambe); Elaeis guinensis (African oil palm); Elaeis oleifera (American oil palm); Glycine max (soybean); Gossypium hirsutum (American cotton); Gossypium barbadense (Egyptian cotton); Gossypium herbaceum (Asian cotton); Helianthus annuus (sunflower); Jatropha curcas (jatropha); Linum usitatissimum (linseed or flax); Oenothera biennis (evening primrose); Olea europaea (olive); Oryza sativa (rice); Ricinus communis (castor); Sesamum indicum (sesame); Thlaspi caerulescens (pennycress); Triticum species (wheat); Zea mays (maize or corn), and various nut species such as, for example, walnut or almond.

Plants selected from the group, corn (maize), sugarcane, sorghum, millet, cassava, soybean, canola, cotton, wheat, rice, potato, tomato, pulses, vegetables, sunflower, safflower and Camelina are examples of particularly useful plants for performance improvement using the methods, target genes for altered expression to achieve improved plant performance and genome inserts to alter the expression of the target gene(s) are disclosed herein.

Transcription factor genes, including crop-specific transcription factor gene sequences in preferred crop species useful as targets for down regulation, alone or in combinations, to improve crop performance are described herein. Methods of downregulating these genes in these crops including site-specific nucleases, guide RNAs, guide RNA-DNA hybrids and guide DNAs, DNA constructs useful in the methods are described herein. Methods for introducing the site-specific nuclease and guide RNAs into plant cells and plant tissues are also described herein and methods for identifying plant cells, plant tissue and fertile plants having reduced expression of the transcription factor genes made using these methods are disclosed herein. As used herein, “transgenic” refers to an organism in which a nucleic acid fragment containing a heterologous or non-native” nucleotide sequence has been introduced. The genome inserts introduced into the plants are stable, inheritable and impart improved plant performance.

Modified Plant Genomes Using CRISPR/Cas, Guide RNAs

Examples of simultaneous CRISPR/Cas9 or CRISPR/Cpf1 gene editing at multiple target sites, or multiplex genome editing, have been described for both mammalian cells and plants, and can be achieved by expressing one or more sgRNAs to target multiple genome sites within the organism. This has been demonstrated in rice with the use of seven sgRNAs for editing (Ma et al., 2015, Mol Plant, 8, 1274). It is therefore an objective of this invention to use multiple sgRNAs to direct the insertion of a specific DNA sequence to multiple sites in the plant genome using one or more of the previous embodiments of the invention. Example 3 provides inactivation of dTF22 expression.

Methods for DNA Modification at the Target Site

The methods for achieving the genome modification are described using the CRISPR/Cas9 system although it will be appreciated that other variations of the CRISPR/Cas9 system can also be used including one that uses guide DNA sequences. The method requires the introduction of the site-specific nuclease and guide RNA into the nucleus of plant cells from the target crop. These may vary for different crop species or due to preference or skill set of the crop scientists.

One skilled in the art can produce and introduce proteins or DNA into many crop types using plant cell protoplasts. Preferably the plant protoplasts once genome edited can be regenerated into stable fertile plants suitable for crop breeding programs. For example, protoplast transformation and hence genome editing is useful for modifying the genomes of Camelina, as disclosed herein but also for canola, soybean, corn, rice, wheat, potato, alfalfa, tomato, cotton, barley and many other crops of interest. The Cas9 nuclease enzyme can be combined with the gRNAs and protein/RNA particles which can then be introduced into the plant protoplasts.

Methods for Identifying or Selecting Plant Cells with the Targeted Genome Edits

Methods of Plant Transformation

Known transformations methods can be used downregulate one or more gene sequences of the invention.

Vectors

Several plant transformation vector options are available, including those described in Gene Transfer to Plants, 1995, Potrykus et al., eds., Springer-Verlag Berlin Heidelberg New York, Transgenic Plants: A Production System for Industrial and Pharmaceutical Proteins, 1996, Owen et al., eds., John Wiley & Sons Ltd. Eng, and Methods in Plant Molecular Biology: A Laboratory Course Manual, 1995, Maliga et al., eds., Cold Spring Laboratory Press, New York. Plant transformation vectors generally include one or more coding sequences of interest under the transcriptional control of 5′ and 3′ regulatory sequences, including a promoter, a transcription termination and/or polyadenylation signal, and a selectable or screenable marker gene.

Many vectors are available for transformation using Agrobacterium tumefaciens. These typically carry at least one T-DNA sequence and include vectors such as pBIN19. Typical vectors suitable for Agrobacterium transformation include the binary vectors pCIB200 and pCIB2001, as well as the binary vector pCIB 10 and hygromycin selection derivatives thereof (See, for example, U.S. Pat. No. 5,639,949).

Transformation without the use of Agrobacterium tumefaciens circumvents the requirement for T-DNA sequences in the chosen transformation vector and consequently vectors lacking these sequences are utilized in addition to vectors such as the ones described above which contain T-DNA sequences. The choice of vector for transformation techniques that do not rely on Agrobacterium depends largely on the preferred selection for the species being transformed. Typical vectors suitable for non-Agrobacterium transformation include pCIB3064, pSOG 19, and pSOG35. (See, for example, U.S. Pat. No. 5,639,949). Alternatively, DNA fragments containing the transgene and the necessary regulatory elements for expression of the transgene can be excised from a plasmid and delivered to the plant cell using microprojectile bombardment-mediated methods.

Protocols

Transformation protocols as well as protocols for introducing nucleotide sequences into plants may vary depending on the type of plant or plant cell targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606), Agrobacterium-mediated transformation (Townsend et al., U.S. Pat. No. 5,563,055; Zhao et al. WO US98/01268), direct gene transfer (Paszkowski et al. (1984) EMBO J. 3:2717-2722), and ballistic particle acceleration (see, for example, Sanford et al., U.S. Pat. No. 4,945,050; Tomes et al. (1995) Plant Cell, Tissue, and Organ Culture: Fundamental Methods, ed. Gamborg and Phillips (Springer-Verlag, Berlin); and McCabe et al. Biotechnology 6:923-926 (1988)). Also see Weissinger et al. Ann. Rev. Genet. 22:421-477 (1988); Sanford et al. Particulate Science and Technology 5:27-37 (1987) (onion); Christou et al. Plant Physiol. 87:671-674 (1988) (soybean); McCabe et al. (1988) BioTechnology 6:923-926 (soybean); Finer and McMullen In Vitro Cell Dev. Biol. 27P:175-182 (1991) (soybean); Singh et al. Theor. Appl. Genet. 96:319-324 (1998)(soybean); Dafta et al. (1990) Biotechnology 8:736-740 (rice); Klein et al. Proc. Natl. Acad. Sci. USA 85:4305-4309 (1988) (maize); Klein et al. Biotechnology 6:559-563 (1988) (maize); Tomes, U.S. Pat. No. 5,240,855; Buising et al., U.S. Pat. Nos. 5,322,783 and 5,324,646; Tomes et al. (1995) in Plant Cell, Tissue, and Organ Culture: Fundamental Methods, ed. Gamborg (Springer-Verlag, Berlin) (maize); Klein et al. Plant Physiol. 91:440-444 (1988) (maize); Fromm et al. Biotechnology 8:833-839 (1990) (maize); Hooykaas-Van Slogteren et al. Nature 311:763-764 (1984); Bowen et al., U.S. Pat. No. 5,736,369 (cereals); Bytebier et al. Proc. Natl. Acad. Sci. USA 84:5345-5349 (1987) (Liliaceae); De Wet et al. in The Experimental Manipulation of Ovule Tissues, ed. Chapman et al. (Longman, N.Y.), pp. 197-209 (1985) (pollen); Kaeppler et al. Plant Cell Reports 9:415-418 (1990) and Kaeppler et al. Theor. Appl. Genet. 84:560-566 (1992) (whisker-mediated transformation); D'Halluin et al. Plant Cell 4:1495-1505 (1992) (electroporation); Li et al. Plant Cell Reports 12:250-255 (1993) and Christou and Ford Annals of Botany 75:407-413 (1995) (rice); Osjoda et al. Nature Biotechnology 14:745-750 (1996) (maize via Agrobacterium tumefaciens). References for protoplast transformation and/or gene gun for Agrisoma technology are described in WO 2010/037209. Methods for transforming plant protoplasts are available including transformation using polyethylene glycol (PEG), electroporation, and calcium phosphate precipitation (see for example Potrykus et al., 1985, Mol. Gen. Genet., 199, 183-188; Potrykus et al., 1985, Plant Molecular Biology Reporter, 3, 117-128). Methods for plant regeneration from protoplasts have also been described [Evans et al., in Handbook of Plant Cell Culture, Vol 1, (Macmillan Publishing Co., New York, 1983); Vasil, I K in Cell Culture and Somatic Cell Genetics (Academic, Oro, 1984)].

Transformation protocols as well as protocols for introducing nucleotide sequences into plants may vary depending on the type of plant or plant cell, i.e., monocot or dicot, targeted for transformation.

Suitable methods of introducing nucleotide sequences into plant cells and subsequent insertion into the plant genome are described in US 2010/0229256 A1 to Somleva & Ali and US 2012/0060413 to Somleva et al.

The transformed cells are grown into plants in accordance with conventional techniques. See, for example, McCormick et al., 1986, Plant Cell Rep. 5: 81-84. These plants may then be grown, and either pollinated with the same transformed variety or different varieties, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

Procedures for in planta transformation can be simple. Tissue culture manipulations and possible somaclonal variations are avoided and only a short time is required to obtain transgenic plants. However, the frequency of transformants in the progeny of such inoculated plants is relatively low and variable. At present, there are very few species that can be routinely transformed in the absence of a tissue culture-based regeneration system. Stable Arabidopsis transformants can be obtained by several in planta methods including vacuum infiltration (Clough & Bent, 1998, The Plant J. 16: 735-743), transformation of germinating seeds (Feldmann & Marks, 1987, Mol. Gen. Genet. 208: 1-9), floral dip (Clough and Bent, 1998, Plant J. 16: 735-743), and floral spray (Chung et al., 2000, Transgenic Res. 9: 471-476). Other plants that have successfully been transformed by in planta methods include rapeseed and radish (vacuum infiltration, Ian and Hong, 2001, Transgenic Res., 10: 363-371; Desfeux et al., 2000, Plant Physiol. 123: 895-904), Medicago truncatula (vacuum infiltration, Trieu et al., 2000, Plant J. 22: 531-541), camelina (floral dip, WO/2009/117555 to Nguyen et al.), and wheat (floral dip, Zale et al., 2009, Plant Cell Rep. 28: 903-913). In planta methods have also been used for transformation of germ cells in maize (pollen, Wang et al. 2001, Acta Botanica Sin., 43, 275-279; Zhang et al., 2005, Euphytica, 144, 11-22; pistils, Chumakov et al. 2006, Russian J. Genetics, 42, 893-897; Mamontova et al. 2010, Russian J. Genetics, 46, 501-504) and Sorghum (pollen, Wang et al. 2007, Biotechnol. Appl. Biochem., 48, 79-83).

Selection

Following transformation by any one of the methods described above, the following procedures can be used to obtain a transformed plant expressing the transgenes: select the plant cells that have been transformed on a selective medium; regenerate the plant cells that have been transformed to produce differentiated plants; select transformed plants expressing the DNA construct for introducing the targeted insertion of the DNA sequence elements producing the desired level of desired polypeptide(s) in the desired tissue and cellular location.

The cells that have been transformed may be grown into plants in accordance with conventional techniques. See, for example, McCormick et al. Plant Cell Reports 5:81-84 (1986). These plants may then be grown, and either pollinated with the same transformed variety or different varieties, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

Transgenic plants can be produced using conventional techniques to express any genes of interest in plants or plant cells (Methods in Molecular Biology, 2005, vol. 286, Transgenic Plants: Methods and Protocols, Pena L., ed., Humana Press, Inc. Totowa, N.J.; Shyamkumar Barampuram and Zhanyuan J. Zhang, Recent Advances in Plant Transformation, in James A. Birchler (ed.), Plant Chromosome Engineering: Methods and Protocols, Methods in Molecular Biology, vol. 701, Springer Science+Business Media). Typically, gene transfer, or transformation, is carried out using explants capable of regeneration to produce complete, fertile plants. Generally, a DNA or an RNA molecule to be introduced into the organism is part of a transformation vector. A large number of such vector systems known in the art may be used, such as plasmids. The components of the expression system can be modified, e.g., to increase expression of the introduced nucleic acids. For example, truncated sequences, nucleotide substitutions or other modifications may be employed. Expression systems known in the art may be used to transform virtually any plant cell under suitable conditions. A transgene comprising a DNA molecule encoding a gene of interest is preferably stably transformed and integrated into the genome of the host cells. Transformed cells are preferably regenerated into whole fertile plants. Detailed description of transformation techniques are within the knowledge of those skilled in the art.

Plant promoters can be selected to control the expression of the transgene in different plant tissues or organelles for all of which methods are known to those skilled in the art (Gasser & Fraley, 1989, Science 244: 1293-1299). In one embodiment, promoters are selected from those of eukaryotic or synthetic origin that are known to yield high levels of expression in plants and algae. In a preferred embodiment, promoters are selected from those that are known to provide high levels of expression in monocots.

Constitutive promoters include, for example, the core promoter of the Rsyn7 promoter and other constitutive promoters disclosed in WO 99/43838 and U.S. Pat. No. 6,072,050, the core CaMV 35S promoter (Odell et al., 1985, Nature 313: 810-812), rice actin (McElroy et al., 1990, Plant Cell 2: 163-171), ubiquitin (Christensen et al., 1989, Plant Mol. Biol. 12: 619-632; Christensen et al., 1992, Plant Mol. Biol. 18: 675-689), pEMU (Last et al., 1991, Theor. Appl. Genet. 81: 581-588), MAS (Velten et al., 1984, EMBO J. 3: 2723-2730), and ALS promoter (U.S. Pat. No. 5,659,026). Other constitutive promoters are described in U.S. Pat. Nos. 5,608,149; 5,608,144; 5,604,121; 5,569,597; 5,466,785; 5,399,680; 5,268,463; and 5,608,142.

“Tissue-preferred” promoters can be used to target gene expression within a particular tissue. Compared to chemically inducible systems, developmentally and spatially regulated stimuli are less dependent on penetration of external factors into plant cells. Tissue-preferred promoters include those described by Van Ex et al., 2009, Plant Cell Rep. 28: 1509-1520; Yamamoto et al., 1997, Plant J. 12: 255-265; Kawamata et al., 1997, Plant Cell Physiol. 38: 792-803; Hansen et al., 1997, Mol. Gen. Genet. 254: 337-343; Russell et al., 199), Transgenic Res. 6: 157-168; Rinehart et al., 1996, Plant Physiol. 112: 1331-1341; Van Camp et al., 1996, Plant Physiol. 112: 525-535; Canevascini et al., 1996, Plant Physiol. 112: 513-524; Yamamoto et al., 1994, Plant Cell Physiol. 35: 773-778; Lam, 1994, Results Probl. Cell Differ. 20: 181-196, Orozco et al., 1993, Plant Mol. Biol. 23: 1129-1138; Matsuoka et al., 1993, Proc. Natl. Acad. Sci. USA 90: 9586-9590, and Guevara-Garcia et al., 1993, Plant J. 4: 495-505. Such promoters can be modified, if necessary, for weak expression.

Any of the described promoters can be used to control the expression of one or more of the genes of the invention, their homologs and/or orthologs as well as any other genes of interest in a defined spatiotemporal manner.

Expression Cassettes

Nucleic acid sequences intended for expression in transgenic plants are first assembled in expression cassettes behind a suitable promoter active in plants. The expression cassettes may also include any further sequences required or selected for the expression of the transgene. Such sequences include, but are not restricted to, transcription terminators, extraneous sequences to enhance expression such as introns, vital sequences, and sequences intended for the targeting of the gene product to specific organelles and cell compartments. These expression cassettes can then be transferred to the plant transformation vectors described infra.

A variety of transcriptional terminators are available for use in expression cassettes. These are responsible for the termination of transcription beyond the transgene and the correct polyadenylation of the transcripts. Appropriate transcriptional terminators are those that are known to function in plants and include the CaMV 35S terminator, the tml terminator, the nopaline synthase terminator and the pea rbcS E9 terminator. These are used in both monocotyledonous and dicotyledonous plants.

Individual plants within a population of transgenic plants that express a recombinant gene(s) may have different levels of gene expression. The variable gene expression is due to multiple factors including multiple copies of the recombinant gene, chromatin effects, and gene suppression. Accordingly, a phenotype of the transgenic plant may be measured as a percentage of individual plants within a population. The yield of a plant can be measured simply by weighing. The yield of seed from a plant can also be determined by weighing. The increase in seed weight from a plant can be due to a number of factors, an increase in the number or size of the seed pods, an increase in the number of seed or an increase in the number of seed per plant. In the laboratory or greenhouse seed yield is usually reported as the weight of seed produced per plant and in a commercial crop production setting yield is usually expressed as weight per acre or weight per hectare.

A recombinant DNA construct including a plant-expressible gene or other DNA of interest is inserted into the genome of a plant by a suitable method. Suitable methods include, for example, Agrobacterium tumefaciens-mediated DNA transfer, direct DNA transfer, liposome-mediated DNA transfer, electroporation, co-cultivation, diffusion, particle bombardment, microinjection, gene gun, calcium phosphate coprecipitation, viral vectors, and other techniques. Suitable plant transformation vectors include those derived from a Ti plasmid of Agrobacterium tumefaciens. In addition to plant transformation vectors derived from the Ti or root-inducing (Ri) plasmids of Agrobacterium, alternative methods can be used to insert DNA constructs into plant cells. A transgenic plant can be produced by selection of transformed seeds or by selection of transformed plant cells and subsequent regeneration.

In one embodiment, the transgenic plants are grown (e.g., on soil) and harvested. In one embodiment, above ground tissue is harvested separately from below ground tissue. Suitable above ground tissues include shoots, stems, leaves, flowers, grain, and seed. Exemplary below ground tissues include roots and root hairs. In one embodiment, whole plants are harvested and the above ground tissue is subsequently separated from the below ground tissue.

Genetic constructs may encode a selectable marker to enable selection of transformation events. There are many methods that have been described for the selection of transformed plants [for review see (Miki et al., Journal of Biotechnology, 2004, 107, 193-232) and references incorporated within]. Selectable marker genes that have been used extensively in plants include the neomycin phosphotransferase gene nptII (U.S. Pat. Nos. 5,034,322, 5,530,196), hygromycin resistance gene (U.S. Pat. No. 5,668,298, Waldron et al., (1985), Plant Mol Biol, 5:103-108; Zhijian et al., (1995), Plant Sci, 108:219-227), the bar gene encoding resistance to phosphinothricin (U.S. Pat. No. 5,276,268), the expression of aminoglycoside 3″-adenyltransferase (aadA) to confer spectinomycin resistance (U.S. Pat. No. 5,073,675), the use of inhibition resistant 5-enolpyruvyl-3-phosphoshikimate synthetase (U.S. Pat. No. 4,535,060) and methods for producing glyphosate tolerant plants (U.S. Pat. Nos. 5,463,175; 7,045,684). Other suitable selectable markers include, but are not limited to, genes encoding resistance to chloramphenicol (Herrera Estrella et al., (1983), EMBO J, 2:987-992), methotrexate (Herrera Estrella et al., (1983), Nature, 303:209-213; Meijer et al, (1991), Plant Mol Biol, 16:807-820); streptomycin (Jones et al., (1987), Mol Gen Genet, 210:86-91); bleomycin (Hille et al., (1990), Plant Mol Biol, 7:171-176); sulfonamide (Guerineau et al., (1990), Plant Mol Biol, 15:127-136); bromoxynil (Stalker et al., (1988), Science, 242:419-423); glyphosate (Shaw et al., (1986), Science, 233:478-481); phosphinothricin (DeBlock et al., (1987), EMBO J, 6:2513-2518).

Methods of plant selection that do not use antibiotics or herbicides as a selective agent have been previously described and include expression of glucosamine-6-phosphate deaminase to inactive glucosamine in plant selection medium (U.S. Pat. No. 6,444,878) and a positive/negative system that utilizes D-amino acids (Erikson et al., Nat Biotechnol, 2004, 22, 455-8). European Patent Publication No. EP 0 530 129 A1 describes a positive selection system which enables the transformed plants to outgrow the non-transformed lines by expressing a transgene encoding an enzyme that activates an inactive compound added to the growth media. U.S. Pat. No. 5,767,378 describes the use of mannose or xylose for the positive selection of transgenic plants.

Methods for positive selection using sorbitol dehydrogenase to convert sorbitol to fructose for plant growth have also been described (WO 2010/102293). Screenable marker genes include the beta-glucuronidase gene (Jefferson et al., 1987, EMBO J. 6: 3901-3907; U.S. Pat. No. 5,268,463) and native or modified green fluorescent protein gene (Cubitt et al., 1995, Trends Biochem. Sci. 20: 448-455; Pan et al., 1996, Plant Physiol. 112: 893-900).

Transformation events can also be selected through visualization of fluorescent proteins such as the fluorescent proteins from the nonbioluminescent Anthozoa species which include DsRed, a red fluorescent protein from the Discosoma genus of coral (Matz et al. (1999), Nat Biotechnol 17: 969-73). An improved version of the DsRed protein has been developed (Bevis and Glick (2002), Nat Biotech 20: 83-87) for reducing aggregation of the protein.

Visual selection can also be performed with the yellow fluorescent proteins (YFP) including the variant with accelerated maturation of the signal (Nagai, T. et al. (2002), Nat Biotech 20: 87-90), the blue fluorescent protein, the cyan fluorescent protein, and the green fluorescent protein (Sheen et al. (1995), Plant J 8: 777-84; Davis and Vierstra (1998), Plant Molecular Biology 36: 521-528). A summary of fluorescent proteins can be found in Tzfira et al. (Tzfira et al. (2005), Plant Molecular Biology 57: 503-516) and Verkhusha and Lukyanov (Verkhusha, V. V. and K. A. Lukyanov (2004), Nat Biotech 22: 289-296) whose references are incorporated in entirety. Improved versions of many of the fluorescent proteins have been made for various applications. It will be apparent to those skilled in the art how to use the improved versions of these proteins or combinations of these proteins for selection of transformants.

The plants modified for enhanced performance by reducing the expression of the transcription factor genes or transcription factor gene combinations may be combined or stacked with input traits by crossing or plant breeding. Useful input traits include herbicide resistance and insect tolerance, for example a plant that is tolerant to the herbicide glyphosate and that produces the Bacillus thuringiensis (BT) toxin. Glyphosate is a herbicide that prevents the production of aromatic amino acids in plants by inhibiting the enzyme 5-enolpyruvylshikimate-3-phosphate synthase (EPSP synthase). The overexpression of EPSP synthase in a crop of interest allows the application of glyphosate as a weed killer without killing the modified plant (Suh, et al., J. M Plant Mol. Biol. 1993, 22, 195-205). BT toxin is a protein that is lethal to many insects providing the plant that produces it protection against pests (Barton, et al. Plant Physiol. 1987, 85, 1103-1109). Other useful herbicide tolerance traits include but are not limited to tolerance to Dicamba by expression of the dicamba monoxygenase gene (Behrens et al, 2007, Science, 316, 1185), tolerance to 2,4-D and 2,4-D choline by expression of a bacterial aad-1 gene that encodes for an aryloxyalkanoate dioxygenase enzyme (Wright et al., Proceedings of the National Academy of Sciences, 2010, 107, 20240), glufosinate tolerance by expression of the bialophos resistance gene (bar) or the pat gene encoding the enzyme phosphinotricin acetyl transferase (Droge et al., Planta, 1992, 187, 142), as well as genes encoding a modified 4-hydroxyphenylpyruvate dioxygenase (HPPD) that provides tolerance to the herbicides mesotrione, isoxaflutole, and tembotrione (Siehl et al., Plant Physiol, 2014, 166, 1162). The plants modified for enhanced yield by reducing the expression of the transcription factor genes or transcription factor gene combinations may be combined or stacked with other genes which improve plant performance.

Unless otherwise defined, all technical and scientific terms used herein have the same meaning as commonly understood by one of ordinary skill in the art to which this present invention pertains. Exemplary methods and materials are described below, although methods and materials similar or equivalent to those described herein can also be used in the practice of the present invention and will be apparent to those of skill in the art.

All patents, publications and other references mentioned herein are incorporated by reference in their entirety. In case of conflict, the present specification, including definitions, will control. The materials, methods, and examples are illustrative only and not intended to be limiting.

### EXAMPLES

### Example 1. Identification of Downregulated Transcription Factors in Switchgrass Lines Expressing Global Transcription Factors

Transgenic overexpression of global transcription factors STR1, STIF1, and BMY1 (US 2016/0194650; WO2014100289) was previously shown to increase yield in switchgrass. Although the use of the global transcription factors is useful for the production of biomass, for most crops it is more important to increase the yield of the harvested product which is seed. It is also important to be able to identify the downstream genes responsible for the overall impact of the global transcription factors to be able to develop plants with traits useful for the particular crop of interest without unwanted outcomes. These genetically engineered switchgrass lines are invaluable sources of new information for identifying other transcription factors genes whose altered expression or activity was important for this yield increase. Global gene expression profiling using an Affymetrix switchgrass cDNA GeneChip was performed as described below to determine the changes in gene expression in the high yielding lines for all of the 40,000 known genes in the switchgrass genome.

Global Gene Expression Analysis and Data Mining.

Three pooled RNA samples, each from three independent transgenic switchgrass plants confirmed to express STR1, STIF1 and BMY1, were used as biological replicates for the microarray gene expression analysis. After total RNA QC analysis, hybridization and scanning to the Affymetrix switchgrass GeneChip containing probes was performed to query approximately 43,344 transcripts (Zhang et al., 2013, Plant Journal 74: 160-173) using the manufacturer's instructions (http://www.affymetrix.com). Raw numeric values representing the signal of each feature were imported into AffylmGUI and the data were background corrected, normalized, and summarized using Robust Multiarray Averaging (RMA). A linear model was then used to average data between replicate arrays and to detect differential expression. The quality of gene data was assessed using box and scatter plots. The box plot was used to compare the intensity distributions of all samples. The distributions of log 2 ratios among the samples were similar. The scatter plot was used to assess gene expression variation between the replicates. Data from STR1, STIF1, and BMY1 lines were compared to wild-type lines and genes with significant probe sets (FDR<0.1) with ≥2.0-fold changes were considered as differentially expressed.

Identification and Functional Annotations of “Differentially Expressed Genes” Regulated by the TFs.

Since the whole genome sequence of switchgrass is not well annotated, reciprocal BLAST analysis (switchgrass-rice-maize-sorghum) was used to obtain the functional annotations and their corresponding orthologs for the differentially expressed genes. Reciprocal BLAST is a common computational method for predicting ‘putative orthologues’. The BLAST algorithm calculates percent sequence identity and performs a statistical analysis of the similarity between the two sequences. Typically, this uses a first BLAST that involves BLASTing a query sequence, for example a differentially expressed switchgrass transcript, against a database of gene sequences from an organism of interest, such as rice, maize, or sorghum. The database of gene sequences can be a publicly available database, such as the databases available at the National Center for Biotechnology Information (NCBI) or a completely sequenced genome. BLASTN or TBLASTX are generally used when starting from a nucleotide sequence, and BLASTP or TBLASTN when starting from a protein sequence. The BLAST results may optionally be filtered. The full-length sequences of either the filtered results or non-filtered results are then BLASTed back (second BLAST) against sequences from the organism from which the query sequence is derived, in our case switchgrass. The results of the first and second BLASTs are then compared. If this returns the switchgrass gene originally used as the highest scorer, then the two genes are considered putative orthologues.

Using the Plants TF database v 3.0, 77 genes that are predicted to be transcription factors with a DNA-binding domain were identified. These downstream transcription factors (dTFs) belonged to diverse family of transcription factors such as MYB, bZIP, ERF, bHLH, NAC, C2H2, G2-like, CO-like, WRKY, and HD-Zip. Interestingly, we found that the genes encoding proteins with MYB and bZIP class were the most enriched genes regulated in STR1 and BMY1, and several of these orthologous genes are predicted to be involved with agronomic traits such as biomass yield, grain yield, and abiotic stress tolerance in model and other crop plants.

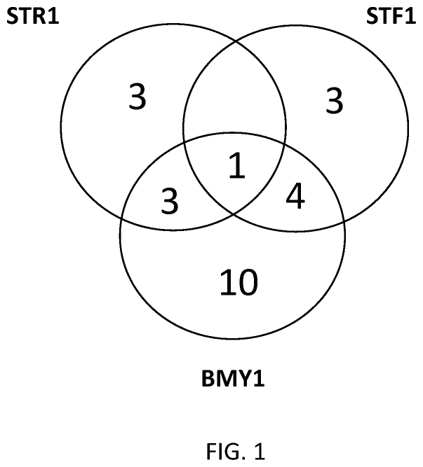

For the purpose of this experiment we focused on the dTFs that are downregulated since they can be inactivated or their expression can be reduced alone or in combinations to increase crop performance using a range of methods well known in the art including genome editing. Genome editing has the advantage that it may enable the development of performance enhancing traits for major food crops with lower government regulatory hurdles. The individual dTFs that were downregulated by more than two-fold (Log 2 Fold ≤−1) by STR1, STFI, or BMY1 are shown in Table 1. 16 dTFs were downregulated by only one of the global transcription factors. Using the two-fold cut off (Log 2 ≤−1), eight dTFs were downregulated by two or more global regulatory genes selected from STR1, STIF1, and BMY1 (FIG. 1). Using the two-fold cut off only one gene, dTF22, was downregulated by all three global transcription factors STR1, STIF1, and BMY1. However there were several dTFs that were down regulated by all three global transcription factors STR1, STIF1, and BMY1 that didn't meet the 2 fold cutoff, including dTF3, dTF9, dTF10, dTF14, dTF17, dTF18, and dTF60.

TABLE 1
Candidate dTF genes that are down-regulated by one or more global transcription factors

| | | | | STR1 Log2 | STIF1 Log2 | BMY1 Log2 |
|---|---|---|---|---|---|---|
| | | | Gene | Fold Change | Fold Change | Fold Change |
| Gene | Switchgrass Gene ID¹ | Gene annotation | Family | (Down regulated)² | (Down regulated) | (Down regulated) |
| dTF1 | Pavirv00029177m | MYB family | MYB | −1.59 | 0.37 | −2.11 |
| | (Gene: SEQ ID NO: 1, | transcription | | (Down) | | (Down) |
| | Protein: SEQ ID NO: 289) | factor, putative, | | | | |
| | | expressed | | | | |
| dTF2 | Pavirv00003507m | homeodomain- | MYB | 0.12 | −1.09 | −1.83 |
| | (Gene: SEQ ID NO: 2, | related, putative, | | | (Down) | (Down) |
| | Protein: SEQ ID NO: 290) | expressed | | | | |
| dTF3 | AP13CTG12699_at | MYB family | MYB | −1.76 | −0.02 | −1.19 |
| | (Gene: SEQ ID NO: 3, | transcription | | (Down) | | (Down) |
| | Protein: SEQ ID NO: 291) | factor, putative, | | | | |
| | | expressed | | | | |
| dTF4 | Pavirv00024770m | bZIP | bZIP | −1.42 | 1.25 | 1.15 |
| | (Gene: SEQ ID NO: 4, | transcription | | (Down) | | |
| | Protein: SEQ ID NO: 292) | factor domain | | | | |
| | | containing | | | | |
| | | protein, expressed | | | | |
| dTF5 | Pavirv00012672m | MYB family | G2-like | 1.17 | −0.54 | −1.33 |
| | (Gene: SEQ ID NO: 5, | transcription | | | | (Down) |
| | Protein: SEQ ID NO: 293) | factor, putative, | | | | |
| | | expressed | | | | |
| dTF6 | Pavirv00006905m | CCT/B-box zinc | CO-like | −0.37 | 0.03 | −1.26 |
| | (Gene: SEQ ID NO: 6, | finger protein, | | | | (Down) |
| | Protein: SEQ ID NO: 294) | putative, | | | | |
| | | expressed | | | | |
| dTF7 | Pavirv000 11545m | bZIP | bZIP | −1.22 | 1.03 | −1.17 |
| | (Gene: SEQ ID NO: 7, | transcription | | (Down) | | (Down) |
| | Protein: SEQ ID NO: 295) | factor domain | | | | |
| | | containing | | | | |
| | | protein, expressed | | | | |
| dTF8 | Pavirv00039321m | dehydration- | ERF | 0.02 | −1.19 | −0.73 |
| | (Gene: SEQ ID NO: 8, | responsive | | | (Down) | |
| | Protein: SEQ ID NO: 296) | element-binding | | | | |
| | | protein, putative, | | | | |
| | | expressed | | | | |
| dTF9 | Pavirv00007251m | homeobox | HD-ZIP | −0.12 | −1.15 | −1.19 |
| | (Gene: SEQ ID NO: 9, | associated leucine | | | (Down) | (Down) |
| | Protein: SEQ ID NO: 297) | zipper, putative, | | | | |
| | | expressed | | | | |
| dTF10 | AP13ITG41879_s_at | HSF-type DNA- | HSF | −0.46 | −1.17 | −1.17 |
| | (Gene: SEQ ID NO: 10, | binding domain | | | (Down) | (Down) |
| | Protein: SEQ ID NO: 298) | containing | | | | |
| | | protein, expressed | | | | |
| dTF11 | Pavirv00007239m | bZIP | bZIP | −0.12 | 1.54 | −1.20 |
| | (Gene: SEQ ID NO: 11, | transcription | | | | (Down) |
| | Protein: SEQ ID NO: 299) | factor domain | | | | |
| | | containing | | | | |
| | | protein, expressed | | | | |
| dTF12 | Pavirv00003464m | Myb-like DNA- | G2-like | −0.44 | 0.07 | −1.15 |
| | (Gene: SEQ ID NO: 12, | binding domain | | | | (Down) |
| | Protein: SEQ ID NO: 300) | containing | | | | |
| | | protein, putative, | | | | |
| | | expressed | | | | |
| dTF13 | Pavirv00006072m | AP2 domain | ERF | 0.14 | 1.13 | −1.22 |
| | (Gene: SEQ ID NO: 13, | containing | | | | (Down) |
| | Protein: SEQ ID NO: 301) | protein, expressed | | | | |
| dTF14 | Pavirv00000078m | MYB family | MYB | −0.90 | −0.13 | −1.12 |
| | (Gene: SEQ ID NO: 14, | transcription | | | | (Down) |
| | Protein: SEQ ID NO: 302) | factor, putative, | | | | |
| | | expressed | | | | |
| dTF15 | Pavirv00012008m | ZOS3-24 - C2H2 | C2H2 | −0.64 | −1.11 | 0.05 |
| | (Gene: SEQ ID NO: 15, | zinc finger | | | (Down) | |
| | Protein: SEQ ID NO: 303) | protein, expressed | | | | |
| dTF16 | AP13CTG14279ST_s_at | NIN, putative, | Nin-like | −0.86 | 0.27 | −1.06 |
| | (Gene: SEQ ID NO: 16, | expressed | | | | (Down) |
| | Protein: SEQ ID NO: 304) | | | | | |
| dTF17 | Pavirv00053825m | heat stress | HSF | −0.13 | −1.04 | −0.89 |
| | (Gene: SEQ ID NO: 17, | transcription | | | (Down) | |
| | Protein: SEQ ID NO: 305) | factor, putative, | | | | |
| | | expressed | | | | |
| dTF18 | Pavirv00008285m | CCT/B-box zinc | CO-like | −0.55 | −1.00 | −1.03 |
| | (Gene: SEQ ID NO: 18, | finger protein, | | | (Down) | (Down) |
| | Protein: SEQ ID NO: 306) | putative, | | | | |
| | | expressed | | | | |
| dTF19 | Pavirv00010659m | OsMADS56 - | MIKC | −1.02 | 1.39 | 0.51 |
| | (Gene: SEQ ID NO: 19, | MADS-box | | (Down) | | |
| | Protein: SEQ ID NO: 307) | family gene with | | | | |
| | | MIKCc type-box, | | | | |
| | | expressed | | | | |
| dTF20 | Pavirv00067953m | auxin response | ARF | −1.00 | 0.04 | 0.58 |
| | (Gene: SEQ ID NO: 20, | factor 14, | | (Down) | | |
| | Protein: SEQ ID NO: 308) | putative, | | | | |
| | | expressed | | | | |
| dTF21 | Pavirv00005696m | basic helix-loop- | bHLH | 0.15 | 1.05 | −1.21 |
| | (Gene: SEQ ID NO: 21, | helix family | | | | (Down) |
| | Protein: SEQ ID NO: 309) | protein, putative, | | | | |
| | | expressed | | | | |
| dTF22 | Pavirv00012971m | DUF260 domain | LBD | −1.19 | −1.39 | −1.09 |
| | (Gene: SEQ ID NO: 22, | containing | | (Down) | (Down) | (Down) |
| | Protein: SEQ ID NO: 310) | protein, putative, | | | | |
| | | expressed | | | | |
| dTF59 | Pavirv00056268m | transcription | bHLH | 0.27 | 0.26 | −1.19 |
| | (Gene: SEQ ID NO: 23, | factor BIM2, | | | | (Down) |
| | Protein: SEQ ID NO: 311) | putative, | | | | |
| | | expressed | | | | |
| dTF60 | Pavirv00036358m | AP2 domain | ERF | −0.88 | −0.51 | −1.19 |
| | (Gene: SEQ ID NO: 24, | containing | | | | (Down) |
| | Protein: SEQ ID NO: 312) | protein, expressed | | | | |

¹Switchgrass gene IDs were assigned based on Phytozome v.10.1.
²Label of down regulation is only entered where the log2 fold change in expression is ≤−1.

### Example 2. Functional Characterization of dTF22 by Overexpression of its Coding Sequence in Switchgrass

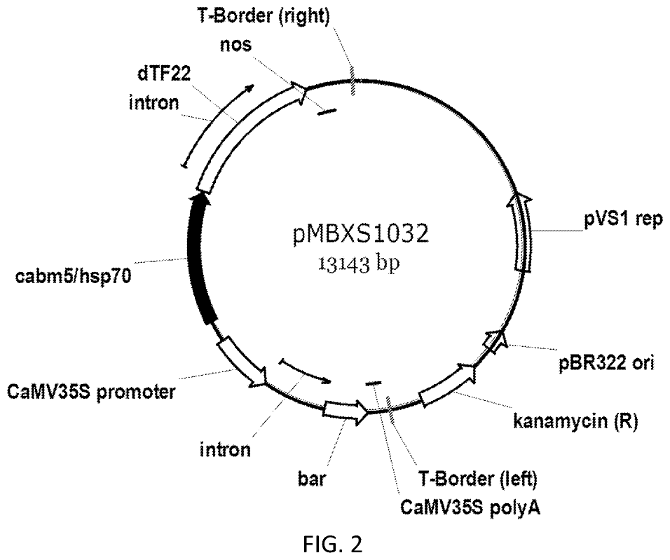

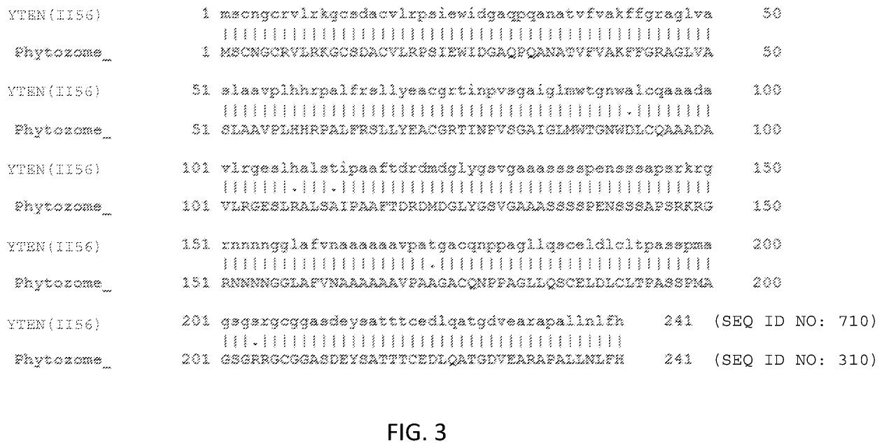

To validate the functional phenotype of dTF22, a binary vector, pMBX1032 (FIG. 2, SEQ ID NO: 25), was produced that expressed dTF22 from the maize chlorophyll a/b-binding protein promoter (Sullivan et al., 1989, Mol. Gen. Genet. 215, 431-440). This promoter is equivalent to the cab-m5 promoter described in later work (Becker et al., 1992, Plant Mol. Biol. 20, 49-60). The cab-m5 promoter is fused to the hsp70 intron (Brown and Santino, 1997, U.S. patent Ser. No. 05/593,874) for enhanced expression in monocots. The dTF22 gene used in the expression construct was amplified from genomic DNA from switchgrass genotype YTEN(II56) and contains the native intron. Alignment of the amino acid sequence of the gene isolated from YTEN(II56) to the switchgrass sequence in Phytozome showed differences in five amino acids, likely due to genotype used for isolation of the gene (FIG. 3). Immature inflorescence-derived cultures of switchgrass that were produced according to Somleva and Ali (US Patent Application 2010/0229256) were used for Agrobacterium-mediated transformation [Somleva M. N. (2006) Switchgrass (Panicum virgatum L.). In: Wang K. (eds) Agrobacterium Protocols Volume 2. Methods in Molecular Biology, vol 344. Humana Press] using the vector encoded bar gene to screen plants that were resistant to the herbicide bialophos. Primary transformants were grown under greenhouse conditions to validate the functional phenotypes. In total, 266 primary transformants representing 34 independent transformation events were obtained. Forty-one plants (14 lines, 2-3 plants/line) and 4 wild-type control plants were grown in soil for 4 months.

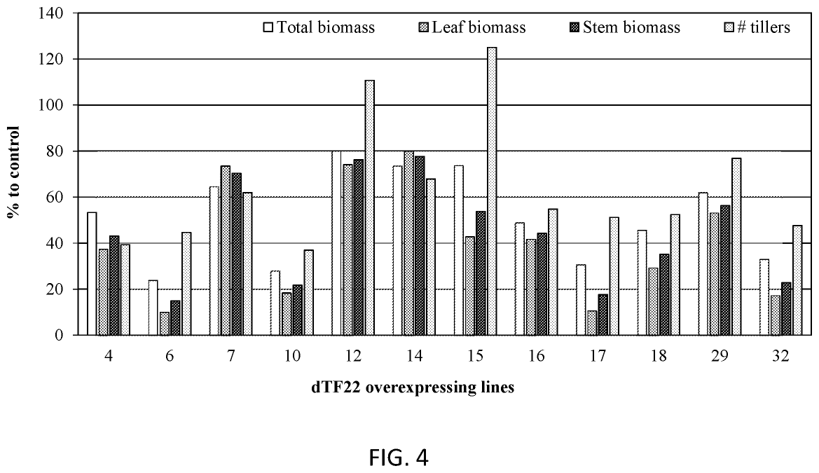

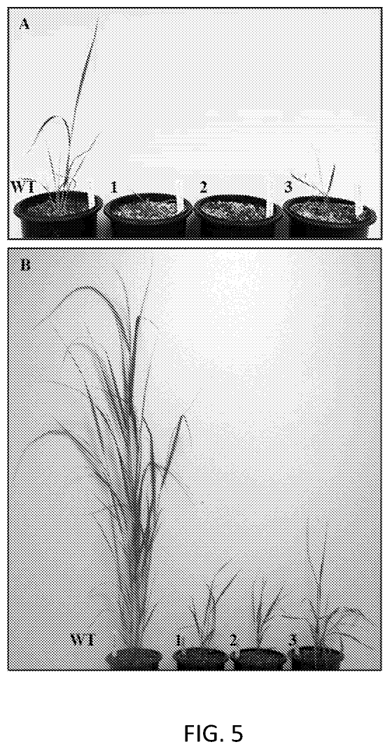

The effects of the overexpression of dTF22 on plant growth and development was evaluated by monitoring plant phenotypes in tissue culture and soil. Biomass measurements (total biomass, leaf biomass, stem biomass, and number of tillers) were obtained after growth in the greenhouse for 4 months. All vegetative and reproductive tillers at different developmental stages from each plant were counted and cut below the basal node. Leaves and stem tissues were separated, cut into smaller pieces, air-dried at 27° C. for 12-14 days and dry weight measurements were obtained. The total biomass yield of the dTF22 overexpressing lines was reduced by 20-76% compared to the wild type control (FIG. 4). No formation of reproductive tillers was detected in 12 plants representing 7 independent transformation events. A lack of normal stem formation and/or elongation was also observed in 11 out of 12 lines analyzed. FIG. 5 shows a picture of plant line 17, which was severely stunted compared to a wild-type control plant.

The dTF22 gene was found to belong to DUF, or Domain of Unknown Function, which are uncharacterized protein families that do not include any protein with known functions. Our in silico analysis of functional association networks (STRING database; https://string-db.org) showed that DUF genes are co-expressed with various abiotic stress and phytohormone related genes and pathways. The fact that overexpression of dTF22 in switchgrass yielded plants with retarded growth and delayed flowering, indicates that it is a powerful negative regulator for vegetative and reproductive development, possibly by down regulating the ABA-responsive genes, and thus is a good target for genome editing to reduce or eliminate the expression of the gene.

### Example 3. Identification of Orthologs of dTF22 in Rice and Modification or Inactivation of dTF22 Expression Using CRISPR/Cas9 Genome Editing

The switchgrass gene was used to identify the rice ortholog of dTF22 as follows. The switchgrass amino acid sequence of dTF22 (SEQ ID NO: 310) was used as a query against the rice proteome using the BLASTP search (http://rice.plantbiology.msu.edu/analyses_search_blast.shtml). The hits were ranked in order of the alignment score and the top hit, LOC_Os03g41330 (Gene: SEQ ID NO: 210, Protein: SEQ ID NO: 465), was identified as the best ortholog. It will be apparent to those skilled in the art to target the additional orthologs of SEQ ID NO: 210 for reduced expression and this is included in the scope of this invention.

For CRISPR/Cas9 genome editing of the rice dTF22, seven sgRNA sequences were designed to target various regions of the promoter and or coding sequence of the gene to either reduce expression or to inactivate the rice dTF22 gene. SEQ ID NO: 27 was used for this purpose and includes the 5′ UTR region upstream of the ATG (predicted by Phytozome and/or transcript analysis), 1000 bp of promoter sequence upstream of the 5′ UTR, and the coding sequence of the rice dTF22 gene including any introns. Guide target sequences for these sgRNAs were designed following the SpCas9 guide RNA architectures (20 nucleotides followed by a PAM sequence of NGG) using a web-based guide RNA design tool, CRISPOR, on the TEFOR website. A number of other web-based tools can also be used for guide target sequence selection and analysis, such as CRISPRdirect and CRISPR-P 2.0 (Ding et al., 2016, Frontiers in Plant Science, 7, 703; Naito et al., 2015, Bioinformatics, 31, 1120; Liu et al., 2017, Molecular Plant, 10, 530).

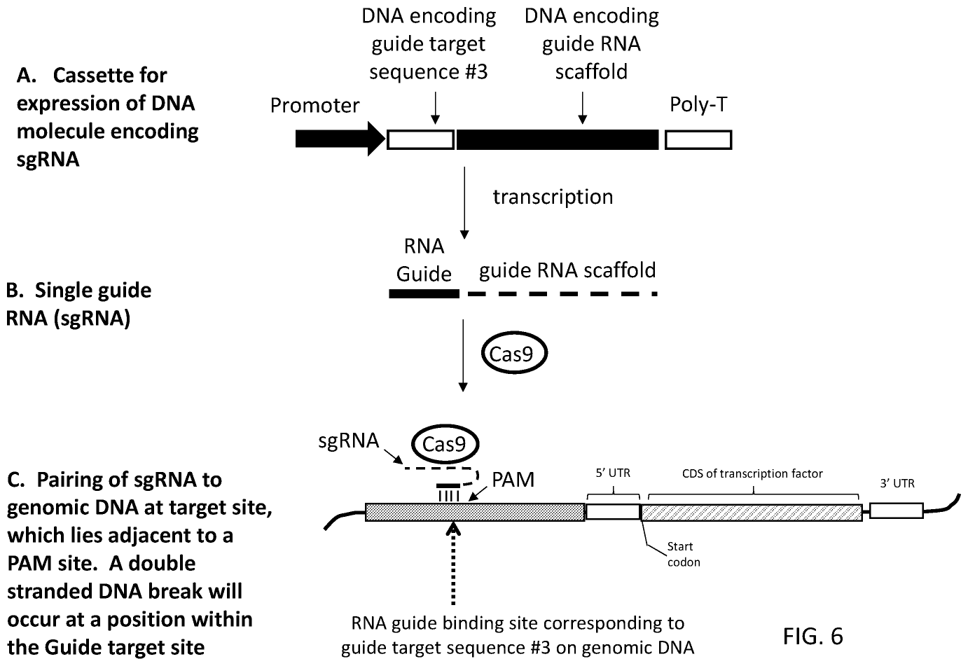

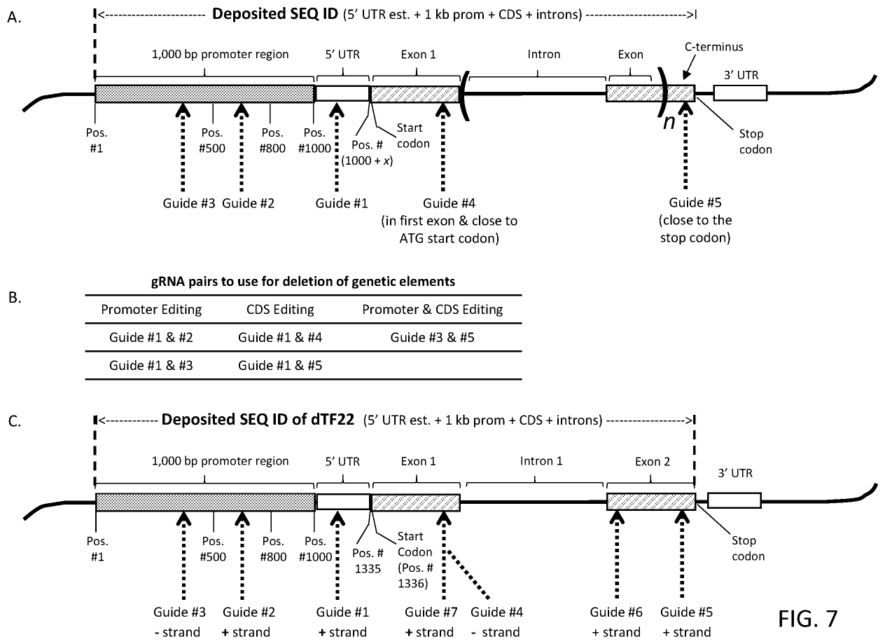

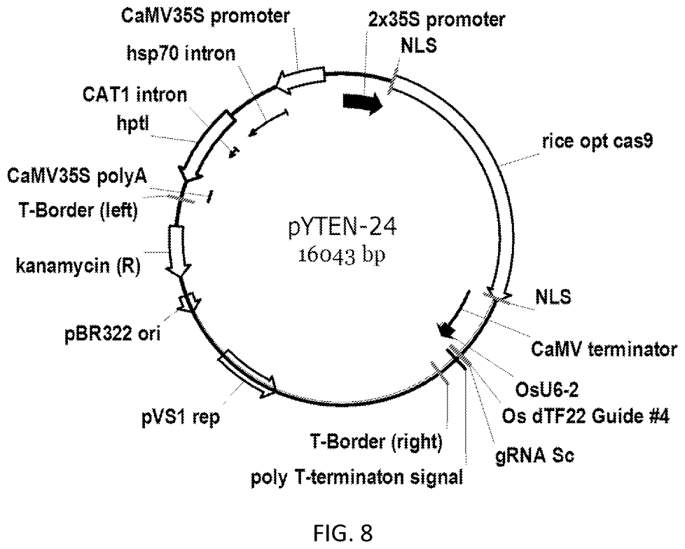

Guide target sequences 1, 2, and 3 in Table 2 target the promoter and 5′UTR region to modify expression of the dTF22 gene according to the strategies outlined in FIGS. 6 and 7. Guide target sequences 4 and 5 (Table 2, FIG. 7) as well as Guide target sequences 6 and 7 (Table 3, FIG. 7) target the coding region of the dTF22 gene. The gRNAs can be used individually to create INDELS in the promoter region. For example binary vector pYTEN-24 (FIG. 8, SEQ ID NO: 26) contains an expression cassette for Guide target sequence #4 to create an INDEL in the dTF22 gene CDS. In this vector, Guide target sequence #4 and its associated scaffold are expressed from the rice U6 promoter. The vector also contains the Cas9 enzyme codon optimized for rice expressed from the 2×355 promoter, and the hpt1 gene (containing a CAT-1 intron) for selection of transformants with hygromycin expressed from the CaMV35S promoter fused to an hsp70 intron.

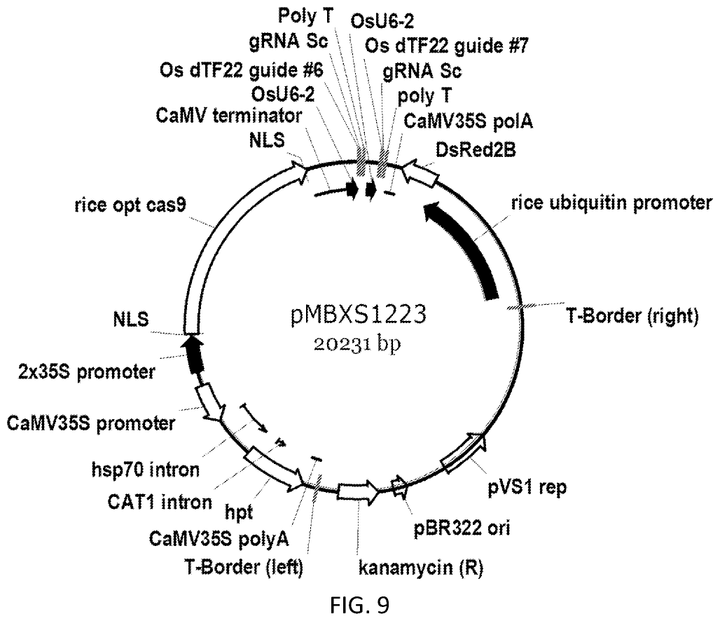

Binary vector pMBXS1223 (FIG. 9, SEQ ID NO: 543) contains expression cassettes for Guide target sequences 6 and 7 (Table 3) targeting two regions in the coding region of the dTF22 gene. The 2.504 kb rice dTF22 gene contains a large 1.804 kb intron. Guide target sequence #7 targets a site upstream of this intron while Guide target sequence #6 targets a site downstream of the intron (FIG. 7C). In vector pMBXS1223, both Guide target sequence #6 and #7, as well as their associated scaffolds, are expressed from the rice U6 promoter. The vector also contains the Cas9 enzyme codon optimized for rice expressed from the 2×35S promoter, and the hpt1 gene (containing a CAT-1 intron) for selection of transformants with hygromycin expressed from the CaMV35S promoter fused to an hsp70 intron.

TABLE 2
Guide target sequences for Cas9 mediated double
stranded cleavage of dTF22 in rice

| | | | Guide target #1 | | | Guide target #2 | | | Guide target #3 | | |
|---|---|---|---|---|---|---|---|---|---|---|---|
| | | | | Guide | | | | | | Guide | |
| | | | | target | | | Guide | | | target | |
| | | Sequence | | sequence | | | target | | | sequence | |
| | Rice | size¹, | | (5′ to | | | sequence | | | (5′ to | |
| Gene | ortholog | bp | Strand² | 3′) | PAM³ | Strand | (5′ to 3′) | PAM | Strand | 3′) | PAM |
| dTF22 | LOC_Os03g41330 | 3839 | + | TAGCTAG | GGG | + | TAAGAC | TGG | − | AACTCTC | TGG |
| | | (SEQ ID | | GTAGCTG | | | GGACAG | | | TATTAAG | |
| | | NO: 27) | | GGTATT | | | TTAAACAT | | | GGAATT | |
| | | | | (SEQ ID | | | (SEQ ID | | | (SEQ ID | |
| | | | | NO: 548) | | | NO: 549) | | | NO: 550) | |

| | | | Guide target #4 | | | Guide target #5 | | |
|---|---|---|---|---|---|---|---|---|
| | | | | Guide | | | Guide | |
| | | | | target | | | target | |
| | | Sequence | | sequence | | | sequence | |
| | Rice | size¹, | | (5′ to | | | (5′ to | |
| Gene | ortholog | bp | Strand | 3′) | PAM | Strand | 3′) | PAM |
| dTF22 | LOC_Os03g41330 | 3839 | − | CCGTCGA | CGG | + | ACGTGCG | CGG |
| | | (SEQ ID | | TCCACTC | | | AGGAAGC | |
| | | NO: 27) | | GATGCT | | | CAGCGA | |
| | | | | (SEQ ID | | | (SEQ ID | |
| | | | | NO: 551) | | | NO: 552) | |

¹Sequence includes the 5′ UTR region upstream of the ATG (predicted by Phytozome and/or transcript analysis), 1000 bp of promoter sequence upstream of the 5′ UTR, and the coding sequence of the gene including any introns.
²Strand (+/−) refers to the gRNA binding to either the forward strand of DNA (+) or its reverse complement (−).
³PAM refers to the protospacer adjacent motif for the CAS9 enzyme that resides directly adjacent to the 3′ end of the guide RNA target.

TABLE 3
Guide target sequences for Cas9 mediated double stranded
cleavage of dTF22 in rice

| | | | Guide target #6 | | | Guide target #7 | | |
|---|---|---|---|---|---|---|---|---|
| | | | | Guide | | | Guide | |
| | | Sequence | | target | | | target | |
| | Rice | size¹, | | sequence | | | sequence | |
| Gene | ortholog | bp | Strand² | (5′ to 3′) | PAM | Strand | (5′ to 3′) | PAM |
| dTF22 | LOC_Os03g41330 | 3839 | + | GCGGCG | TGG | + | TGCTGCG | TGG |
| | | (SEQ ID | | CCATCGG | | | GCCGAG | |
| | | NO: 27) | | GCTCATG | | | CATCGAG | |
| | | | | (SEQ ID | | | (SEQ ID | |
| | | | | NO: 553) | | | NO: 554) | |

¹Sequence includes the 5′ UTR region upstream of the ATG (predicted by Phytozome and/or transcript analysis), 1000 bp of promoter sequence upstream of the 5′ UTR, and the coding sequence of the gene including any introns.
²Strand (+/−) refers to the gRNA binding to either the forward strand of DNA (+) or its reverse complement (−).
³PAM refers to the protospacer adjacent motif for the CAS9 enzyme that resides directly adjacent to the 3′ end of the guide RNA target.

CRISPR Cas9 can be performed multiple ways: by introducing a complex of the Cas9 enzyme and the gRNAs (called ribonucleoprotein complexes, or RNPs) directly to protoplasts (Woo et al., 2015, 33, 1162-1164); by transfection of protoplasts either stably or transiently with a genetic construct(s) containing expression cassettes for the gRNA(s) and the Cas9 enzyme; through particle bombardment of the plant or plant tissues with a genetic construct(s) with expression cassettes for the gRNA(s) and the Cas9 enzyme; or through Agrobacterium-mediated transformation of the plant or plant tissues using a binary construct(s) with expression cassettes for the gRNA(s) and the Cas9 enzyme. An advantage of RNPs, as well as the transient expression of the expression cassettes encoding the Cas9 enzyme and the gRNAs, in protoplasts is that DNA does not stably integrate into the genome and thus does not need to be removed through segregation to produce a plant containing only the edit. For stable transformation methods, segregation of the unwanted DNA encoding the CRISPR editing machinery must be removed after the edit is obtained by conventional breeding methods.

Several methods can be used to transform rice including transformation of protoplasts (Hayashimoto, A., Z. Li, and N. Murai, A Polyethylene Glycol-Mediated Protoplast Transformation System for Production of Fertile Transgenic Rice Plants. Plant Physiology, 1990. 93(3): p. 857-863) and Agrobacterium-mediated transformation. Agrobacterium-mediated transformation of the CRISPR/Cas9 machinery for editing targeting dTF22 is described below as an example, however those skilled in the art will understand that transient or stable transformation of protoplasts, or use of RNPs with protoplasts, followed by production of callus and regeneration of plants can also be used to generate edited plants.

To initiate CRISPR/Cas9 genome editing of dTF22, binary vector pYTEN-24 is transformed into rice as follows. In preparation for rice transformation, callus of the rice cultivar Nipponbare is initiated from mature, dehusked, surface sterilized seeds on N6-basal salt callus induction media (N6-CI; contains per liter 3.9 g CHU (N₆) basal salt mix [Sigma Catalog # C1416]; 10 ml of 100× N6-vitamins [contains in final volume of 500 mL, 100 mg glycine, 25 mg nicotinic acid, 25 mg pyridoxine hydrochloride and 50 mg thiamin hydrochloride]; 0.1 g myo-inositol; 0.3 g casamino acid (casein hydrolysate); 2.88 g proline; 10 ml of 100× 2,4-dichlorophenoxyacetic acid (2,4-D), 30 g sucrose, pH 5.8 with 4 g gelrite or phytagel). Approximately 100 seeds are used for each transformation. The frequency of callus induction is scored after 21 days of culture in the dark at 27±1° C. Callus induction from the scutellum with a high frequency (of about 96% total callus induction) is observed.

Rice transformation vector pYTEN-24 is transformed into Agrobacterium strain AGL1. The resulting Agrobacterium strain containing the vector is resuspended in 10 mL of MG/L medium (5 g tryptone, 2.4 g yeast extract, 5 g mannitol, 5 g Mg₂SO₄, 0.25 g K₂HPO₄, 1 g glutamic acid and 1 g NaCl) to a final OD600 of 0.3. Approximately twenty-one day old scutellar embryogenic callus are cut to about 2-3 mm in size and are infected with Agrobacterium containing pYTEN-24 for 5 min. After infection, the calli are blotted dry on sterile filter papers and transferred onto co-cultivation media (N6-CC; contains per liter 3.9 g CHU (N₆) basal salt mix; 10 ml of 100× N6-vitamins; 0.1 g myo-inositol; 0.3 g casamino acid; 10 ml of 100× 2,4-D, 30 g sucrose, 10 g glucose, pH 5.2 with 4 g gelrite or phytagel and 1 mL of acetosyringone [19.6 mg/mL stock]). Co-cultivated calli are incubated in the dark for 3 days at 25° C. After three days of co-cultivation, the calli are washed thoroughly in sterile distilled water to remove the bacteria. A final wash with a timentin solution (250 mg/L) is performed and calli are blotted dry on sterile filter paper. Callus are transferred to selection media [N6-SH; contains per liter 3.9 g CHU (N₆) basal salt mix, 10 ml of 100× N6-vitamins, 0.1 g myo-inositol, 0.3 g casamino acid, 2.88 g proline, 10 ml of 100× 2,4-D, 30 g sucrose, pH 5.8 with 4 g phytagel and 500 μL of hygromycin (stock concentration: 100 mg/ml)] and incubated in the dark for two-weeks at 27±1° C. The transformed calli that survive the selection pressure and that proliferate on N6-SH medium are sub-cultured on the same media for a second round of selection. These calli are maintained under the same growth conditions for another two-weeks. The number of plants regenerated after 30 days on N6-SH medium is scored and the frequency calculated. After 30 days, the proliferating calli are transferred to regeneration media (N6-RH medium; contains per liter 4.6 g MS salt mixture, 10 ml of 100× MS-vitamins [MS-vitamins contains in 500 mL final volume 250 mg nicotinic acid, 500 mg pyridoxine hydrochloride, 500 mg thiamine hydrochloride, 100 mg glycine], 0.1 g myo-inositol, 2 g casein hydrolysate, 1 ml of 1,000×1-naphtylacetic acid solution [NAA; contains in 200 mL final volume 40 mg NAA and 3 mL of 0.1 N NaOH], 20 ml of 50× kinetin [contains in 500 mL final volume 50 mg kinetin and 20 mL 0.1 N HCl], 30 g sucrose, 30 g sorbitol, pH 5.8 with 4 g phytagel and 500 μl of a 100 mg/mL hygromycin stock). The regeneration of plantlets from these calli occurs after about 4-6 weeks. Rooted plants are transferred into peat-pellets for one week to allow for hardening of the roots. The plants are then kept in zip-loc bags for acclimatization. Plants (T0 generation) are transferred into pots and grown in a greenhouse.

The T0 plants are examined for edits as follows: During growth, leaf material from the T0 transformants is harvested and DNA is extracted from the plant tissue using a Qiagen Plant DNeasy kit. PCR reactions are performed using primers that bind to regions of genomic DNA about 100 base pairs away from the gRNA binding site. Sequencing analysis is performed on the crude PCR mixture using a Next-Generation sequencing technology and automated sequencing assembly offered by a vendor. Plants with INDELS are identified and allowed to grow in a greenhouse to maturity prior to seed harvest (T1 generation).

T1 seeds are planted and grown in a greenhouse, leaf tissue is harvested, and genomic DNA is isolated. Lines are screened for the presence of the hpt1 gene or the Cas9 gene by PCR. Plants that no longer have these genes may have lost the DNA encoding the Cas9 machinery but may still retain the edit. Screening can also be done by co-expressing a visual marker such as DsRed, a red fluorescent protein from the Discoma genus of coral (Matz et al., 1999, Nat. Biotechnol. 17, 969-973), by placing an expression cassette coding the gene on vector pYTEN-24 to allow visual detection of seeds that no longer carry the vector encoded transgenes. Ti transgene free plants are thus further screened for edits by extracting genomic DNA from leaf tissue and performing PCR reactions using primers that bind to regions of genomic DNA about 100 base pairs away from the gRNA binding site. Sequencing analysis is performed on the crude PCR mixture using a Next-Generation sequencing technology and automated sequencing assembly offered by a vendor. Plants with INDELS are identified. The sequence of the edits is analyzed and edits that insert 1 base or that delete 1, 2, 4, 5, 7, 8 or more bases are selected. These INDELS will create a reading frame shift likely creating a truncated protein. Lines with the best INDELS are allowed to grow in a greenhouse to maturity prior to seed harvest (T2 generation). The expression levels of dTF22 in various tissues of rice is determined. Transcript levels of leaves, stem tissues, panicles and seeds at different developmental stages are determined by RT-PCR using a gene such as β-actin as a reference. Total RNA is isolated from the different rice tissues using the RNeasy Plant Mini Kit (Qiagen, Valencia, Calif., USA) according to the manufacturer's protocol. DNase treatment and column purification are performed and RNA quality is assessed using an Agilent 2100 Bioanalyzer (Agilent Technologies, Santa Clara, Calif., USA) according to the manufacturer's instructions. The RT-PCR analysis is performed with 50 ng of total RNA using a One Step RT-PCR Kit (Qiagen, Valencia, Calif., USA). Lines with reduced expression of dTF22 are evaluated.

If required, lines can be grown another generation to obtain homogenous edits.

Rice lines are evaluated for their total grain yield and other agronomic parameters such as drought tolerance, stress tolerance, stem thickness, number of tillers, size of panicle and 100 seed weight of the rice grains can also be analyzed. High yielding lines or lines with good agronomic parameters indicating improved performance as compared the control plants where the expression of dTF22 has not been reduced are advanced.

INDELS can be made in just the promoter region to reduce expression of dTF22 by modifying vector pYTEN-24 to contain either Guide target sequences #1, 2, or 3 (Table 2, FIG. 7C). Transformation of rice and screening for edits is performed as previously described and rice lines can be screened for reduced expression of dTF22 and/or increased yield.

Alternatively, two of the gRNAs can be co-expressed, as described in FIG. 7B, to excise a larger piece of DNA from the coding sequence of dTF22, it's promoter region, or both. To excise DNA from the CDS, guide target sequences can be selected from numbers 4, 5, 6, and 7 (Tables 2 and 3). To excise the majority of the promoter and the CDS, Guide target sequences 3 and 5 can be used. To excise DNA only within the promoter region, guide target sequences can be selected from 1, 2, and 3.

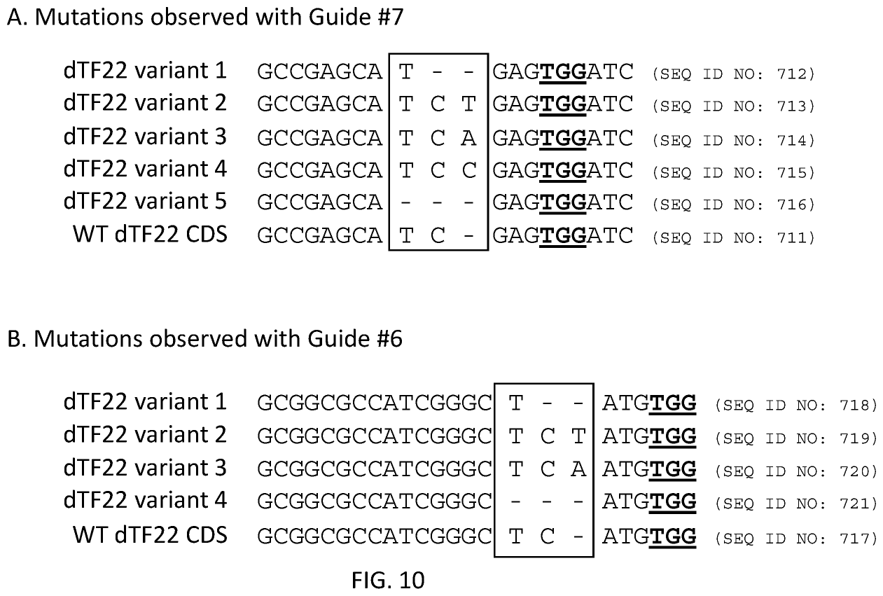

To generate edits in dTF22 with Guide target sequences #6 and #7, callus of the rice cultivar Nipponbare was transformed with vector pMBXS1223 (FIG. 9) using Agrobacterium strain AGL1 as described above. A total of 34 putative rice T0 transgenic plants from five different transformed callus lines were obtained. Transgenic lines were screened by PCR for the presence of the Cas9 gene and two of the 34 putative rice T0 transgenic plants did not contain Cas9 and were discarded. Genomic DNA was isolated from plants during the early vegetative growth stage (approximately 3-4 weeks after transfer to soil) using the Qiagen Plant DNeasy extraction kit. Tissue was sampled from young leaves and flash frozen with liquid nitrogen prior to DNA extraction. Edits were characterized by amplicon sequencing using an outside vendor. Two of the plants had ambiguous sequencing results and were discarded. The remaining 30 plants are shown in Table 4. For Guide target sequence 7, five different types of edits were observed (FIG. 10A) and some lines contained more than one edit. A summary of sequencing results for the individual plants observed for Guide target sequence #7 is listed in Table 4. All mutations result in a change in reading frame for the protein which result in truncated proteins of varying length depending on the mutant. Plants with Variant 2 are the most desired of the variants obtained since Variant 2 produces a short truncated protein of only 23 amino acids.

Only a few lines were analyzed for edits with Guide target sequence #6. For these lines, 4 different types of edits were observed (FIG. 10B). A summary of sequencing results for the individual plants observed for Guide target sequence #6 is listed in Table 4.

TABLE 4
Rice plants with confirmed edits

| | |
|---|---|
| Guide target #7 | Guide target #6 |

| | | % of | | % of |
|---|---|---|---|---|
| Plant | Variant | variant | Variant | variant |
| ID | number¹ | reads² | number³ | reads² |
| 16 | 1 | 97.08% | N.A. | N.A. |
| 10a | 2 | 98.89% | 3 | 46.26 |
| | | | 4 | 43.61 |
| 10b | 2 | 99.12% | N.A. | N.A. |
| 10c | 2 | 99.33% | N.A. | N.A. |
| 10d | 2 | 98.24% | 3 | 46.25 |
| | | | 4 | 44.79 |
| 10e | 2 | 98.93% | N.A. | N.A. |
| 10f | 2 | 100% | 3 | 39.32 |
| | | | 4 | 37.24 |
| 10g | 2 | 100% | N.A. | N.A. |
| 17b | 2 | 49.34% | N.A. | N.A. |
| 17b | 4 | 47.34% | | |
| 32a | 2 | 95.25% | N.A. | N.A. |
| 32c | 3 | 49.36% | N.A. | N.A. |
| | 4 | 48.43% | | |
| 32d | 3 | 48.07% | N.A. | N.A. |
| | 4 | 47.60% | | |
| 32e | 3 | 49.43% | N.A. | N.A. |
| | 4 | 48.18% | | |
| 32f | 2 | 41.75% | N.A. | N.A. |
| | 5 | 47.33% | | |
| 32g | 3 | 49.94% | N.A. | N.A. |
| | 4 | 48.38% | | |
| 32h | 3 | 47.06% | N.A. | N.A. |
| | 4 | 45.74% | | |
| 32j | 2 | 49% | N.A. | N.A. |
| | 5 | 50.35% | | |
| 321 | 2 | 95.30% | N.A. | N.A. |
| 32n | 2 | 96.06% | N.A. | N.A. |
| 32o | 2 | 96.17% | N.A. | N.A. |
| 32p | 1 | 50.13% | N.A. | N.A. |
| | 4 | 48.83% | | |
| 32q | 1 | 51.70% | N.A. | N.A. |
| | 4 | 47.89% | | |
| 4i | 1 | 99.48% | 1 | 46.47 |
| | | | 2 | 45.94 |
| 4j | 1 | 98.76% | N.A. | N.A. |
| 4k | 1 | 99.38% | 1 | 48.05 |
| | | | 2 | 47.56 |
| 4m | 1 | 97.95% | N.A. | N.A. |
| 4n | 1 | 98.34% | 1 | 45.54 |
| | | | 2 | 47.28 |
| 4o | 1 | 96.36% | 1 | 46.58 |
| | | | 2 | 47.88 |
| 4p11 | 1 | 98.46% | 1 | 44.49 |
| | | | 2 | 46.04 |
| 4p12 | 1 | 99.09% | 1 | 47.66 |
| | | | 2 | 48.43 |
| WT-7 | wt | 98.81% | | |
| control | | | | |
| WT-8 | wt | 99.03% | | |
| control | | | | |

WT-7 and WT-8 are wild-type control plants. N.A., sample not analyzed.
¹Variant number refers to the sequence of the edit for Guide target #7 described in FIG. 10A.
²Percent of reads in amplicon sequencing data that contain the sequence of the indicated variant.
³Variant number refers to the sequence of the edit for Guide target #6 described in FIG. 10B.

T0 plants with edits were grown to produce seed and T1 seed was harvested. The presence of an expression cassette with the DsRed protein on vector pMBXS1223 allows seeds to be screened for the presence of vector DNA. A portion of the T1 seeds will be expected to retain the edit but lose the vector DNA encoding the CRISPR/Cas9 editing machinery via segregation. DsRed negative seeds from line 10f (Table 4) were dehusked, sterilized, and placed in sterile petri dishes with filter paper and 3 mL of sterile water. Seeds were germinated in the growth chamber at 28° C. with a 16 h light and 8 h dark cycle. Improved germination of the edited line 10f was observed. Germination was monitored at 2 days and 6 days. Of 64 total seeds analyzed from edited line 10f, 28 germinated within two days (44% germination). Out of 59 wild-type seed, 5 germinated (8% germination) within two days. After six days, 58 of 64 seeds from edited line 10f had germinated and had shoots (91%). After six days, 13 of 59 wild-type control seeds had germinated and had shoots (22%). Germinated seeds with shoots were transferred to peat pellets. Plants are transferred to soil. The edits in the T1 leaf tissue are characterized by amplicon sequencing.

Genome editing of rice homologs to the rice dTF22 gene

In addition to editing of the rice dTF22 gene (LOC_Os03g41330, SEQ ID NO: 27) as described above, two other rice dTF22 homologous genes, LOC_Os03g33090 (SEQ ID NO: 544) and LOC_Os03g45750 (SEQ ID NO: 545) were selected for editing based on their in-silico expression profiles. Guide target sequences can be designed to edit LOC_Os03g33090 and LOC_Os03g45750. Editing of one or more genes selected from the group of LOC_Os03g41330 (SEQ ID NO: 27), LOC_Os03g33090 (SEQ ID NO: 544), and LOC_Os03g45750 (SEQ ID NO: 545) can be performed as described above.

TABLE 5
Rice dTF22 homologous genes

| Gene | Gene ID | Sequence size¹ in base pairs |
|---|---|---|
| Rice ortholog to switchgrass dTF22 | LOC_Os03g41330 | 3839 |
| (Protein: SEQ ID: 465) | | (SEQ ID NO: 27) |
| Rice gene homologous to | LOC_Os03g33090 | 1866 |
| LOC_Os03g41330 | | (SEQ ID NO: 544) |
| (Protein: SEQ ID: 546) | | |
| Rice gene homologous to | LOC_Os03g45750 | 3252 |
| LOC_Os03g41330 | | (SEQ ID NO: 545) |
| (Protein: SEQ ID: 547) | | |

¹Sequence includes the 5' UTR region upstream of the ATG (predicted by Phytozome and/or transcript analysis), 1000 bp of promoter sequence upstream of the 5' UTR, and the coding sequence of the gene including any introns.

### Example 4. Identification of Orthologs of dTF22 in Other Crops and Design of gRNAs for CRISPR/Cas9 Genome Editing

Orthologs of the switchgrass dTF22 gene were found by reciprocal BLAST searches against all the proteins encoded by the genes annotated in the genome of interest. A reciprocal blast hit is found when the proteins encoded by two genes, each in a different genome, find each other as the best scoring match. The National Center for Biotechnology Information database, The Phytozome database from the Joint Genome Institute, The Michigan State University Rice Genome Annotation Project database, and The Plant Transcription Factor Database were used for the BLAST searches and to extract the orthologous gene sequences and gene ID's.

Guide target sequences to edit the promoter region of dTF22 in various crops, using the strategy described in FIG. 7A-B, are shown in Table 6. These guide target sequences can be used to form single gRNAs to make INDELS in the promoter regions and lines can be screened for reduced expression of dTF22. Alternatively, Guide target sequences #1 and #3 can be co-expressed to delete the majority of the promoter and 5′ UTR to inactivate the promoter. Guide target sequences to edit the coding sequence as described in FIG. 7A-B are shown in Table 7 for maize, Medicago truncatula, and wheat. The genome sequence for alfalfa (Medicago sativa) is not publically available. The sequence of a close relative to alfalfa, Medicago truncatula was used to find the dTF22 ortholog. Once the genome sequence of alfalfa (Medicago sativa) is publically available, the dTF22 ortholog can be found by comparison with the Medicago truncatula and switchgrass genes. Two guide target sequences can be selected from Tables 6 and 7 to produce a sgRNA, using the procedures described in FIG. 6, and co-expressed to delete a region of the promoter and/or CDS as described in FIG. 7B.

TABLE 6
Orthologs of dTF22 in major crops and gRNAs to edit the promoter region.

| | | | Guide target #1 | | | Guide target #2 | | | Guide target #3 | | |
|---|---|---|---|---|---|---|---|---|---|---|---|
| | | | | Guide | | | Guide | | | Guide | |
| | | Sequence | | target | | | target | | | target | |
| | | size¹, | | sequence | | | sequence | | | sequence | |
| Crop | dTF22 ortholog | bp | Strand² | (5′ to 3′) | PMA³ | Strand | (5′ to 3′) | PMA | Strand | (5′ to 3′) | PMA |
| Maize | GRMZM2G017319 | 3237 | + | CATTAAACGT | AGG | + | TACGATGCAGA | GGG | − | GCTCCCGGGT | CGG |
| | (Protein: SEQ ID | (SEQ ID | | ACGAGACTGC | | | GGTGAGCTG | | | TTAATTTTCC | |
| | NO: 334) | NO: 28) | | (SEQ ID NO: | | | (SEQ ID NO: | | | (SEQ ID NO: | |
| | | | | 555) | | | 556) | | | 557) | |
| soybean | Glyma05g22120 | 1272 | + | AAGACACACT | GGG | − | CTTCTGTATTA | TGG | + | CTCTCTTTTCT | TGG |
| | (Protein: SEQ ID | (SEQ ID | | CACACACCCT | | | TTGTAAGTA | | | CTCTCATAG | |
| | NO: 417) | NO: 29) | | (SEQ ID NO: | | | (SEQ ID NO: | | | (SEQ ID NO: | |
| | | | | 558) | | | 559) | | | 560) | |
| Brassica | BnaC02g16720D | 1145 | + | AACAACTGGA | CGG | + | ACTACCCTCTC | GGG | − | TCCGATTGGG | TGG |
| napus | (Protein: SEQ ID | (SEQ ID | | CAGACTCTGT | | | TCTCTCAAA | | | ATTCTACCGT | |
| | NO: 441) | NO: 30) | | (SEQ ID NO: | | | (SEQ ID NO: | | | (SEQ ID NO: | |
| | | | | 561) | | | 562) | | | 563) | |
| Camelina | Csa18g040020 | 1156 | + | TCTTCATTCTC | AGG | − | AATAAAGAGA | GGG | + | TCTTTTTACC | AGG |
| sativa | (Protein: SEQ ID | (SEQ ID | | CAGACCCTC | | | CATAGGGTAC | | | ACTCTCTCTA | |
| | NO: 542) | NO: 31) | | (SEQ ID NO: | | | (SEQ ID NO: | | | (SEQ ID NO: | |
| | | | | 564) | | | 565) | | | 566) | |
| Sorghum | Sb01g014800 | 1357 | + | CAGCTTATAT | AGG | + | TTACCTGCGTA | GGG | − | TTTCTATTCAT | TGG |
| bicolor | (Protein: SEQ ID | (SEQ ID | | ATATCGAGAC | | | GAGGATCCT | | | TGTAGCTAG | |
| | NO: 513) | NO: 32) | | (SEQ ID NO: | | | (SEQ ID NO: | | | (SEQ ID NO: | |
| | | | | 567) | | | 568) | | | 569) | |
| Medicago | Medtr1g106420 | 1776 | − | TGTATGGTCT | AGG | − | CTCAGGTTCTC | TGG | − | AGTAAATAAG | TGG |
| truncatula | (Protein: SEQ ID | (SEQ ID | | TATATCTTGC | | | CGCAAATGT | | | CATTGGTTGT | |
| | NO: 489) | NO: 33) | | (SEQ ID NO: | | | (SEQ ID NO: | | | (SEQ ID NO: | |
| | | | | 570) | | | 571) | | | 572) | |
| wheat | Traes_4AL_7241716B6 | 3051 | − | TGGACAGTCG | CGG | + | CCTCCCCCCGA | AGG | + | GTATAACTTT | AGG |
| | (Protein: SEQ ID | (SEQ ID | | ATCACCGTAT | | | TTTCAATGG | | | AGCCAGATGG | |
| | NO: 537) | NO: 34) | | (SEQ ID NO: | | | (SEQ ID NO: | | | (SEQ ID NO: | |
| | | | | 573) | | | 574) | | | 575) | |

^{¹}For maize, Medicago truncatula, and wheat, the sequence size and SEQ ID includes the 5′ UTR region upstream of the ATG (predicted by Phytozome, MaizeGDB, GenBank, and/or transcript analysis), 1000 bp of promoter sequence upstream of the 5′ UTR, as well as the coding sequence of the gene including any introns. For soybean, Brassica napus, Camelina sativa, and sorghum bicolor, the sequence size and SEQ ID includes only the 5′ UTR region upstream of the ATG (predicted by Phytozome, GenBank, and/or transcript analysis), and 1000 bp of promoter sequence upstream of the 5′ UTR.
^{²}Strand (+/−) refers to the gRNA binding to either the forward strand of DNA (+) or its reverse complement (−).
^{³}PAM refers to the protospacer adjacent motif for the CAS9 enzyme that resides directly adjacent to the 3′ end of the guide RNA target.
^{⁴}The genome sequence for alfalfa (Medicago sativa) is not publically available. The sequence of a close relative to alfalfa, Medicago truncatula was used to find the dTF22 ortholog. Once the genome sequence of alfalfa (Medicago sativa) is publically available, the dTF22 ortholog can be found by comparison with the Medicago truncatula and switchgrass genes.

TABLE 7
Guide target sequences to edit the coding sequence of dTF22 in major crops

| | | | Guide target #4 | | | Guide target #5 | | |
|---|---|---|---|---|---|---|---|---|
| | | | | Guide | | | Guide | |
| | | Sequence | | sequence | | | sequence | |
| | | size¹, | | target | | | target | |
| Crop | dTF22 ortholog | bp | Strand² | (5′ to 3′) | PAM³ | Strand | (5′ to 3′) | PAM |
| maize | GRMZM2G017319 | 3237 | − | CGTGGATCCA | GGG | + | GAGGACCAGA | CGG |
| | (Protein: SEQ ID | (SEQ ID | | GTCGATGCTG | | | CCGGTGATCA | |
| | NO: 334) | NO: 28) | | (SEQ ID NO: | | | (SEQ ID NO: 577) | |
| | | | | 576) | | | | |
| Medicago | Medtr1g106420 | 1776 | + | TGTTACGAGA | TGG | + | GAATCGGAGTC | GGG |
| truncatula⁴ | (Protein: SEQ ID | (SEQ ID | | TTGTCTAACG | | | TTCCACGTTG | |
| | NO: 489) | NO: 33) | | (SEQ ID NO: | | | (SEQ ID NO: 579) | |
| | | | | 578) | | | | |
| wheat | Traes_4AL_7241716B6 | 3051 | − | GGGAGGCGA | CGG | + | AGCAGCAGGT | GGG |
| | (Protein: SEQ ID | (SEQ ID | | CGAGGCCGGCG | | | GAAGCTGCCG | |
| | NO: 537) | NO: 34)) | | (SEQ ID NO: | | | (SEQ ID NO: 581) | |
| | | | | 580) | | | | |

¹Sequence includes the 5′ UTR region upstream of the ATG (predicted by Phytozome, MaizeGDB, Genbank, and/or transcript analysis), 1000 bp of promoter sequence upstream of the 5′ UTR, as well as the coding sequence of the gene including any introns.
²Strand (+/−) refers to the gRNA binding to either the forward strand of DNA (+) or its reverse complement (−).
³PAM refers to the protospacer adjacent motif for the CAS9 enzyme that resides directly adjacent to the 3′ end of the guide RNA target.
⁴The sequence for alfalfa (Medicago sativa) is not publically available. The sequence of a close relative to alfalfa, Medicago truncatula was used. Once the genome sequence of alfalfa (Medicago sativa) is publically available, the dTF22 ortholog can be found by comparison with the Medicago truncatula and switchgrass genes.

Crop specific vectors or DNA fragments to edit dTF22 through stable transformation can be designed for multiple crops including maize, rice, soybean, canola, wheat, alfalfa, sorghum, and camelina. These constructs will contain the following expression cassettes: (a) an expression cassette for the Cas9 gene that contains a promoter functional in that crop, the Cas9 gene that includes nuclear localization sequences on the 5′ and 3′ end of the gene, and a terminator; (b) one or more expression cassettes for a guide RNA(s) that consists of a promoter, the guide target sequence with about 20 bp homology upstream of a PAM sequence with the consensus sequence of “NGG”, a gRNA scaffold sequence necessary for Cas9 binding, and a poly T-termination sequence (the promoter for gRNAs is preferably a U6 promoter functional in the crop to be transformed); (c) an expression cassette for a selectable marker that can be used for the specific crop for selection of transformants. For Agrobacterium-mediated transformation, these expression cassettes can be cloned into one or more binary vectors for transformation of the appropriate explant of the crop. For stable transformation by particle bombardment or protoplast transformation, expression cassettes can be introduced as a DNA fragment(s) or can be localized on one or more simple plasmid vectors. For both methods, plants can be screened for edits using Next Generation Sequencing methods. After the edits are obtained, the expression cassettes described above can be removed by segregation using conventional breeding methods for the crop.

For transient expression of protoplasts, the expression cassettes described above for the Cas9 and the gRNA can be introduced as one or more DNA fragments or can be localized on one or more simple vectors. An expression cassette for a selectable marker is not required. Protoplast cultures or alternatively, callus cultures derived from the protoplast cultures, can be screened for edits using Next Generation Sequencing methods, and protoplast or callus cultures with the edits can be regenerated into plants.

For editing using ribonucleoprotein complexes or RNPs, purified Cas9 enzyme can be mixed with one or more gRNAs to form a complex of the Cas9 enzyme and the gRNAs which can then be introduced directly to protoplasts. Protoplast cultures or alternatively, callus cultures derived from the protoplast cultures, can be screened for edits using Next Generation Sequencing methods, and protoplast or callus cultures with the edits can be regenerated into plants.

Examples of transformation methods that can be used for editing are listed below.

Maize:

For transformation of maize, a binary vector containing expression cassettes for the Cas9 gene, gRNA(s), and a selectable marker, such as the bar gene imparting resistance to the herbicide bialophos, are prepared. In preparation for transformation, the binary vector is transformed into an Agrobacterium tumefaciens strain, such as A. tumefaciens strain EHA101. Agrobacterium-mediated transformation of maize can be performed following a previously described procedure (Frame et al., 2006, Agrobacterium Protocols Wang K., ed., Vol. 1, pp 185-199, Humana Press) as follows.

Plant Material:

Plants grown in a greenhouse are used as an explant source. Ears are harvested 9-13 dafter pollination and surface sterilized with 80% ethanol.

Explant Isolation, Infection and Co-Cultivation:

Immature zygotic embryos (1.2-2.0 mm) are aseptically dissected from individual kernels and incubated in an A. tumefaciens strain EHA101 culture containing the transformation vector of interest for genome editing (grown in 5 ml N6 medium supplemented with 100 μM acetosyringone for stimulation of the bacterial vir genes for 2-5 h prior to transformation) at room temperature for 5 min. The infected embryos are transferred scutellum side up on to a co-cultivation medium (N6 agar-solidified medium containing 300 mg/l cysteine, 5 μM silver nitrate and 100 μM acetosyringone) and incubated at 20° C., in the dark for 3 d. Embryos are transferred to N6 resting medium containing 100 mg/l cefotaxime, 100 mg/l vancomycin and 5 μM silver nitrate and incubated at 28° C., in the dark for 7 d.

Callus Selection:

All embryos are transferred on to the first selection medium (the resting medium described above supplemented with 1.5 mg/l bialaphos) and incubated at 28° C. in the dark for 2 weeks followed by subculture on a selection medium containing 3 mg/l bialaphos. Proliferating pieces of callus are propagated and maintained by subculture on the same medium every 2 weeks.

Plant Regeneration and Selection:

Bialaphos-resistant embryogenic callus lines are transferred on to regeneration medium I (MS basal medium supplemented with 60 g/l sucrose, 1.5 mg/l bialaphos and 100 mg/l cefotaxime and solidified with 3 g/l Gelrite) and incubated at 25° C. in the dark for 2 to 3 weeks. Mature embryos formed during this period are transferred on to regeneration medium II (the same as regeneration medium I with 3 mg/l bialaphos) for germination in the light (25° C., 80-100 μmol/m²/s light intensity, 16/8-h photoperiod). Regenerated plants are ready for transfer to soil within 10-14 days. Plants are grown in the greenhouse to maturity and T1 seeds are isolated.

Plant tissue from the T1 and T2 generations are screened for edits using Next Generation Sequencing as previously described for rice. The T-DNA insert containing the CRISPR Cas9 editing machinery is removed from the plants, while retaining the edit, via segregation implemented through conventional breeding.

Soybean:

For transformation of soybean, a biolistic method is employed. The transformation, selection, and plant regeneration protocol for soybean is adapted from Simmonds (2003) (Simmonds, 2003, Genetic Transformation of Soybean with Biolistics. In: Jackson J F, Linskens H F (eds) Genetic Transformation of Plants. Springer Verlag, Berlin, pp 159-174) and requires expression cassettes for the Cas9 enzyme, the gRNA(s), and a selectable marker, such as the hygromycin resistance marker. These expression cassettes can be co-localized on one plasmid or isolated DNA fragment, or alternatively, two separate plasmids or isolated DNA fragments containing the expression cassettes can be co-bombarded.

The purified DNA fragment(s) are introduced into embryogenic cultures of soybean Glycine max cultivars X5 and Westag97 via biolistics, to obtain transgenic plants. The transformation, selection, and plant regeneration of soybean is performed as follows.

Induction and Maintenance of Proliferative Embryogenic Cultures:

Immature pods, containing 3-5 mm long embryos, are harvested from host plants grown at 28/24° C. (day/night), 15-h photoperiod at a light intensity of 300-400 μmol m⁻² s⁻¹. Pods are sterilized for 30 s in 70% ethanol followed by 15 min in 1% sodium hypochlorite [with 1-2 drops of Tween 20 (Sigma, Oakville, ON, Canada)] and three rinses in sterile water. The embryonic axis is excised and explants are cultured with the abaxial surface in contact with the induction medium [MS salts, B5 vitamins (Gamborg O L, Miller R A, Ojima K. Exp Cell Res 50:151-158), 3% sucrose, 0.5 mg/L BA, pH 5.8), 1.25-3.5% glucose (concentration varies with genotype), 20 mg/l 2,4-D, pH 5.7]. The explants, maintained at 20° C. at a 20-h photoperiod under cool white fluorescent lights at 35-75 μmol m⁻² s⁻¹, are sub-cultured four times at 2-week intervals. Embryogenic clusters, observed after 3-8 weeks of culture depending on the genotype, are transferred to 125-ml Erlenmeyer flasks containing 30 ml of embryo proliferation medium containing 5 mM asparagine, 1-2.4% sucrose (concentration is genotype dependent), 10 mg/l 2,4-D, pH 5.0 and cultured as above at 35-60 μmol m⁻² s⁻¹ of light on a rotary shaker at 125 rpm. Embryogenic tissue (30-60 mg) is selected, using an inverted microscope, for subculture every 4-5 weeks.

Transformation: Cultures are bombarded 3 days after subculture. The embryogenic clusters are blotted on sterile Whatman filter paper to remove the liquid medium, placed inside a 10×30-mm Petri dish on a 2×2 cm² tissue holder (PeCap, 1 005 μm pore size, Band SH Thompson and Co. Ltd. Scarborough, ON, Canada) and covered with a second tissue holder that is then gently pressed down to hold the clusters in place. Immediately before the first bombardment, the tissue is air dried in the laminar air flow hood with the Petri dish cover off for no longer than 5 min. The tissue is turned over, dried as before, bombarded on the second side and returned to the culture flask. The bombardment conditions used for the Biolistic PDS-I000/He Particle Delivery System are as follows: 737 mm Hg chamber vacuum pressure, 13 mm distance between rupture disc (Bio-Rad Laboratories Ltd., Mississauga, ON, Canada) and macrocarrier. The first bombardment uses 900 psi rupture discs and a microcarrier flight distance of 8.2 cm, and the second bombardment uses 1100 psi rupture discs and 11.4 cm microcarrier flight distance. DNA precipitation onto 1.0 μm diameter gold particles is carried out as follows: 2.5 μl of 100 ng/μl of insert DNA (Cas9 and gRNA(s) expression cassettes) and 2.5 μl of 100 ng/μl selectable marker DNA (cassette for hygromycin selection) are added to 3 mg gold particles suspended in 50 μl sterile dH₂O and vortexed for 10 sec; 50 μl of 2.5 M CaCl₂ is added, vortexed for 5 sec, followed by the addition of 20 μl of 0.1 M spermidine which is also vortexed for 5 sec. The gold is then allowed to settle to the bottom of the microfuge tube (5-10 min) and the supernatant fluid is removed. The gold/DNA is resuspended in 200 μl of 100% ethanol, allowed to settle and the supernatant fluid is removed. The ethanol wash is repeated and the supernatant fluid is removed. The sediment is resuspended in 120 μl of 100% ethanol and aliquots of 8 μl are added to each macrocarrier. The gold is resuspended before each aliquot is removed. The macrocarriers are placed under vacuum to ensure complete evaporation of ethanol (about 5 min).

Selection:

The bombarded tissue is cultured on embryo proliferation medium described above for 12 days prior to subculture to selection medium (embryo proliferation medium containing 55 mg/l hygromycin added to autoclaved media). The tissue is sub-cultured 5 days later and weekly for the following 9 weeks. Green colonies (putative transgenic events) are transferred to a well containing 1 ml of selection media in a 24-well multi-well plate that is maintained on a flask shaker as above. The media in multi-well dishes is replaced with fresh media every 2 weeks until the colonies are approx. 2-4 mm in diameter with proliferative embryos, at which time they are transferred to 125 ml Erlenmeyer flasks containing 30 ml of selection medium. A portion of the proembryos from transgenic events is harvested to examine gene expression by RT-PCR.

Plant Regeneration:

Maturation of embryos is carried out, without selection, at conditions described for embryo induction. Embryogenic clusters are cultured on Petri dishes containing maturation medium (MS salts, B5 vitamins, 6% maltose, 0.2% gelrite gellan gum (Sigma), 750 mg/l MgCl₂, pH 5.7) with 0.5% activated charcoal for 5-7 days and without activated charcoal for the following 3 weeks. Embryos (10-15 per event) with apical meristems are selected under a dissection microscope and cultured on a similar medium containing 0.6% phytagar (Gibco, Burlington, ON, Canada) as the solidifying agent, without the additional MgCl₂, for another 2-3 weeks or until the embryos become pale yellow in color. A portion of the embryos from transgenic events after varying times on gelrite are harvested to examine gene expression by RT-PCR.

Mature embryos are desiccated by transferring embryos from each event to empty Petri dish bottoms that are placed inside Magenta boxes (Sigma) containing several layers of sterile Whatman filter paper flooded with sterile water, for 100% relative humidity. The Magenta boxes are covered and maintained in darkness at 20° C. for 5-7 days. The embryos are germinated on solid B5 medium containing 2% sucrose, 0.2% gelrite and 0.075% MgCl₂ in Petri plates, in a chamber at 20° C., 20-h photoperiod under cool white fluorescent lights at 35-75 μmol m⁻² s⁻¹. Germinated embryos with unifoliate or trifoliate leaves are planted in artificial soil (Sunshine Mix No. 3, SunGro Horticulture Inc., Bellevue, Wash., USA), and covered with a transparent plastic lid to maintain high humidity. The flats are placed in a controlled growth cabinet at 26/24° C. (day/night), 18 h photoperiod at a light intensity of 150 μmol m⁻² s⁻¹. At the 2-3 trifoliate stage (2-3 weeks), the plantlets with strong roots are transplanted to pots containing a 3:1:1:1 mix of ASB Original Grower Mix (a peat-based mix from Greenworld, ON, Canada): soil:sand:perlite and grown at 18-h photoperiod at a light intensity of 300-400 μmol m⁻² s⁻¹.

T1 seeds are harvested and planted in soil and grown in a controlled growth cabinet at 26/24° C. (day/night), 18 h photoperiod at a light intensity of 300-400 μmol m⁻² s⁻¹. Plants are grown to maturity and T2 seed is harvested.

Plant tissue from the T1 and T2 generations are screened for edits using Next Generation Sequencing as previously described for rice.

Brassica napus:

Agrobacterium-mediated transformation of Brassica napus can be performed as follows.

In preparation for plant transformation experiments, seeds of Brassica napus cv DH12075 (obtained from Agriculture and Agri-Food Canada) are surface sterilized with sufficient 95% ethanol for 15 seconds, followed by 15 minutes incubation with occasional agitation in full strength Javex (or other commercial bleach, 7.4% sodium hypochlorite) and a drop of wetting agent such as Tween 20. The Javex solution is decanted and 0.025% mercuric chloride with a drop of Tween 20 is added and the seeds are sterilized for another 10 minutes. The seeds are then rinsed three times with sterile distilled water. The sterilized seeds are plated on half strength hormone-free Murashige and Skoog (MS) media (Murashige T, Skoog F (1962). Physiol Plant 15:473-498) with 1% sucrose in 15×60 mm petri dishes that are then placed, with the lid removed, into a larger sterile vessel (Majenta GA7 jars). The cultures are kept at 25° C., with 16 h light/8h dark, under approx. 70-80 μmol m⁻² s⁻¹ of light intensity in a tissue culture cabinet. 4-5 days old seedlings are used to excise fully unfolded cotyledons along with a small segment of the hypocotyl. Excisions are made so as to ensure that no part of the apical meristem is included.

The Agrobacterium strain GV3101 carrying the transformation vector for genome editing is grown overnight in 5 ml of LB media with 50 mg/L kanamycin, gentamycin, and rifampicin. The culture is centrifuged at 2000 g for 10 min., the supernatant is discarded and the pellet is suspended in 5 ml of inoculation medium (Murashige and Skoog with B5 vitamins [MS/B5; Gamborg O L, Miller R A, Ojima K. Exp Cell Res 50:151-158], 3% sucrose, 0.5 mg/L benzyl aminopurine (BA), pH 5.8). Cotyledons are collected in Petri dishes with ˜1 ml of sterile water to keep them from wilting. The water is removed prior to inoculation and explants are inoculated in mixture of 1 part Agrobacterium suspension and 9 parts inoculation medium in a final volume sufficient to bathe the explants. After explants are well exposed to the Agrobacterium solution and inoculated, a pipet is used to remove any extra liquid from the petri dishes.

The Petri plates containing the explants incubated in the inoculation media are sealed and kept in the dark in a tissue culture cabinet set at 25° C. After 2 days the cultures are transferred to 4° C. and incubated in the dark for 3 days. The cotyledons, in batches of 10, are then transferred to selection medium consisting of Murashige Minimal Organics (Sigma), 3% sucrose, 4.5 mg/L BA, 500 mg/L MES, 27.8 mg/L Iron (II) sulfate heptahydrate, pH 5.8, 0.7% Phytagel with 300 mg/L timentin, and 2 mg/L L-phosphinothricin (L-PPT) added after autoclaving. The cultures are kept in a tissue culture cabinet set at 25° C., 16 h/8h, with a light intensity of about 125 μmol m⁻² s⁻¹. The cotyledons are transferred to fresh selection every 3 weeks until shoots are obtained. The shoots are excised and transferred to shoot elongation media containing MS/B5 media, 2% sucrose, 0.5 mg/L BA, 0.03 mg/L gibberellic acid (GA₃), 500 mg/L 4-morpholineethanesulfonic acid (MES), 150 mg/L phloroglucinol, pH 5.8, 0.9% Phytagar and 300 mg/L timentin and 3 mg/L L-phosphinothricin added after autoclaving. After 3-4 weeks any callus that formed at the base of shoots with normal morphology is cut off and shoots are transferred to rooting media containing half strength MS/B5 media with 1% sucrose and 0.5 mg/L indole butyric acid, 500 mg/L MES, pH 5.8, 0.8% agar, with 1.5 mg/L L-PPT and 300 mg/L timentin added after autoclaving. The plantlets with healthy shoots are hardened and transferred to 6″ pots in the greenhouse to collect T1 transgenic seeds.

Plant tissue from the T1 and T2 generations are screened for edits using Next Generation Sequencing as previously described for rice.

Transformation of protoplasts of Brassica napus can be performed as follows.

Protoplast Isolation:

Seeds of Brassica napus are surface sterilized with 70% ethanol for 2 min followed by gentle shaking in 0.4% hypochlorite solution for 20 min. The seeds are washed three times in double distilled water, and sown on sterilized ½ MS media in Petri plates that are placed without the lids in sterile Majenta jars. Protoplasts are isolated from 40 newly expanding leaves of Brassica plants. The mid vein is removed and the abaxial surface of the leaves are gently scored with a sterile scalpel. The leaves are then floated with abaxial side down in Petri plates containing 15 ml of Enzyme B2 solution (B5 salts, 1% Onozuka R 10, 0.2% Macerozyme R 10, 13% sucrose, 5 mM CaCl2.2H2O, 0.5% Polyvinylpyrrolidone, 1 mg/L NAA, 1 mg/L 2, 4-D, 1 mg/L BA, MES 0.05%, pH 6.0). Petri plates are sealed with Parafilm and leaves incubated overnight at 22° C. in the dark without shaking. Following the overnight incubation the plates are gently agitated by hand and incubation continued for 15-20 min on a rotary shaker set at 20 rpm. The digested material, consisting of a crude protoplast suspension, is then filtered through a funnel lined with 63 μm nylon screen and the filtrate collected in 50 ml falcon centrifuge tubes. An equal volume of 17% B5 wash solution (B5 salts, 5 mM CaCl2.2H2O, 17% sucrose, 0.06% MES, pH 6.0) is added to the filtrate and centrifuged at 100 g for 10 minutes. The protoplast enriched fraction (˜4 ml) floating in the form of a ring is carefully removed and transferred to fresh 15 ml falcon tubes and 11 ml of WW5-2 media (0.1 M CaCl2.2H2O, 0.2 M NaCl, 4 mM KCl, 0.08% Glucose, 0.1% MES, pH 6.0) is added per tube. The resulting suspension is gently mixed by inversion and then centrifuged at 100 g for 5 minutes. After centrifugation the supernatant is carefully decanted and discarded and the pellet consisting of an enriched protoplast fraction is retained. Protoplasts are washed twice with WW5-2 solution followed by centrifugation at 100 g and resuspended in 5 ml of WW5-2 media. The density of protoplasts is counted with a hemocytometer using a small drop of the protoplast suspension. The suspension is cooled in a refrigerator (2-8° C.) for 40-45 min.

Brassica napus protoplast transfection and culture: For protoplast transfection, the protoplasts after cold incubation are pelleted by centrifugation at 100 g for 3 minutes and then resuspended in WMMM media (15 mM MgCl₂-6H₂O, 0.4 M Mannitol, 0.1 M (CaNO₃)₂, 0.1% MES, pH 6) to a density of 2×10⁶ protoplasts per ml. 500 μl of protoplast suspension is dispensed into 15 ml falcon tubes and 50 μl of a mixture consisting of 50 μg of DNA containing the genetic construct for editing is added to the protoplast suspension and mixed by shaking. 500 μl of PEGB2 (40% PEG 4000, 0.4 M Mannitol, 0.1 M Calcium Nitrate, 0.1% MES, pH 6.0) is added gently to the protoplast DNA mixture while continuously shaking the tube. The mixture is incubated for 20 min with periodic gentle shaking. Subsequently WW5-2 media is gradually added in two stages, first a 5 ml aliquot of WW5-2 is added to the protoplast mixture which is then allowed to incubate for 10 minutes followed by addition of a second 5 ml aliquot of WW5-2 solution and incubation for 10 min. After the second incubation, the protoplasts are carefully resuspended and then pelleted by centrifugation. The protoplast pellet is resuspended in 12 ml of WW5-2 solution then pelleted by centrifugation at 100 g for 5 min. The pellet is washed once more in 10 ml of WW5-2 then pelleted by centrifugation at 100 g for 3 min. The protoplast pellet is resuspended in K3P4 medium (Kao's basal salts, 6.8% Glucose, 1% MES, 0. 5% Ficoll 400, 2 mM CaCl₂.2H₂O, 1 mg/L 2, 4-D, 1 mg/L NAA, 1 mg/L Zeatin, pH 5.8, 200 mg/L Carbenicillin, 200 mg/L Cefotaxime) at a density of 1×10⁵ protoplasts per ml and 1.5 ml of the suspension is dispensed per 60×15 mm petri plate. The plates are sealed with Parafilm and maintained in plastic boxes with opaque lids at 22° C., 16 h photoperiod, under dim fluorescent lights (25 μmol m⁻² s⁻¹).

Brassica napus—Proliferation of Calli and Regeneration of Edited Lines:

After 4-5 days the protoplast cultures are fed with 1-1.25 ml of medium consisting of a 1:1 mixture of K3P4 medium and EmBed BI medium (MS Basal salts, 3.4% sucrose, 0.05% MES, 1 mg/L NAA, 1 mg/L 2,4-D and 1 mg/L BA, pH 6.0). The plates are resealed and placed under dim light for 1-2 days and then under medium light (60-80 μmol m⁻² s⁻¹). After 4-5 days, the protoplasts are fed with 4.5 ml of a 3:1 mixture of K3P4: Embed BI medium. The plate contents are then transferred to a 100×75 mm plate and 3 ml of lukewarm Embed BI medium containing 2.1% SeaPlaque agarose is added to the protoplast suspension. The contents of the plate are swirled to gently mix the protoplast suspension with the semi-solid media and the plates are allowed to solidify in the tissue culture flow hood. Plates are sealed and cultured under dim light conditions for a week. After 7-9 days, the embedded protoplast cultures in each plate are cut into 6-8 wedges and transferred onto two plates of Proliferation B1 media (MS Basal salts, 3.4% sucrose, 0.05% MES, 1 mg/L NAA, 1 mg/L 2,4-D and 1 mg/L BA, pH 6.0, 0.8% sea plaque agarose, 200 mg/L Carbenicillin, 200 mg/L Cefotaxime) with appropriate selection for the DNA insert if stable transformation for genome editing is being performed. For transient editing, no selection is required. Proliferation plates are incubated under dim light for first 1-2 days and then moved to bright light (150 μmol m⁻² s⁻¹). Green surviving colonies are obtained after 3 to 4 weeks at which point they are transferred to fresh Proliferation B 1 plates for an additional 2-3 weeks. Large green calli are transferred to Regeneration B2 Plates (MS Basal salts, 3% sucrose, 30 μM AgNO₃, 0.05% polyvinylpyrrolidone, 0.05% MES, 0.1 mg/L NAA, 5 mg/l N6-(2-isopentenyl)adenine (2-iP), 0.1 μg/L GA3, pH 5.8, 0.8% sea plaque agarose, 100 mg/L Carbenicillin, 100 mg/L Cefotaxime) with appropriate selection, if required. Calli are transferred to fresh Regeneration B2 plates every 3 to 4 weeks. Shoots with normal morphology are transferred to rooting medium (B5 salts+0.1 mg/L NAA) and incubated under dim light conditions. Plantlets are potted in a soilless mix (Sunshine Mix 4) in 6″ pots and irrigated with NPK (20-20-20) fertilizer. Plantlets are acclimatized under plastic cups for 5-6 days and maintained in a growth room at 22° C./18° C. and a 16 hour photoperiod under 200-300 μmol m⁻² s⁻¹ light. Plants are transferred to a greenhouse and grown until T1 seed set.

Plant tissue from the T1 and T2 generations are screened for edits using Next Generation Sequencing as previously described for rice.

Camelina:

In preparation for plant transformation experiments, seeds of Camelina sativa germplasm 10CS0043 (abbreviated WT43, obtained from Agriculture and Agri-Food Canada) are sown directly into 4 inch (10 cm) pots filled with soil in the greenhouse. Growth conditions are maintained at 24° C. during the day and 18° C. during the night. Plants are grown until flowering. Plants with a number of unopened flower buds are used in ‘floral dip’ transformations.

Agrobacterium strain GV3101 (pMP90) is transformed with the binary editing construct using electroporation. A single colony of GV3101 (pMP90) containing the construct of interest is obtained from a freshly streaked plate and is inoculated into 5 mL LB medium. After overnight growth at 28° C., 2 mL of culture is transferred to a 500-mL flask containing 300 mL of LB and incubated overnight at 28° C. Cells are pelleted by centrifugation (6,000 rpm, 20 min), and diluted to an OD600 of −0.8 with infiltration medium containing 5% sucrose and 0.05% (v/v) Silwet-L77 (Lehle Seeds, Round Rock, Tex., USA). Camelina plants are transformed by “floral dip” using the transformation constructs as follows. Pots containing plants at the flowering stage are placed inside a 460 mm height vacuum desiccator (Bel-Art, Pequannock, N.J., USA). Inflorescences are immersed into the Agrobacterium inoculum contained in a 500-ml beaker. A vacuum (85 kPa) is applied and held for 5 min. Plants are removed from the desiccator and are covered with plastic bags in the dark for 24 h at room temperature. Plants are removed from the bags and returned to normal growth conditions within the greenhouse for seed formation (T1 generation of seed).

Where a visual marker is used such as DsRed, a red fluorescent protein from the Discoma genus of coral (Matz et al., 1999, Nat. Biotechnol. 17, 969-973), transgenic seeds are selected based on their fluorescence as previously described (Malik et al., Plant Biotechnol. J., 2015, 13, 675-688).

Where the bar gene is used as a selectable marker on the binary construct, T1 seeds are planted in soil and transgenic plants are selected by spraying a solution of 400 mg/L of the herbicide Liberty (active ingredient 15% glufosinate-ammonium). This allows identification of transgenic plants containing the bar gene on the T-DNA in the plasmid vectors.

Plant tissue from the T1 and T2 generations are screened for edits using Next Generation Sequencing as previously described for rice.

Those skilled in the art will understand that there are many transformation methods available for each crop and each of these procedures will be useful for practicing the invention as long the procedure produces a transiently or stably transformed explant (leaf, callus, protoplast, cell suspension culture, seed, seedling, flower, immature inflorescence, inflorescence, cotyledons, cotyledons with a small segment of the hypocotyl, etc.) that is capable of forming a regenerated plant or a viable seed that contains an edit.

### Example 5. Identification of Maize Orthologs to Switchgrass dTFs

The switchgrass dTF genes were used to identify maize orthologs of each dTF as follows: the switchgrass amino acid sequence of each dTF was blasted against the maize proteome (Phytozome-Ensemlb-18). The hits were ranked in order of the alignment score and the top hit was identified as the best ortholog and the three subsequent hits were labeled as homologues (Table 8). Each maize amino acid sequence was aligned pairwise with the switchgrass sequence to determine the percent coverage and percent similarity.

Guide target sequences were designed to produce sgRNAs to edit each of the dTFs in Table 8 and these guides target sequences are shown in Table 9. The strategy for using these guide target sequences to produce sgRNAs for editing different regions of the promoter or coding sequence of the maize dTFs, or excising larger fragments from the promoter and CDS of the maize dTFs, is outlined in FIGS. 6 and 7.

TABLE 8
Maize orthologs and homologs of switchgrass downregulated
downstream transcription factor genes¹

| | | Maize Ortholog | |
|---|---|---|---|
| | | (% coverage of and % | Maize homolog 1 |
| | | identity to switchgrass | (% coverage of and % identity |
| Gene | Switchgrass Gene | gene) | to switchgrass gene) |
| dTF1 | Pavirv00029177m | GRMZM2G049378 (89%, | GRMZM2G121753 (89%, |
| | (Gene: SEQ ID NO: 1, | 78%) | 75%) |
| | Protein: SEQ ID NO: 289) | (Gene: SEQ ID NO: 35, | (Gene: SEQ ID NO: 57, |
| | | Protein: SEQ ID NO: 313) | Protein: SEQ ID NO: 335) |
| dTF2 | Pavirv00003507m | GRMZM2G158117 (75%, | GRMZM2G117497 (73%, |
| | (Gene: SEQ ID NO: 2, | 86%) | 85%) |
| | Protein: SEQ ID NO: 290) | (Gene: SEQ ID NO: 36, | (Gene: SEQ ID NO: 58, |
| | | Protein: SEQ ID NO: 314) | Protein: SEQ ID NO: 336) |
| dTF3 | AP13CTG12699 at | Zm00001d017782 (69%, | GRMZM2G114503 (64%, |
| | (Gene: SEQ ID NO: 3, | 64%) | 62%) |
| | Protein: SEQ ID NO: 291) | (Gene: SEQ ID NO: 37, | (Gene: SEQ ID NO: 59, |
| | | Protein: SEQ ID NO: 315) | Protein: SEQ ID NO: 337) |
| dTF4 | Pavirv00024770m | GRMZM2G007063 (87%, | GRMZM2G015534 (98%, |
| | (Gene: SEQ ID NO: 4, | 40%) | 50%) |
| | Protein: SEQ ID NO: 292) | (Gene: SEQ ID NO: 38, | (Gene: SEQ ID NO: 60, |
| | | Protein: SEQ ID NO: 316) | Protein: SEQ ID NO: 338) |
| dTF5 | Pavirv00012672m | GRMZM2G124540 | GRMZM2G171468 (92%, |
| | (Gene: SEQ ID NO: 5, | (100%, 74%) | 68%) |
| | Protein: SEQ ID NO: 293) | (Gene: SEQ ID NO: 39, | (Gene: SEQ ID NO: 61, |
| | | Protein: SEQ ID NO: 317) | Protein: SEQ ID NO: 339) |
| dTF6 | Pavirv00006905m | GRMZM2G021777 | AC233888.1 |
| | (Gene: SEQ ID NO: 6, | (100%, 79%) | (87%, 79%) |
| | Protein: SEQ ID NO: 294) | (Gene: SEQ ID NO: 40, | (Gene: SEQ ID NO: 62, |
| | | Protein: SEQ ID NO: 318) | Protein: SEQ ID NO: 340) |
| dTF7 | Pavirv00011545m | GRMZM2G073427 (80%, | GRMZM2G019446 (75%, |
| | (Gene: SEQ ID NO: 7, | 62%) | 36%) |
| | Protein: SEQ ID NO: 295) | (Gene: SEQ ID NO: 41, | (Gene: SEQ ID NO: 63, |
| | | Protein: SEQ ID NO: 319) | Protein: SEQ ID NO: 341) |
| dTF8 | Pavirv00039321m | GRMZM2G380377 (97%, | GRMZM2G042756 (64%, |
| | (Gene: SEQ ID NO: 8, | 64%) | 71%) |
| | Protein: SEQ ID NO: 296) | (Gene: SEQ ID NO: 42, | (Gene: SEQ ID NO: 64, |
| | | Protein: SEQ ID NO: 320) | Protein: SEQ ID NO: 342) |
| dTF9 | Pavirv00007251m | GRMZM2G351330 (87%, | GRMZM2G117164 (87%, |
| | (Gene: SEQ ID NO: 9, | 76%) | 56%) |
| | Protein: SEQ ID NO: 297) | (Gene: SEQ ID NO: 43, | (Gene: SEQ ID NO: 65, |
| | | Protein: SEQ ID NO: 321) | Protein: SEQ ID NO: 343) |
| dTF10 | AP13ITG41879_s_at | GRMZM2G105348 (97%, | GRMZM2G118047 (96%, |
| | (Gene: SEQ ID NO: 10, | 74%) | 56%) |
| | Protein: SEQ ID NO: 298) | (Gene: SEQ ID NO: 44, | (Gene: SEQ ID NO: 66, |
| | | Protein: SEQ ID NO: 322) | Protein: SEQ ID NO: 344) |
| dTF11 | Pavirv00007239m | GRMZM2G137046 (85%, | GRMZM2G140355 (82%, |
| | (Gene: SEQ ID NO: 11, | 73%) | 82%) |
| | Protein: SEQ ID NO: 299) | (Gene: SEQ ID NO: 45, | (Gene: SEQ ID NO: 67, |
| | | Protein: SEQ ID NO: 323) | Protein: SEQ ID NO: 345) |
| dTF12 | Pavirv00003464m | GRMZM2G083472 (66%, | GRMZM2G010920 (81%, |
| | (Gene: SEQ ID NO: 12, | 83%) | 62%) |
| | Protein: SEQ ID NO: 300) | (Gene: SEQ ID NO: 46, | (Gene: SEQ ID NO: 68, |
| | | Protein: SEQ ID NO: 324) | Protein: SEQ ID NO: 346) |
| dTF13 | Pavirv00006072m | GRMZM2G018984 | GRMZM2G018398 (97%, |
| | (Gene: SEQ ID NO: 13, | (100%, 66%) | 34%) |
| | Protein: SEQ ID NO: 301) | (Gene: SEQ ID NO: 47, | (Gene: SEQ ID NO: 69, |
| | | Protein: SEQ ID NO: 325) | Protein: SEQ ID NO: 347) |
| dTF14 | Pavirv00000078m | GRMZM2G150260 (98%, | GRMZM2G114503 |
| | (Gene: SEQ ID NO: 14, | 72%) | (100%, 76%) |
| | Protein: SEQ ID NO: 302) | (Gene: SEQ ID NO: 48, | (Gene: SEQ ID NO: 59, |
| | | Protein: SEQ ID NO: 326) | Protein: SEQ ID NO: 337) |
| dTF15 | Pavirv00012008m | GRMZM2G470422 (96%, | GRMZM2G075956 (76%, |
| | (Gene: SEQ ID NO: 15, | 66%) | 58%) |
| | Protein: SEQ ID NO: 303) | (Gene: SEQ ID NO: 49, | (Gene: SEQ ID NO: 70, |
| | | Protein: SEQ ID NO: 327) | Protein: SEQ ID NO: 348) |
| dTF16 | AP13CTG14279ST_s_at | GRMZM2G048582 (96%, | GRMZM2G053298 (95%, |
| | (Gene: SEQ ID NO: 16, | 86%) | 88%) |
| | Protein: SEQ ID NO: 304) | (Gene: SEQ ID NO: 50, | (Gene: SEQ ID NO: 71, |
| | | Protein: SEQ ID NO: 328) | Protein: SEQ ID NO: 349) |
| dTF17 | Pavirv00053825m | GRMZM2G010871 (98%, | GRMZM2G165972 (90%, |
| | (Gene: SEQ ID NO: 17, | 87%) | 58%) |
| | Protein: SEQ ID NO: 305) | Gene: SEQ ID NO: 51, | (Gene: SEQ ID NO: 72, |
| | | Protein: SEQ ID NO: 329) | Protein: SEQ ID NO: 350) |
| dTF18 | Pavirv00008285m | GRMZM2G405368 | AC233888.1 |
| | (Gene: SEQ ID NO: 18, | (100%, 70%) | (93%, 37%) |
| | Protein: SEQ ID NO: 306) | (Gene: SEQ ID NO: 52, | (Gene: SEQ ID NO: 62, |
| | | Protein: SEQ ID NO: 330) | Protein: SEQ ID NO: 340) |
| dTF19 | Pavirv00010659m | GRMZM2G070034 (97%, | GRMZM2G171365 (98%, |
| | (Gene: SEQ ID NO: 19, | 80%) | 59%) |
| | Protein: SEQ ID NO: 307) | (Gene: SEQ ID NO: 53, | (Gene: SEQ ID NO: 73, |
| | | Protein: SEQ ID NO: 331) | Protein: SEQ ID NO: 351) |
| dTF20 | Pavirv00067953m | GRMZM2G441325 | GRMZM2G056120 (95%, |
| | (Gene: SEQ ID NO: 20, | (100%, 69%) | 42%) |
| | Protein: SEQ ID NO: 308) | (Gene: SEQ ID NO: 54, | (Gene: SEQ ID NO: 74, |
| | | Protein: SEQ ID NO: 332) | Protein: SEQ ID NO: 352) |
| dTF21 | Pavirv00005696m | GRMZM2G042895 (93%, | GRMZM2G119823 (70%, |
| | (Gene: SEQ ID NO: 21, | 44%) | 51%) |
| | Protein: SEQ ID NO: 309) | (Gene: SEQ ID NO: 55, | (Gene: SEQ ID NO: 75, |
| | | Protein: SEQ ID NO: 333) | Protein: SEQ ID NO: 353) |
| dTF22 | Pavirv00012971m | GRMZM2G017319 (94% | GRMZM2G044902 (99%, |
| | (Gene: SEQ ID NO: 22, | 76%) | 48%) |
| | Protein: SEQ ID NO: 310) | (Gene: SEQ ID NO: 56, | (Gene: SEQ ID NO: 76, |
| | | Protein: SEQ ID NO: 334) | Protein: SEQ ID NO: 354) |
| dTF59 | Pavirv00056268m | GRMZM2G089501 | GRMZM2G009478 |
| | (Gene: SEQ ID NO: 23, | (76%, 62%) | (81%, 61%) |
| | Protein: SEQ ID NO: 311) | (Gene: SEQ ID NO: 110, | (Gene: SEQ ID NO: 112, |
| | | Protein: SEQ ID NO: 388) | Protein: SEQ ID NO: 390) |
| dTF60 | Pavirv00036358m | GRMZM2G000520 | GRMZM2G368838 |
| | (Gene: SEQ ID NO: 24, | (96%, 63%) | (100%, 77%) |
| | Protein: SEQ ID NO: 312) | (Gene: SEQ ID NO: 111, | (Gene: SEQ ID NO: 113, |
| | | Protein: SEQ ID NO: 389) | Protein: SEQ ID NO: 391) |

| | Maize homolog 2 | Maize homolog 3 |
|---|---|---|
| | (% coverage of and % identity | (% coverage of and % |
| Gene | to switchgrass gene) | identity to switchgrass gene) |
| dTF1 | GRMZM2G158117 | GRMZM2G117497 (80%, |
| | (79%, 73%) | 69%) |
| | (Gene: SEQ ID NO: 36, | (Gene: SEQ ID NO: 58, |
| | Protein: SEQ ID NO: 314) | Protein: SEQ ID NO: 336) |
| dTF2 | GRMZM2G049378 (76%, | GRMZM2G121753 (97%, |
| | 62%) | 59%) |
| | (Gene: SEQ ID NO: 35, | (Gene: SEQ ID NO: 57, |
| | Protein: SEQ ID NO: 313) | Protein: SEQ ID NO: 335) |
| dTF3 | GRMZM2G031441 (82%, | GRMZM2G451116 (87%, |
| | 51%) | 57%) |
| | (Gene: SEQ ID NO: 77, | (Gene: SEQ ID NO: 94, |
| | Protein: SEQ ID NO: 355) | Protein: SEQ ID NO: 372) |
| dTF4 | GRMZM2G016150 (98%, | GRMZM2G019446 (72%, |
| | 40%) | 45%) |
| | (Gene: SEQ ID NO: 78, | (Gene: SEQ ID NO: 63, |
| | Protein: SEQ ID NO: 356) | Protein: SEQ ID NO: 341) |
| dTF5 | GRMZM2G173882 (100%, | GRMZM2G100176 (90%, |
| | 55%) | 53%) |
| | (Gene: SEQ ID NO: 79, | (Gene: SEQ ID NO: 95, |
| | Protein: SEQ ID NO: 357) | Protein: SEQ ID NO: 373) |
| dTF6 | GRMZM2G095598 (98%, | GRMZM2G038783 (52%, |
| | 52%) | 71%) |
| | (Gene: SEQ ID NO: 80, | (Gene: SEQ ID NO: 96, |
| | Protein: SEQ ID NO: 358) | Protein: SEQ ID NO: 374) |
| dTF7 | not found | not found |
| dTF8 | GRMZM2G137341 (75%, | GRMZM2G069126 (58%, |
| | 58%) | 68%) |
| | (Gene: SEQ ID NO: 81, | (Gene: SEQ ID NO: 97, |
| | Protein: SEQ ID NO: 359) | Protein: SEQ ID NO: 375) |
| dTF9 | GRMZM2G396527 (41%, | GRMZM2G041462 (84%, |
| | 85%) | 40%) |
| | (Gene: SEQ ID NO: 82, | (Gene: SEQ ID NO: 98, |
| | Protein: SEQ ID NO: 360) | Protein: SEQ ID NO: 376) |
| dTF10 | GRMZM2G086880 (77%, | GRMZM2G089525 (73%, |
| | 55%) | 54%) |
| | (Gene: SEQ ID NO: 83, | (Gene: SEQ ID NO: 99, |
| | Protein: SEQ ID NO: 361) | Protein: SEQ ID NO: 377) |
| dTF11 | GRMZM2G171912 (73%, | GRMZM2G039828 (73%, |
| | 62%) | 59%) |
| | (Gene: SEQ ID NO: 84, | (Gene: SEQ ID NO: 100, |
| | Protein: SEQ ID NO: 362) | Protein: SEQ ID NO: 378) |
| dTF12 | GRMZM2G173943 (32%, | GRMZM2G117854 (37%, |
| | 56%) | 54%) |
| | (Gene: SEQ ID NO: 85, | (Gene: SEQ ID NO: 101, |
| | Protein: SEQ ID NO: 363) | Protein: SEQ ID NO: 379) |
| dTF13 | GRMZM2G171179 (100%, | GRMZM2G052667 (51%, |
| | 32%) | 76%) |
| | (Gene: SEQ ID NO: 86, | (Gene: SEQ ID NO: 102, |
| | Protein: SEQ ID NO: 364) | Protein: SEQ ID NO: 380) |
| dTF14 | GRMZM2G049378 (72%, | GRMZM2G451116 (61%, |
| | 59%) | 71%) |
| | (Gene: SEQ ID NO: 35, | (Gene: SEQ ID NO: 94, |
| | Protein: SEQ ID NO: 313) | Protein: SEQ ID NO: 372) |
| dTF15 | GRMZM2G129428 (95%, | GRMZM2G068710 (67%, |
| | 43%) | 49%) |
| | (Gene: SEQ ID NO: 87, | (Gene: SEQ ID NO: 103, |
| | Protein: SEQ ID NO: 365) | Protein: SEQ ID NO: 381) |
| dTF16 | GRMZM2G466549 (66%, | GRMZM2G143566 (34%, |
| | 73%) | 87%) |
| | (Gene: SEQ ID NO: 88, | (Gene: SEQ ID NO: 104, |
| | Protein: SEQ ID NO: 366) | Protein: SEQ ID NO: 382) |
| dTF17 | GRMZM2G125969 (91%, | GRMZM2G132971 (90%, |
| | 55%) | 55%) |
| | (Gene: SEQ ID NO: 89, | (Gene: SEQ ID NO: 105, |
| | Protein: SEQ ID NO: 367) | Protein: SEQ ID NO: 383) |
| dTF18 | GRMZM2G095598 (93%, | GRMZM2G021777 (41%, |
| | 36%) | 44%) |
| | (Gene: SEQ ID NO: 80, | (Gene: SEQ ID NO: 40, |
| | Protein: SEQ ID NO: 358) | Protein: SEQ ID NO: 318) |
| dTF19 | GRMZM2G026223 (96%, | GRMZM2G102161 |
| | 61%) | (60%, 40%) |
| | (Gene: SEQ ID NO: 90, | (Gene: SEQ ID NO: 106, |
| | Protein: SEQ ID NO: 368) | Protein: SEQ ID NO: 384) |
| dTF20 | GRMZM5G874163 (95%, | GRMZM2G030710 (92%, |
| | 42%) | 31%) |
| | (Gene: SEQ ID NO: 91, | (Gene: SEQ ID NO: 107, |
| | Protein: SEQ ID NO: 369) | Protein: SEQ ID NO: 385) |
| dTF21 | GRMZM2G042893 (73%, | GRMZM2G076636 (43%, |
| | 49%) | 60%) |
| | (Gene: SEQ ID NO: 92, | (Gene: SEQ ID NO: 108, |
| | Protein: SEQ ID NO: 370) | Protein: SEQ ID NO: 386) |
| dTF22 | GRMZM2G177110 (99%, | GRMZM2G073044 |
| | 47%) | (49%/61%) |
| | (Gene: SEQ ID NO: 93, | (Gene: SEQ ID NO: 109, |
| | Protein: 371) | Protein: SEQ ID NO: 387) |
| dTF59 | GRMZM2G317450 (63%, | GRMZM5G839518 (31%, |
| | 49%) | 48%) |
| | (Gene: SEQ ID NO: 114, | (Gene: SEQ ID NO: 116, |
| | Protein: SEQ ID NO: 392) | Protein: SEQ ID NO: 394) |
| dTF60 | GRMZM2G434203 (97%, | GRMZM2G047918 (39%, |
| | 63%) | 62%) |
| | (Gene: SEQ ID NO: 115, | (Gene: SEQ ID NO: 117, |
| | Protein: SEQ ID NO: 393) | Protein: SEQ ID NO: 395) |

¹Gene sequence deposited under SEQ ID contains only the coding sequence of the gene.

TABLE 9
Guide target sequences for Cas9 mediated genome editing of promoters
and/or coding sequences of transcription factor genes in maize.

| | | | Guide target #1 | | | Guide target #2 | | | Guide target #3 | | |
|---|---|---|---|---|---|---|---|---|---|---|---|
| | | | | Guide | | | Guide | | | Guide | |
| | | | | target | | | target | | | target | |
| | | Sequence | | sequence | | | sequence | | | sequence | |
| | Maize | size¹, | | (5′ to | | | (5′ to | | | (5′ to | |
| Gene | ortholog | bp | Strand² | 3′) | PAM³ | Strand | 3′) | PAM | Strand | 3′) | PAM |
| dTF1 | GRMZM2G049378 | 1450 | − | TTTGATCTCG | AGG | + | TTTCAACTGCTATT | TGG | + | GCGATAC | TGG |
| | (Protein: | (SEQ | | TTGCTGCTAT | | | AGGGTA (SEQ ID | | | ATTGATA | |
| | SEQ ID NO: | ID | | (SEQ ID | | | NO: 583) | | | CATACG | |
| | 313) | NO: | | NO: 582) | | | | | | (SEQ ID | |
| | | 118) | | | | | | | | NO: 584) | |
| dTF2 | GRMZM2G158117 | 2083 | − | TATCCTCTGT | AGG | − | TCAAACATACACTT | TGG | + | ATGAATC | AGG |
| | (Protein: SEQ | (SEQ | | TGGTGCGCTA | | | TCCAGG (SEQ ID | | | GACCTGC | |
| | ID NO: 314) | ID | | (SEQ ID | | | NO: 588) | | | CACTTA | |
| | | NO: | | NO: 587) | | | | | | (SEQ ID | |
| | | 119) | | | | | | | | NO: 589) | |
| dTF3 | Zm00001d017782 | 1392 | − | CACCTAGCTA | AGG | − | TTATTGGACCAGA | GGG | − | TTAGTGT | TGG |
| | (Protein: | (SEQ | | GGAGAGATGC | | | AAGGGTA (SEQ ID | | | TATCGTG | |
| | SEQ ID NO: | ID | | (SEQ ID | | | NO: 593) | | | ACATAC | |
| | 315) | NO: | | NO: 592) | | | | | | (SEQ ID | |
| | | 120) | | | | | | | | NO: 594) | |
| dTF4 | GRMZM2G007063 | 4472 | − | AATTTCTCGG | TGG | − | AGGGCCTTAATTG | TGG | + | AGGAGTT | AGG |
| | (Protein: | (SEQ | | CTTGGTTTGG | | | GCAAACG (SEQ ID | | | GCTCTAA | |
| | SEQ ID NO: | ID | | (SEQ ID | | | NO: 598) | | | TCGGTT | |
| | 316) | NO: | | NO: 597) | | | | | | SEQ ID | |
| | | 121) | | | | | | | | NO: 599) | |
| dTF5 | GRMZM2G124540 | 2701 | + | TTCTCTGCGC | TGG | + | GTGTGAGAAGCGG | AGG | − | GATTGGC | TGG |
| | (Protein: | (SEQ | | AAAAGATTCC | | | TAACTCG (SEQ ID | | | GTTTCGG | |
| | SEQ ID NO: | ID | | (SEQ ID | | | NO: 603) | | | GACGTC | |
| | 317) | NO: | | NO: 602) | | | | | | (SEQ ID | |
| | | 122) | | | | | | | | NO: 604) | |
| dTF6 | GRMZM2G021777 | 2206 | − | AGTCACGAGA | GGG | − | TATCTAACTTGTCA | TGG | − | AGACGGA | GGG |
| | (Protein: | (SEQ | | GAGATGCCAC | | | AGCTAT (SEQ ID | | | CGGTACG | |
| | SEQ ID NO: | ID | | (SEQ ID | | | NO: 608) | | | CCCCTG | |
| | 318) | NO: | | NO: 607) | | | | | | (SEQ ID | |
| | | 123) | | | | | | | | NO: 609) | |
| dTF7 | GRMZM2G073427 | 5651 | − | GGGAGCAGCC | TGG | − | ATATAATAGAGAT | AGG | − | AAATCAG | AGG |
| | (Protein: | (SEQ | | TGAGCAATGT | | | AATCCTA (SEQ ID | | | GACCTTA | |
| | SEQ ID NO: | ID | | (SEQ ID | | | NO: 613) | | | ACTTGA | |
| | 319) | NO: | | NO: 612) | | | | | | (SEQ ID | |
| | | 124) | | | | | | | | NO: 614) | |
| dTF8 | GRMZM2G380377 | 1767 | − | TGGTTCGATG | TGG | − | GCGCGGACTGACA | GGG | + | ACAAGCC | TGG |
| | (Protein: | (SEQ | | CGGAACGCGA | | | TGCGGCA (SEQ ID | | | GACGGAA | |
| | SEQ ID NO: | ID | | (SEQ ID | | | NO: 618) | | | AACCAG | |
| | 320) | NO: | | NO: 617) | | | | | | (SEQ ID | |
| | | 125) | | | | | | | | NO: 619) | |
| dTF9 | GRMZM2G351330 | 2018 | − | GTTCTTCCTAC | GGG | − | AGATATTCTTCGGC | GGG | + | AAGACGG | TGG |
| | (Protein: SEQ | (SEQ | | CAGCCGGCC | | | CCCGGT (SEQ ID | | | CAGACCA | |
| | ID NO: 321) | ID | | (SEQ ID | | | NO: 623) | | | CCCGAC | |
| | | NO: | | NO: 622) | | | | | | (SEQ ID | |
| | | 126) | | | | | | | | NO: 624) | |
| dTF10 | GRMZM2G105348 | 2109 | − | AATGGTGATG | AGG | + | GCGCCAGCACGAA | CGG | + | CAGGTAC | AGG |
| | (Protein: | (SEQ | | GGGCGCGGAA | | | TCGTATC (SEQ ID | | | ACTAAAC | |
| | SEQ ID NO: | ID | | (SEQ ID | | | NO: 628) | | | GCGAGA | |
| | 322) | NO: | | NO: 627) | | | | | | (SEQ ID | |
| | | 127) | | | | | | | | NO: 629) | |
| dTF11 | GRMZM2G137046 | 6138 | − | TGGGAACAAC | GGG | − | ATCCACACGGGTT | CGG | + | TTGTCGC | CGG |
| | (Protein: | (SEQ | | ACAGACCGTA | | | CGCCGTT (SEQ ID | | | GGAGCCA | |
| | SEQ ID NO: | ID | | (SEQ ID | | | NO: 633) | | | AAACCC | |
| | 323) | NO: | | NO: 632) | | | | | | (SEQ ID | |
| | | 128) | | | | | | | | NO: 634) | |
| dTF12 | GRMZM2G083472 | 3744 | − | AGCCGAGGAC | CGG | + | TGTAGGGAATTTA | TGG | − | ATTCCTT | TGG |
| | (Protein: | (SEQ | | CCCAAGAGAT | | | CCGTAAA (SEQ ID | | | CACTATT | |
| | SEQ ID NO: | ID | | (SEQ ID | | | NO: 638) | | | TAGAGC | |
| | 324) | NO: | | NO: 637) | | | | | | (SEQ ID | |
| | | 129) | | | | | | | | NO: 639) | |
| dTF13 | GRMZM2G018984 | 2636 | + | GTCATCCAGT | AGG | − | CATCTTCCCAACGC | GGG | + | TGTCGAG | AGG |
| | (Protein: | (SEQ | | AAGCTGCGCT | | | CCTGCC (SEQ ID | | | CACTAAA | |
| | SEQ ID NO: | ID | | (SEQ ID | | | NO: 643) | | | GGCAGG | |
| | 325) | NO: | | NO: 642) | | | | | | (SEQ ID | |
| | | 130) | | | | | | | | NO: 644) | |
| dTF14 | GRMZM2G150260 | 2644 | − | TCGCTCTTGAT | TGG | − | TTTATCTCCTCACC | CGG | − | GTGGTTT | AGG |
| | (Protein: | (SEQ | | CGTTGCAGC | | | TCTTCC (SEQ ID | | | ACGAGTA | |
| | SEQ ID NO: | ID | | (SEQ ID | | | NO: 648) | | | TTCGAA | |
| | 326) | NO: | | NO: 647) | | | | | | (SEQ ID | |
| | | 131) | | | | | | | | NO: 649) | |
| dTF15 | GRMZM2G470422 | 2436 | + | ACCAGCTCGA | TGG | + | ATCTCCATCATTCA | GGG | + | ATTAATT | TGG |
| | (Protein: | (SEQ | | TTAGCTCAGC | | | CCGAGA (SEQ ID | | | CTTTCCG | |
| | SEQ ID NO: | ID | | (SEQ ID | | | NO: 653) | | | CGCGTG | |
| | 327) | NO: | | NO: 652) | | | | | | (SEQ ID | |
| | | 132) | | | | | | | | NO: 654) | |
| dTF16 | GRMZM2G048582 | 5789 | + | GGAGCATCGA | GGG | − | GTTTGCAAACAGG | GGG | + | AACAGTT | TGG |
| | (Protein: | (SEQ | | GCCATTTCCG | | | AGGAACT (SEQ ID | | | GTCCCGG | |
| | SEQ ID NO: | ID | | (SEQ ID | | | NO: 658) | | | AATTTG | |
| | 328) | NO: | | NO: 657) | | | | | | (SEQ ID | |
| | | 133) | | | | | | | | NO: 659) | |
| dTF17 | GRMZM2G010871 | 3728 | + | CCGTGTTCCG | TGG | − | TTTTCTTTTTTGGC | AGG | − | GGGTCAT | TGG |
| | (Protein: | (SEQ | | TGCCACAATC | | | GCAAAC (SEQ ID | | | CAGTATT | |
| | SEQ ID NO: | ID | | (SEQ ID | | | NO: 663) | | | ATCTTT | |
| | 329) | NO: | | NO: 662) | | | | | | (SEQ ID | |
| | | 134) | | | | | | | | NO: 664) | |
| dTF18 | GRMZM2G405368 | 5305 | + | CATCGTAGTA | AGG | − | AGCTTGGTCGCAT | TGG | + | ATAAAAG | TGG |
| | (Protein: | (SEQ | | ACTGCCTCAT | | | TACGAAT (SEQ ID | | | GGTATAT | |
| | SEQ ID NO: | ID | | (SEQ ID | | | NO: 668) | | | GTTTAG | |
| | 330) | NO: | | NO: 667) | | | | | | (SEQ ID | |
| | | 135) | | | | | | | | NO: 669) | |
| dTF19 | GRMZM2G070034 | 2528 | + | ATGAGTTGCA | GGG | − | ATATATTGGTGATA | TGG | + | AAGACAT | AGG |
| | (Protein: | (SEQ | | GAATGTTTCT | | | AAGCAA (SEQ ID | | | GGAGCTC | |
| | SEQ ID NO: | ID | | (SEQ ID | | | NO: 673) | | | ATTAAG | |
| | 331) | NO: | | NO: 672) | | | | | | (SEQ ID | |
| | | 136) | | | | | | | | NO: 674) | |
| dTF20 | GRMZM2G441325 | 7046 | − | AACCAGCGAG | TGG | − | CAGCGTCACGCTC | AGG | + | GCTTGAT | GGG |
| | (Protein: | (SEQ | | GGAACGTTGA | | | CCGGGAA (SEQ ID | | | AAAGAAT | |
| | SEQ ID NO: | ID | | (SEQ ID | | | NO: 678) | | | CTCGTA | |
| | 332) | NO: | | NO: 677) | | | | | | (SEQ ID | |
| | | 137) | | | | | | | | NO: 679) | |
| dTF21 | GRMZM2G042895 | 2852 | + | TTCCCGCAGC | AGG | − | AATAGTATTGGCG | TGG | − | AATCCAT | TGG |
| | (Protein: | (SEQ | | GAGCATCAAA | | | TCTAACA (SEQ ID | | | ACAAGAG | |
| | SEQ ID NO: | ID | | (SEQ ID | | | NO: 683) | | | AGTCCA | |
| | 333) | NO: | | NO: 682) | | | | | | (SEQ ID | |
| | | 138) | | | | | | | | NO: 684) | |
| dTF22 | GRMZM2G017319 | 3237 | + | CATTAAACGT | AGG | + | TGAGCGCGCCAGG | TGG | − | GCGTGAC | CGG |
| | (Protein: | (SEQ | | ACGAGACTGC | | | TACGTGG (SEQ ID | | | GTACGCC | |
| | SEQ ID NO: | ID | | (SEQ ID | | | NO: 687) | | | GCGGAA | |
| | 334) | NO: | | NO: 555) | | | | | | (SEQ ID | |
| | | 28) | | | | | | | | NO: 688) | |
| dTF59⁴ | GRMZM2G089501 | 8185 | − | CGCAATTCTCT | TGG | + | TCGTTACCGCAAAC | CGG | + | CGGAGGG | CGG |
| | (Protein: | (SEQ | | TGGGCCGGT | | | GTTGTA (SEQ ID | | | GACGCAT | |
| | SEQ ID NO: | ID | | (SEQ ID | | | NO: 690) | | | TGCGTA | |
| | 388) | NO: | | NO: 689) | | | | | | (SEQ ID | |
| | | 139) | | | | | | | | NO: 691) | |
| dTF60 | GRMZM2G000520 | 2574 | − | GCAGACGCGT | GGG | + | TATGCTAATATCCC | TGG | − | TACGCAT | TGG |
| | (Protein: | (SEQ | | AAATCCGAGC | | | CCGTTT (SEQ ID | | | GTCTAGT | |
| | SEQ ID NO: | ID | | (SEQ ID | | | NO: 695) | | | AACGCA | |
| | 389) | NO: | | NO: 694) | | | | | | (SEQ ID | |
| | | 140) | | | | | | | | NO: 696) | |

| | | | Guide target #4 | | | Guide target #5 | | |
|---|---|---|---|---|---|---|---|---|
| | | | | Guide | | | Guide | |
| | | | | target | | | target | |
| | | Sequence | | sequence | | | sequence | |
| | Maize | size¹, | | (5′ to | | | (5′ to | |
| Gene | ortholog | bp | Strand | 3′) | PAM | Strand | 3′) | PAM |
| dTF1 | GRMZM2G049378 | 1450 | − | GTTGTGAGACGAA | TGG | − | GCTCCCT | GGG |
| | (Protein: | (SEQ | | GAGCTCC (SEQ | | | GCGTGTT | |
| | SEQ ID NO: | ID | | ID NO: 585) | | | GTAGTT | |
| | 313) | NO: | | | | | (SEQ ID | |
| | | 118) | | | | | NO: 586) | |
| dTF2 | GRMZM2G158117 | 2083 | + | CTTCAAATATGTCT | CGG | + | AGTACAA | TGG |
| | (Protein: SEQ | (SEQ | | GCCTCG (SEQ ID | | | GACACAG | |
| | ID NO: 314) | ID | | NO: 590) | | | GGATTC | |
| | | NO: | | | | | (SEQ ID | |
| | | 119) | | | | | NO: 591) | |
| dTF3 | Zm00001d017782 | 1392 | + | ACCGCTGGCACAA | CGG | + | GCAGCAG | AGG |
| | (Protein: | (SEQ | | CGTTGCG (SEQ | | | CAGTTCT | |
| | SEQ ID NO: | ID | | ID NO: 595) | | | TGGGGC | |
| | 315) | NO: | | | | | (SEQ ID | |
| | | 120) | | | | | NO: 596) | |
| dTF4 | GRMZM2G007063 | 4472 | − | ACGCGCTCCATTCC | AGG | − | AGGTCGA | GGG |
| | (Protein: | (SEQ | | GGATCG (SEQ ID | | | GCCGGAT | |
| | SEQ ID NO: | ID | | NO: 600) | | | GAAGCC | |
| | 316) | NO: | | | | | (SEQ ID | |
| | | 121) | | | | | NO: 601) | |
| dTF5 | GRMZM2G124540 | 2701 | − | CGAGGTCCAGACC | AGG | − | GCCGACG | CGG |
| | (Protein: | (SEQ | | CAGATCC (SEQ | | | GTGGCCG | |
| | SEQ ID NO: | ID | | ID NO: 605) | | | CTACAC | |
| | 317) | NO: | | | | | (SEQ ID | |
| | | 122) | | | | | NO: 606) | |
| dTF6 | GRMZM2G021777 | 2206 | + | AGAAGCCGGCGGC | TGG | + | GGGCGTG | GGG |
| | (Protein: | (SEQ | | GGGGTAC (SEQ | | | CTTCTCG | |
| | SEQ ID NO: | ID | | ID NO: 610) | | | CCCACG | |
| | 318) | NO: | | | | | (SEQ ID | |
| | | 123) | | | | | NO: 611) | |
| dTF7 | GRMZM2G073427 | 5651 | − | AGCACCGCGGCGT | GGG | − | GAATGTC | CGG |
| | (Protein: | (SEQ | | ATGCGCC (SEQ | | | GTTGACG | |
| | SEQ ID NO: | ID | | ID NO: 615) | | | CGGTCT | |
| | 319) | NO: | | | | | (SEQ ID | |
| | | 124) | | | | | NO: 616) | |
| dTF8 | GRMZM2G380377 | 1767 | + | TCGGCTACGGCTA | GGG | − | CACGATG | CGG |
| | (Protein: | (SEQ | | CGGGTAC (SEQ | | | CTATGCC | |
| | SEQ ID NO: | ID | | ID NO: 620) | | | ATCCGC | |
| | 320) | NO: | | | | | (SEQ ID | |
| | | 125) | | | | | NO: 621) | |
| dTF9 | GRMZM2G351330 | 2018 | − | CCCGCCAAAAACG | CGG | − | GGCATGG | CGG |
| | (Protein: SEQ | (SEQ | | AGGACGG (SEQ | | | CGCAGTA | |
| | ID NO: 321) | ID | | ID NO: 625) | | | GGACTC | |
| | | NO: | | | | | (SEQ ID | |
| | | 126) | | | | | NO: 626) | |
| dTF10 | GRMZM2G105348 | 2109 | + | CGTGGCCAAGACG | TGG | + | AGGACGG | AGG |
| | (Protein: | (SEQ | | TACCGCA (SEQ | | | CGCGAGC | |
| | SEQ ID NO: | ID | | ID NO: 630) | | | ATCAAG | |
| | 322) | NO: | | | | | (SEQ ID | |
| | | 127) | | | | | NO: 631) | |
| dTF11 | GRMZM2G137046 | 6138 | − | CTCCATGTCCACGT | CGG | + | GCTGTAA | AGG |
| | (Protein: | (SEQ | | GGTGCC (SEQ | | | ACAGAAG | |
| | SEQ ID NO: | ID | | ID NO: 635) | | | AGGTTC | |
| | 323) | NO: | | | | | (SEQ ID | |
| | | 128) | | | | | NO: 636) | |
| dTF12 | GRMZM2G083472 | 3744 | − | GATGCGAGACGTG | GGG | − | CACAATT | TGG |
| | (Protein: | (SEQ | | AGACTGT (SEQ | | | GTATACC | |
| | SEQ ID NO: | ID | | ID NO: 640) | | | TGCAGA | |
| | 324) | NO: | | | | | (SEQ ID | |
| | | 129) | | | | | NO: 641) | |
| dTF13 | GRMZM2G018984 | 2636 | + | GCGTCGAAGCCGG | AGG | + | CTGCAGA | TGG |
| | (Protein: | (SEQ | | TGACAGA (SEQ | | | TGCCCTA | |
| | SEQ ID NO: | ID | | ID NO: 645) | | | CTCGGG | |
| | 325) | NO: | | | | | (SEQ ID | |
| | | 130) | | | | | NO: 646) | |
| dTF14 | GRMZM2G150260 | 2644 | − | GTCCGCCGACTTG | CGG | − | TCGGCGT | GGG |
| | (Protein: | (SEQ | | CCACCCA (SEQ | | | CGTCGTA | |
| | SEQ ID NO: | ID | | ID NO: 650) | | | GCCGCC | |
| | 326) | NO: | | | | | (SEQ ID | |
| | | 131) | | | | | NO: 651) | |
| dTF15 | GRMZM2G470422 | 2436 | + | CGCGAGCCGACCC | GGG | + | GACGAGG | TGG |
| | (Protein: | (SEQ | | AAGCCGG (SEQ | | | GTCGCTC | |
| | SEQ ID NO: | ID | | ID NO: 655) | | | TCACCC | |
| | 327) | NO: | | | | | (SEQ ID | |
| | | 132) | | | | | NO: 656) | |
| dTF16 | GRMZM2G048582 | 5789 | − | GTGCGTGCCACGG | CGG | + | CCAGCTA | TGG |
| | (Protein: | (SEQ | | AGATGCT (SEQ | | | CAAGGGT | |
| | SEQ ID NO: | ID | | ID NO: 660) | | | CACAGC | |
| | 328) | NO: | | | | | (SEQ ID | |
| | | 133) | | | | | NO: 661) | |
| dTF17 | GRMZM2G010871 | 3728 | − | CCCACACCGTCCTG | CGG | + | ACACACC | GGG |
| | (Protein: | (SEQ | | CGTCGG (SEQ | | | ATTCTAC | |
| | SEQ ID NO: | ID | | ID NO: 665) | | | AGTGAT | |
| | 329) | NO: | | | | | (SEQ ID | |
| | | 134) | | | | | NO: 666) | |
| dTF18 | GRMZM2G405368 | 5305 | − | AATTGCTGATGAT | TGG | + | CGCGAAA | TGG |
| | (Protein: | (SEQ | | ACTAGTC (SEQ | | | AGAGTCT | |
| | SEQ ID NO: | ID | | ID NO: 670) | | | CTGATA | |
| | 330) | NO: | | | | | (SEQ ID | |
| | | 135) | | | | | NO: 671) | |
| dTF19 | GRMZM2G070034 | 2528 | + | CATTGAAGAACTG | TGG | + | GTACATA | GGG |
| | (Protein: | (SEQ | | CATAATC (SEQ | | | GGATTAC | |
| | SEQ ID NO: | ID | | ID NO: 675) | | | CCGGCA | |
| | 331) | NO: | | | | | (SEQ ID | |
| | | 136) | | | | | NO: 676) | |
| dTF20 | GRMZM2G441325 | 7046 | + | GCGCGGTGTGCGC | TGG | + | GAAAGAA | TGG |
| | (Protein: | (SEQ | | GGAGCTG (SEQ | | | GGGCAAT | |
| | SEQ ID NO: | ID | | ID NO: 680) | | | ACGAAG | |
| | 332) | NO: | | | | | (SEQ ID | |
| | | 137) | | | | | NO: 681) | |
| dTF21 | GRMZM2G042895 | 2852 | − | GCTGGAGTCGTAG | GGG | − | TGATCTG | TGG |
| | (Protein: | (SEQ | | CAGGACA (SEQ | | | GATACGA | |
| | SEQ ID NO: | ID | | ID NO: 685) | | | TTCGTC | |
| | 333) | NO: | | | | | (SEQ ID | |
| | | 138) | | | | | NO: 686) | |
| dTF22 | GRMZM2G017319 | 3237 | − | CGTGGATCCAGTC | GGG | + | GAGGACC | CGG |
| | (Protein: | (SEQ | | GATGCTG (SEQ | | | AGACCGG | |
| | SEQ ID NO: | ID | | ID NO: 576) | | | TGATCA | |
| | 334) | NO: | | | | | (SEQ ID | |
| | | 28) | | | | | NO: 577) | |
| dTF59⁴ | GRMZM2G089501 | 8185 | − | GCGCCTTGGAGTC | AGG | + | CGGCATC | GGG |
| | (Protein: | (SEQ | | GTGTAGC (SEQ | | | TAGCAAC | |
| | SEQ ID NO: | ID | | ID NO: 692) | | | GAATTA | |
| | 388) | NO: | | | | | (SEQ ID | |
| | | 139) | | | | | NO: 693) | |
| dTF60 | GRMZM2G000520 | 2574 | − | CTGAAGCCGAACC | CGG | − | ACGGGTC | AGG |
| | (Protein: | (SEQ | | AGCCTGG (SEQ | | | ATGGTAG | |
| | SEQ ID NO: | ID | | ID NO: 697) | | | CTCCAG | |
| | 389) | NO: | | | | | (SEQ ID | |
| | | 140) | | | | | NO: 698) | |

Five guide target sequences were designed for each gene target as described in FIG. 7.
¹DNA sequence includes the 5′ UTR region upstream of the ATG (predicted at MaizeGDB (Andorf et al., 2016, Nucleic Acids Res., doi: 10.1093/nar/gkv1007), GenBank, and/or transcript analysis), 1000 bp of promoter sequence upstream of the 5′ UTR, and the coding sequence of the gene including any introns.
²Strand (+/−) refers to the gRNA binding to either the forward strand of DNA (+) or its reverse complement (−).
³PAM refers to the protospacer adjacent motif for the CAS9 enzyme that resides directly adjacent to the 3′ end of the guide RNA target.
⁴For dTF59, the gene has multiple Exons and the first Exon of the gene typically used for design of Guide target sequence #4 (FIG. 7) is very short. Thus the second exon was used to design guide target sequence #4 for dTF59.

### Example 6. Identification of Orthologs of Other dTFs in Major Crops

Orthologs of each switchgrass dTF gene were found in major crops by reciprocal BLAST searches as described above. The hits were ranked in order of the alignment score and the top hit was identified as the best ortholog. The orthologs for corn, soybean, canola, rice, Medicago truncatula (a close relative of alfalfa), sorghum, and wheat are shown in Tables 8 and 10-14. The coding sequence of the camelina ortholog of dTF22 is shown in Table 15. The genome sequence for alfalfa (Medicago sativa) is not publically available. The sequence of a close relative to alfalfa, Medicago truncatula was used to find the dTF22 ortholog. Once the genome sequence of alfalfa (Medicago sativa) is publically available, the dTF22 ortholog can be found by comparison with the Medicago truncatula and switchgrass genes.

Guide target sequences to produce sgRNAs (FIG. 6) can be designed for CRISPR/Cas9 editing of each gene or promoter sequence as described above and as detailed in FIG. 7. The Gene IDs listed in Tables 10-15 can be used to download the sequence of the promoter in front of each gene, using a database such as Phytozome, if the promoter sequence is the target for editing. When designing sgRNAs for editing, the genomic sequence for each gene can be downloaded to locate any introns that might affect the choice of design of sgRNAs.

Editing of the dTF can be achieved through Agrobacterium mediated transformation, protoplast transformation, transformation of explants with the gene gun, or delivery or through the use of RNPs as previously described above.

TABLE 10
Orthologs of dTFs in corn¹

| | |
|---|---|
| Switchgrass | Corn |

| dTF | Gene | SEQ ID |
|---|---|---|
| 1 | GRMZM2G049378 | Gene: SEQ ID NO: 35 |
| | | Protein: SEQ ID NO: 313 |
| 2 | GRMZM2G158117 | Gene: SEQ ID NO: 36 |
| | | Protein: SEQ ID NO: 314 |
| 3 | Zm00001d017782 | Gene: SEQ ID NO: 37 |
| | | Protein: SEQ ID NO: 315 |
| 4 | GRMZM2G007063 | Gene: SEQ ID NO: 38 |
| | | Protein: SEQ ID NO: 316 |
| 5 | GRMZM2G124540 | Gene: SEQ ID NO: 39 |
| | | Protein: SEQ ID NO: 317 |
| 6 | GRMZM2G021777 | Gene: SEQ ID NO: 40 |
| | | Protein: SEQ ID NO: 318 |
| 7 | GRMZM2G073427 | Gene: SEQ ID NO: 41 |
| | | Protein: SEQ ID NO: 319 |
| 8 | GRMZM2G380377 | Gene: SEQ ID NO: 42 |
| | | Protein: SEQ ID NO: 320 |
| 9 | GRMZM2G351330 | Gene: SEQ ID NO: 43 |
| | | Protein: SEQ ID NO: 321 |
| 10 | GRMZM2G105348 | Gene: SEQ ID NO: 44 |
| | | Protein: SEQ ID NO: 322 |
| 11 | GRMZM2G137046 | Gene: SEQ ID NO: 45 |
| | | Protein: SEQ ID NO: 323 |
| 12 | GRMZM2G083472 | Gene: SEQ ID NO: 46 |
| | | Protein: SEQ ID NO: 324 |
| 13 | GRMZM2G018984 | Gene: SEQ ID NO: 47 |
| | | Protein: SEQ ID NO: 325 |
| 14 | GRMZM2G150260 | Gene: SEQ ID NO: 48 |
| | | Protein: SEQ ID NO: 326 |
| 15 | GRMZM2G470422 | Gene: SEQ ID NO: 49 |
| | | Protein: SEQ ID NO: 327 |
| 16 | GRMZM2G048582 | Gene: SEQ ID NO: 50 |
| | | Protein: SEQ ID NO: 328 |
| 17 | GRMZM2G010871 | Gene: SEQ ID NO: 51 |
| | | Protein: SEQ ID NO: 329 |
| 18 | GRMZM2G405368 | Gene: SEQ ID NO: 52 |
| | | Protein: SEQ ID NO: 330 |
| 19 | GRMZM2G070034 | Gene: SEQ ID NO: 53 |
| | | Protein: SEQ ID NO: 331 |
| 20 | GRMZM2G441325 | Gene: SEQ ID NO: 54 |
| | | Protein: SEQ ID NO: 332 |
| 21 | GRMZM2G042895 | Gene: SEQ ID NO: 55 |
| | | Protein: SEQ ID NO: 333 |
| 22 | GRMZM2G017319 | Gene: SEQ ID NO: 56 |
| | | Protein: SEQ ID NO: 334 |
| 59 | GRMZM2G089501 | Gene: SEQ ID NO: 110 |
| | | Protein: SEQ ID NO: 388 |
| 60 | GRMZM2G000520 | Gene: SEQ ID NO: 111 |
| | | Protein: SEQ ID NO: 389 |

Sequence deposited under SEQ ID contains only the coding sequence of the gene.

TABLE 11
Soybean orthologs and homologs of switchgrass downregulated downstream transcription factor genes¹

| Switchgrass | Ortholog | | Homolog | |
|---|---|---|---|---|
| dTF | Gene | SEQ ID | Gene | SEQ ID |
| 1 | Glyma.12G042900 | Gene: SEQ ID NO: 141 | Glyma.11G117200.1 | Gene: SEQ ID NO: 142 |
| | | Protein: SEQ ID NO: 396 | | Protein: SEQ ID NO: 397 |
| 2 | Glyma.11G117200 | Gene: SEQ ID NO: 142 | Glyma.12G042900.1 | Gene: SEQ ID NO: 141 |
| | | Protein: SEQ ID NO: 397 | | Protein: SEQ ID NO: 396 |
| 3 | Glyma.14G074500 | Gene: SEQ ID NO: 143 | Glyma.11G069100.1 | Gene: SEQ ID NO: 722 |
| | | Protein: SEQ ID NO: 398 | | Protein: SEQ ID NO: 742 |
| 4 | Glyma.10G162100 | Gene: SEQ ID NO: 144 | Glyma.03G247100.1 | Gene: SEQ ID NO: 723 |
| | | Protein: SEQ ID NO: 399 | | Protein: SEQ ID NO: 743 |
| 5 | Glyma.11G058600 | Gene: SEQ ID NO: 145 | Glyma.01G183700.1 | Gene: SEQ ID NO: 724 |
| | | Protein: SEQ ID NO: 400 | | Protein: SEQ ID NO: 744 |
| 6 | Glyma.13G093800 | Gene: SEQ ID NO: 146 | Glyma.17G066600.1 | Gene: SEQ ID NO: 725 |
| | | Protein: SEQ ID NO: 401 | | Protein: SEQ ID NO: 745 |
| 7 | Glyma.10G162100 | Gene: SEQ ID NO: 144 | Glyma.20G224500.1 | Gene: SEQ ID NO: 726 |
| | | Protein: SEQ ID NO: 399 | | Protein: SEQ ID NO: 746 |
| 8 | Glyma.10G239400 | Gene: SEQ ID NO: 148 | Glyma.20G155100.1 | Gene: SEQ ID NO: 727 |
| | | Protein: SEQ ID NO: 403 | | Protein: SEQ ID NO: 747 |
| 9 | Glyma.16G021000 | Gene: SEQ ID NO: 149 | Glyma.07G052100.1 | Gene: SEQ ID NO: 728 |
| | | Protein: SEQ ID NO: 404 | | Protein: SEQ ID NO: 748 |
| 10 | Glyma.09G190600 | Gene: SEQ ID NO: 150 | Glyma.16G091800.1 | Gene: SEQ ID NO: 729 |
| | | Protein: SEQ ID NO: 405 | | Protein: SEQ ID NO: 749 |
| 11 | Glyma.18G117100 | Gene: SEQ ID NO: 151 | Glyma.08G302500.1 | Gene: SEQ ID NO: 730 |
| | | Protein: SEQ ID NO: 406 | | Protein: SEQ ID NO: 750 |
| 12 | Glyma.15G123100 | Gene: SEQ ID NO: 152 | Glyma.03G166400.1 | Gene: SEQ ID NO: 731 |
| | | Protein: SEQ ID NO: 407 | | Protein: SEQ ID NO: 751 |
| 13 | Glyma.03G263700 | Gene: SEQ ID NO: 153 | Glyma.16G012600.4 | Gene: SEQ ID NO: 732 |
| | | Protein: SEQ ID NO: 408 | | Protein: SEQ ID |
| 14 | Glyma.12G042900 | Gene: SEQ ID NO: 141 | Glyma.11G117200.1 | Gene: SEQ ID NO: 142 |
| | | Protein: SEQ ID NO: 396 | | Protein: SEQ ID NO: 397 |
| | | | Glyma.04G012600.1 | Gene: SEQ ID NO: 733 |
| | | | | Protein: SEQ ID NO: 753 |
| 15 | Glyma.10G215200 | Gene: SEQ ID NO: 155 | Glyma.20G176500.1 | Gene: SEQ ID NO: 734 |
| | | Protein: SEQ ID NO: 410 | | Protein: SEQ ID NO: 754 |
| 16 | Glyma.06G017800 | Gene: SEQ ID NO: 156 | Glyma.04G017400.9 | Gene: SEQ ID NO: 735 |
| | | Protein: SEQ ID NO: 411 | | Protein: SEQ ID NO: 755 |
| 17 | Glyma.19G159500 | Gene: SEQ ID NO: 157 | Glyma.03G157300.2 | Gene: SEQ ID NO: 736 |
| | | Protein: SEQ ID NO: 412 | | Protein: SEQ ID NO: 756 |
| 18 | Glyma.08G255200 | Gene: SEQ ID NO: 158 | Glyma.18G278100.1 | Gene: SEQ ID NO: 737 |
| | | Protein: SEQ ID NO: 413 | | Protein: SEQ ID NO: 757 |
| 19 | Glyma.09G266200 | Gene: SEQ ID NO: 159 | Glyma.18G224500.2 | Gene: SEQ ID NO: 738 |
| | | Protein: SEQ ID NO: 414 | | Protein: SEQ ID NO: 758 |
| 20 | Glyma.15G078800 | Gene: SEQ ID NO: 160 | Glyma.13G234200.1 | Gene: SEQ ID NO: 739 |
| | | Protein: SEQ ID NO: 415 | | Protein: SEQ ID NO: 759 |
| 21 | Glyma.09G060200 | Gene: SEQ ID NO: 161 | Glyma.17G058600.1 | Gene: SEQ ID NO: 740 |
| | | Protein: SEQ ID NO: 416 | | Protein: SEQ ID NO: 760 |
| 22 | Glyma05g22120 | Gene: SEQ ID NO: 162 | Glyma.05G103300.1 | Gene: SEQ ID NO: 741 |
| | | Protein: SEQ ID NO: 417 | | Protein: SEQ ID NO: 761 |
| 59 | Glyma.02G123400 | Gene: SEQ ID NO: 163 | Glyma.01G066600.1 | Gene: SEQ ID NO: 147 |
| | | Protein: SEQ ID NO: 418 | | Protein: SEQ ID NO: 402 |
| 60 | Glyma.19G192400 | Gene: SEQ ID NO: 164 | Glyma.05G049800.1 | Gene: SEQ ID NO: 154 |
| | | Protein: 419 | | Protein: SEQ ID NO: 409 |

Sequence deposited under SEQ ID contains only the coding sequence of the gene.

TABLE 12
Orthologs of dTFs in canola and rice¹

| | |
|---|---|
| Switchgrass | Canola |

| dTF | Gene | SEQ ID |
|---|---|---|
| 1 | BnaC03g76670D | Gene: SEQ ID NO: 165 |
| | | Protein: SEQ ID NO: 420 |
| 2 | BnaC01g02200D | Gene: SEQ ID NO: 166 |
| | | Protein: SEQ ID NO: 421 |
| 3 | BnaA06g13720D | Gene: SEQ ID NO: 167 |
| | | Protein: SEQ ID NO: 422 |
| 4 | BnaA02g17180D | Gene: SEQ ID NO: 168 |
| | | Protein: SEQ ID NO: 423 |
| 5 | BnaC07g11890D | Gene: SEQ ID NO: 169 |
| | | Protein: SEQ ID NO: 424 |
| 6 | BnaA04g14640D | Gene: SEQ ID NO: 170 |
| | | Protein: SEQ ID NO: 425 |
| 7 | BnaA06g29270D | Gene: SEQ ID NO: 171 |
| | | Protein: SEQ ID NO: 426 |
| 8 | BnaC04g08850D | Gene: SEQ ID NO: 172 |
| | | Protein: SEQ ID NO: 427 |
| 9 | BnaA07g24100D | Gene: SEQ ID NO: 173 |
| | | Protein: SEQ ID NO: 428 |
| 10 | BnaC03g43990D | Gene: SEQ ID NO: 174 |
| | | Protein: SEQ ID NO: 429 |
| 11 | BnaCnng20200D | Gene: SEQ ID NO: 175 |
| | | Protein: SEQ ID NO: 430 |
| 12 | BnaA01g30110D | Gene: SEQ ID NO: 176 |
| | | Protein: SEQ ID NO: 431 |
| 13 | BnaA05g23130D | Gene: SEQ ID NO: 177 |
| | | Protein: SEQ ID NO: 432 |
| 14 | BnaC06g36010D | Gene: SEQ ID NO: 178 |
| | | Protein: SEQ ID NO: 433 |
| 15 | BnaC06g08250D | Gene: SEQ ID NO: 179 |
| | | Protein: SEQ ID NO: 434 |
| 16 | BnaC08g19370D | Gene: SEQ ID NO: 180 |
| | | Protein: SEQ ID NO: 435 |
| 17 | BnaA02g03270D | Gene: SEQ ID NO: 181 |
| | | Protein: SEQ ID NO: 436 |
| 18 | BnaA10g18420D | Gene: SEQ ID NO: 182 |
| | | Protein: SEQ ID NO: 437 |
| 19 | BnaA04g26320D | Gene: SEQ ID NO: 183 |
| | | Protein: SEQ ID NO: 438 |
| 20 | BnaA03g46370D | Gene: SEQ ID NO: 184 |
| | | Protein: SEQ ID NO: 439 |
| 21 | BnaA10g11180D | Gene: SEQ ID NO: 185 |
| | | Protein: SEQ ID NO: 440 |
| 22 | BnaC02g16720D | Gene: SEQ ID NO: 186 |
| | | Protein: SEQ ID NO: 441 |
| 59 | BnaA10g23230D | Gene: SEQ ID NO: 187 |
| | | Protein: SEQ ID NO: 442 |
| 60 | BnaC09g28200D | Gene: SEQ ID NO: 188 |
| | | Protein: SEQ ID NO: 443 |

| Switchgrass | Rice | |
|---|---|---|
| dTF | Gene | SEQ ID |
| 1 | LOC_Os01g47370.1 | Gene: SEQ ID NO: 189 |
| | | Protein: SEQ ID NO: 444 |
| 2 | LOC_Os05g49240.1 | Gene: SEQ ID NO: 190 |
| | | Protein: SEQ ID NO: 445 |
| 3 | LOC_Os02g47744.4 | Gene: SEQ ID NO: 191 |
| | | Protein: SEQ ID NO: 446 |
| 4 | LOC_Os03g58250.1 | Gene: SEQ ID NO: 192 |
| | | Protein: SEQ ID NO: 447 |
| 5 | LOC_Os03g55590.1 | Gene: SEQ ID NO: 193 |
| | | Protein: SEQ ID NO: 448 |
| 6 | LOC_Os02g39710.1 | Gene: SEQ ID NO: 194 |
| | | Protein: SEQ ID NO: 449 |
| 7 | LOC_Os12g40920.1 | Gene: SEQ ID NO: 195 |
| | | Protein: SEQ ID NO: 450 |
| 8 | LOC_Os06g03670.1 | Gene: SEQ ID NO: 196 |
| | | Protein: SEQ ID NO: 451 |
| 9 | LOC_Os04g45810.1 | Gene: SEQ ID NO: 197 |
| | | Protein: SEQ ID NO: 452 |
| 10 | LOC_Os02g13800.1 | Gene: SEQ ID NO: 198 |
| | | Protein: SEQ ID NO: 453 |
| 11 | LOC_Os02g10860.1 | Gene: SEQ ID NO: 199 |
| | | Protein: SEQ ID NO: 454 |
| 12 | LOC_Os06g49040.1 | Gene: SEQ ID NO: 200 |
| | | Protein: SEQ ID NO: 455 |
| 13 | LOC_Os03g08470.3 | Gene: SEQ ID NO: 201 |
| | | Protein: SEQ ID NO: 456 |
| 14 | LOC_Os05g50340.1 | Gene: SEQ ID NO: 202 |
| | | Protein: SEQ ID NO: 457 |
| 15 | LOC_Os03g62230.1 | Gene: SEQ ID NO: 203 |
| | | Protein: SEQ ID NO: 458 |
| 16 | LOC_Os09g37710.2 | Gene: SEQ ID NO: 204 |
| | | Protein: SEQ ID NO: 459 |
| 17 | LOC_Os10g28340.5 | Gene: SEQ ID NO: 205 |
| | | Protein: SEQ ID NO: 460 |
| 18 | LOC_Os06g16370.1 | Gene: SEQ ID NO: 206 |
| | | Protein: SEQ ID NO: 461 |
| 19 | LOC_Os10g39130.1 | Gene: SEQ ID NO: 207 |
| | | Protein: SEQ ID NO: 462 |
| 20 | LOC_Os05g43920.1 | Gene: SEQ ID NO: 208 |
| | | Protein: SEQ ID NO: 463 |
| 21 | LOC_Os04g23550.1 | Gene: SEQ ID NO: 209 |
| | | Protein: SEQ ID NO: 464 |
| 22 | LOC_Os03g41330.1 | Gene: SEQ ID NO: 210 |
| | | Protein: SEQ ID NO: 465 |
| 59 | LOC_Os08g38210.1 | Gene: SEQ ID NO: 211 |
| | | Protein: SEQ ID NO: 466 |
| 60 | LOC_Os02g45420.1 | Gene: SEQ ID NO: 212 |
| | | Protein: SEQ ID NO: 467 |

Sequence deposited under SEQ ID contains only the coding sequence of the gene.

TABLE 13
Orthologs of dTFs in Medicago truncatula and sorghum¹

| Switchgrass | Medicago truncatula² | | Sorghum | |
|---|---|---|---|---|
| dTF | Gene | SEQ ID | Gene | SEQ ID |
| 1 | Medtr3g116720 | Gene: SEQ ID NO: 213 | Sb03g030330 | Gene: SEQ ID NO: 237 |
| | | Protein: SEQ ID NO: 468 | | Protein: SEQ ID NO: 492 |
| 2 | Medtr3g111880 | Gene: SEQ ID NO: 214 | Sb09g028790 | Gene: SEQ ID NO: 238 |
| | | Protein: SEQ ID NO: 469 | | Protein: SEQ ID NO: 493 |
| 3 | Medtr1g022290 | Gene: SEQ ID NO: 215 | Sb04g030510 | Gene: SEQ ID NO: 239 |
| | | Protein: SEQ ID NO: 470 | | Protein: SEQ ID NO: 494 |
| 4 | Medtr1g080920 | Gene: SEQ ID NO: 216 | Sb02g004610 | Gene: SEQ ID NO: 240 |
| | | Protein: SEQ ID NO: 471 | | Protein: SEQ ID NO: 495 |
| 5 | Medtr4g086835 | Gene: SEQ ID NO: 217 | Sb01g007130 | Gene: SEQ ID NO: 241 |
| | | Protein: SEQ ID NO: 472 | | Protein: SEQ ID NO: 496 |
| 6 | Medtr3g105710 | Gene: SEQ ID NO: 218 | Sb04g025660 | Gene: SEQ ID NO: 242 |
| | | Protein: SEQ ID NO: 473 | | Protein: SEQ ID NO: 497 |
| 7 | Medtr1g080920 | Gene: SEQ ID NO: 216 | Sb08g020600 | Gene: SEQ ID NO: 243 |
| | | Protein: SEQ ID NO: 471 | | Protein: SEQ ID NO: 498 |
| 8 | Medtr6g465530 | Gene: SEQ ID NO: 220 | Sb10g001620 | Gene: SEQ ID NO: 244 |
| | | Protein: SEQ ID NO: 475 | | Protein: SEQ ID NO: 499 |
| 9 | Medtr8g026960 | Gene: SEQ ID NO: 221 | Sb06g024000 | Gene: SEQ ID NO: 245 |
| | | Protein: SEQ ID NO: 476 | | Protein: SEQ ID NO: 500 |
| 10 | Medtr6g086805 | Gene: SEQ ID NO: 222 | Sb04g008300 | Gene: SEQ ID NO: 246 |
| | | Protein: SEQ ID NO: 477 | | Protein: SEQ ID NO: 501 |
| 11 | Medtr3g436010 | Gene: SEQ ID NO: 223 | Sb04g007060 | Gene: SEQ ID NO: 247 |
| | | Protein: SEQ ID NO: 478 | | Protein: SEQ ID NO: 502 |
| 12 | Medtr7g088070 | Gene: SEQ ID NO: 224 | Sb10g029200 | Gene: SEQ ID NO: 248 |
| | | Protein: SEQ ID NO: 479 | | Protein: SEQ ID NO: 503 |
| 13 | Medtr8g022820 | Gene: SEQ ID NO: 225 | Sb01g045060 | Gene: SEQ ID NO: 249 |
| | | Protein: SEQ ID NO: 480 | | |
| | Medtr2g105380.1 | Gene: SEQ ID NO: 762 | | Protein: SEQ ID NO: 504 |
| | | Protein: SEQ ID NO: 763 | | |
| 14 | Medtr1g022290 | Gene: SEQ ID NO: 215 | Sb03g028960 | Gene: SEQ ID NO: 250 |
| | | Protein: SEQ ID NO: 470 | | Protein: SEQ ID NO: 505 |
| 15 | Medtr1g093095 | Gene: SEQ ID NO: 227 | Sb03g041170 | Gene: SEQ ID NO: 251 |
| | | Protein: SEQ ID NO: 482 | | Protein: SEQ ID NO: 506 |
| 16 | Medtr3g115400 | Gene: SEQ ID NO: 228 | Sb02g031970 | Gene: SEQ ID NO: 252 |
| | | Protein: SEQ ID NO: 483 | | Protein: SEQ ID NO: 507 |
| 17 | Medtr7g095680 | Gene: SEQ ID NO: 229 | Sb01g021490 | Gene: SEQ ID NO: 253 |
| | | Protein: SEQ ID NO: 484 | | Protein: SEQ ID NO: 508 |
| 18 | Medtr7g018170 | Gene: SEQ ID NO: 230 | Sb10g100050 | Gene: SEQ ID NO: 254 |
| | | Protein: SEQ ID NO: 485 | | Protein: SEQ ID NO: 509 |
| 19 | Medtr8g033220 | Gene: SEQ ID NO: 231 | Sb01g030570 | Gene: SEQ ID NO: 255 |
| | | Protein: SEQ ID NO: 486 | | Protein: SEQ ID NO: 510 |
| 20 | Medtr2g014770 | Gene: SEQ ID NO: 232 | Sb09g025500 | Gene: SEQ ID NO: 256 |
| | | Protein: SEQ ID NO: 487 | | Protein: SEQ ID NO: 511 |
| 21 | Medtr2g038040 | Gene: SEQ ID NO: 233 | Sb04g017390 | Gene: SEQ ID NO: 257 |
| | | Protein: SEQ ID NO: 488 | | Protein: SEQ ID NO: 512 |
| 22 | Medtr1g106420 | Gene: SEQ ID NO: 234 | Sb01g014800 | Gene: SEQ ID NO: 258 |
| | | Protein: SEQ ID NO: 489 | | Protein: SEQ ID NO: 513 |
| 59 | Medtr4g110040 | Gene: SEQ ID NO: 235 | Sb07g028860 | Gene: SEQ ID NO: 259 |
| | | Protein: SEQ ID NO: 490 | | Protein: SEQ ID NO: 514 |
| 60 | Medtr4g102670 | Gene: SEQ ID NO: 236 | Sb06g025890 | Gene: SEQ ID NO: 260 |
| | | Protein: SEQ ID NO: 491 | | Protein: SEQ ID NO: 515 |

Sequence deposited under SEQ ID contains only the coding sequence of the gene.
²The genome sequence for alfalfa (Medicago sativa) is not publically available. The sequence of a close relative to alfalfa, Medicago truncatula was used to find the dTF22 ortholog. Once the genome sequence of alfalfa (Medicago sativa) is publically available, the dTF22 ortholog can be found by comparison with the Medicago truncatula and switchgrass genes.

TABLE 14
Orthologs of dTFs in wheat

| Switchgrass | Wheat | |
|---|---|---|
| dTF | Gene | SEQ ID |
| 1 | Traes_5BL_138F3DBE5.1 | Gene: SEQ ID NO: 261 |
| | | Protein: SEQ ID NO: 516 |
| 2 | Traes_1BL_B30884E0E.1 | Gene: SEQ ID NO: 262 |
| | | Protein: SEQ ID NO: 517 |
| 3 | Traes_6DL_3BC4F011C.1 | Gene: SEQ ID NO: 263 |
| | | Protein: SEQ ID NO: 518 |
| 4 | Traes_5AL04D3E97F0.1 | Gene: SEQ ID NO: 264 |
| | | Protein: SEQ ID NO: 519 |
| 5 | Traes_5BL_1AE458202.1 | Gene: SEQ ID NO: 265 |
| | | Protein: SEQ ID NO: 520 |
| 6 | Traes_2BL_98439EA10.1 | Gene: SEQ ID NO: 266 |
| | | Protein: SEQ ID NO: 521 |
| 7 | Traes_5AS_2F996234C.5 | Gene: SEQ ID NO: 267 |
| | | Protein: SEQ ID NO: 522 |
| 8 | Traes_5BL_0C3609EF0.2 | Gene: SEQ ID NO: 268 |
| | | Protein: SEQ ID NO: 523 |
| 9 | Traes_2BL_B69300543.1 | Gene: SEQ ID NO: 269 |
| | | Protein: SEQ ID NO: 524 |
| 10 | Traes_4BL_B64C157DC. 1 | Gene: SEQ ID NO: 270 |
| | | Protein: SEQ ID NO: 525 |
| 11 | Traes_7DL_68B814464.1 | Gene: SEQ ID NO: 271 |
| | | Protein: SEQ ID NO: 526 |
| 12 | Traes_7AL_D45376F32.2 | Gene: SEQ ID NO: 272 |
| | | Protein: SEQ ID NO: 527 |
| 13 | Traes_4AS_094442636.5 | Gene: SEQ ID NO: 273 |
| | | Protein: SEQ ID NO: 528 |
| 14 | Traes_1BL_546DFB91B.1 | Gene: SEQ ID NO: 274 |
| | | Protein: SEQ ID NO: 529 |
| 15 | Traes_5BL_BA26B A910.1 | Gene: SEQ ID NO: 275 |
| | | Protein: SEQ ID NO: 530 |
| 16 | Traes_5BL_462E6AA25.2 | Gene: SEQ ID NO: 276 |
| | | Protein: SEQ ID NO: 531 |
| 17 | Traes_1AL_A4B5C1474.1 | Gene: SEQ ID NO: 277 |
| | | Protein: SEQ ID NO: 532 |
| 18 | Traes_7AS_F46AC277B.1 | Gene: SEQ ID NO: 278 |
| | | Protein: SEQ ID NO: 533 |
| 19 | Traes_1DL_2997D073B.1 | Gene: SEQ ID NO: 279 |
| | | Protein: SEQ ID NO: 534 |
| 20 | Traes_1ALB14FE48AF.3 | Gene: SEQ ID NO: 280 |
| | | Protein: SEQ ID NO: 535 |
| 21 | Traes_2DL_DE3909A32.1 | Gene: SEQ ID NO: 281 |
| | | Protein: SEQ ID NO: 536 |
| 22 | Traes_4AL_7241716B6.1 | Gene: SEQ ID NO: 282 |
| | | Protein: SEQ ID NO: 537 |
| | Traes_4DS_9575221BB.1 | Gene: SEQ ID NO: 287 |
| | (wheat homolog to SEO ID | Protein: SEQ ID NO: 540 |
| | NO: 282) | |
| | Traes_4BS_3BCDF0612.1 | Gene: SEQ ID NO: 288 |
| | (wheat homolog to SEQ ID | Protein: SEQ ID NO: 541 |
| | NO: 282) | |
| 59 | Traes_5BL_AAC9C7238.2 | Gene: SEQ ID NO: 283 |
| | | Protein: SEQ ID NO: 538 |
| 60 | Traes_2BL_C313CAB22.2 | Gene: SEQ ID NO: 284 |
| | | Protein: SEQ ID NO: 539 |

TABLE 15
dTF22 ortholog in Camelina sativa

| | |
|---|---|
| Switchgrass | Camelina |

| dTF | Gene | SEQ ID |
|---|---|---|
| 22 | Csa18g040020 | Gene: SEQ ID NO: 285 |
| | | Protein: SEQ ID NO: 542 |

### Example 7. Multiplex Editing of More than One Transcription Factor

Multiple dTFs can be edited to reduce or eliminate their activity in plants. The Venn diagram in FIG. 1 illustrates combinations of dTFs that are downregulated by more than one of the global regulatory genes STR1, STF1, and BMY1 using a cutoff of greater than 2 fold (log 2<−1). These include dTFs 1, 2, 3, 7, 9, 10, 18, and 22 (Table 1). Multiplex editing constructs can be produced to edit all dTFs selected from the group of dTF 1, 2, 3, 7, 9, 10, 18, and 22, or several select members of the group. We have found with our work in Camelina that multiplex editing will produce lines with all of the targets edited, as well as a range of lines with only some of the targets edited. Thus a multiplex editing vector can be designed to edit for example five of the dTFs and lines with combinations of less than five edits will typically also be isolated and can be tested.

While dTF22 is the only dTF that is downregulated by all three of the global regulatory genes STR1, STF1, and BMY1 by greater than 2 fold (log 2<−1), there are several dTFs that are downregulated by all three global regulatory genes by less than 2 fold, including dTFs 3, 9, 10, 14, 17, 18, and 60. Multiplex editing constructs can be constructed to simultaneously edit dTFs 3, 9, 10, 14, 17, 18, 22, and 60 or several dTFs selected from dTFs 3, 9, 10, 14, 17, 18, 22, and 60.

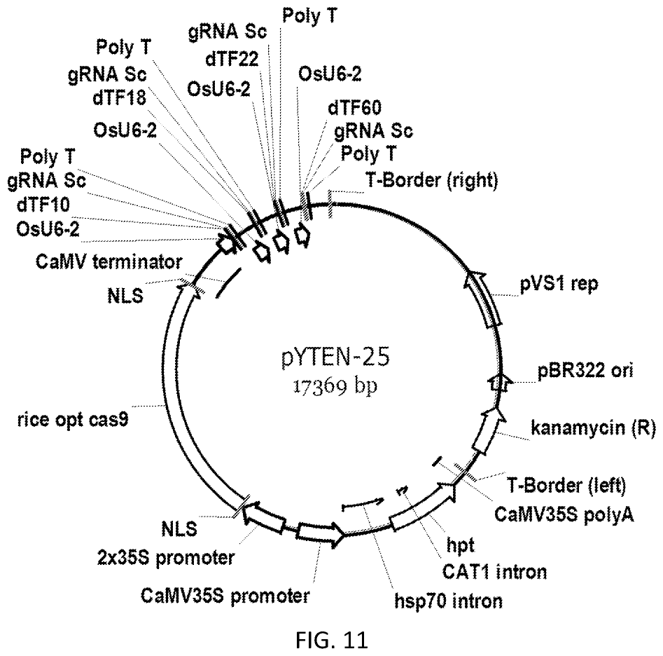

Multiplex editing of more than one dTF is illustrated using construct pYTEN-25 (FIG. 11, SEQ ID NO: 286) targeted to maize orthologs of dTFs 10, 18, 22 and 60 using the guide target sequence #4 for dTF10, dTF18, and dTF22 described in Table 9 and a guide target sequence of 5′-CTGAAGCCGAACCAGCCTGG-3′ (SEQ ID NO: 697) for dTF60.

Other useful gene combinations for editing include:

(a) dTF22 in combination with one more dTFs selected from dTFs 1-21 and dTFs 59-60;

(b) One or more of the dTFs selected from dTF1, dTF3, and dTF7, which are the three dTFs down regulated by more than 2 fold by both STR1 and BMY1 (FIG. 1, Table 1);

(c) dTF22 in combination with one or more of the dTFs selected from dTF1, dTF3, and dTF7;

(d) One or more of the dTFs selected from dTF2, dTF9, dTF10, and dTF18 which are the four dTFs down regulated by more than 2 fold by both STF1 and BMY1 (FIG. 1, Table 1); and

(e) dTF22 in combination with one or more of the dTFs selected from dTF2, dTF9, dTF10, and dTF18.

Genetic constructs for multiplex editing of these combinations can be constructed in a similar fashion as pYTEN-25 by switching the guide target sequences to match the genes to be targeted for editing.

Editing of dTF1, dTF2, and/or dTF7 can also be performed alone or in combination with dTF22. Example guide target sequences for editing of these downstream transcription factors in rice are shown in Table 16.

Useful gene combinations for editing in rice include:

(a) editing dTF1, 2, or 7 individually;

(b) multiplex editing of dTF22 and dTF1;

(c) multiplex editing of dTF22 and dTF2;

(d) multiplex editing of dTF22 and dTF7;

(e) multiplex editing of dTF1, 2, and 7;

(f) multiplex editing of dTF22, dTF1, dTF2, and dTF7;

(g) multiplex editing of dTF1 and dTF2;

(h) multiplex editing of dTF22, dTF1, and dTF2;

(i) multiplex editing of dTF1 and dTF7;

(j) multiplex editing of dTF22, dTF1 and dTF7;

(k) multiplex editing of dTF2 and dTF7; and

(l) multiplex editing of dTF22, dTF2, and dTF7.

TABLE 16
Guide target sequences for Cas9 mediated genome
editing of coding sequences of transcription
factor genes in rice

| | | Guide target | | |
|---|---|---|---|---|
| | | | Guide target | |
| | | | sequence | |
| Gene | Rice ortholog | Strand² | (5′ to 3′) | PAM³ |
| dTF1 | LOC_Os0lg47370 | + | CAACAGCAACAGTGTCA | TGG |
| | (SEQ ID NO: | | ACA (SEQ ID NO: | |
| | 189) | | 699) | |
| dTF2 | LOC_Os05g49240 | + | GAAGGAGAACAAGATG | AGG |
| | (SEQ ID NO: | | TTCG (SEQ ID NO: | |
| | 190) | | 700) | |
| dTF7 | LOC_Os12g40920 | − | TTTGATGTACCACTATT | TGG |
| | (SEQ ED NO: | | AGC (SEQ ID NO: | |
| | 195) | | 701) | |
| dTF22 | LOC_Os03g41330 | | TGCTGCGGCCGAGCATC | TGG |
| | (SEQ ID NO: | | GAG (SEQ ID NO: | |
| | 27) | | 554) | |

¹Sequence deposited under SEQ ID contains only the coding sequence of the gene.

### Example 8. CRISPR Editing with the CpfI Nuclease

In some cases, it may be desirable to use a nuclease with a different PAM sequence than the Cas9 enzyme. The CpfI class of enzymes have a different PAM sequence, depending on their source, allowing cuts at different genomic sequences than Cas9, which has a PAM sequence of “NGG”. There are several CpfI enzymes available (Zetsche et al., 2015, Cell, 163, 759; Gao et al., 2017, Nature Biotech., doi:10.1038/nbt.3900; Tang et al., 2017, Nat Plants, 3, Article number 17018; Wang et al., Molecular Plant, 2017, 10, 1011), some which are listed in Table 17 with their corresponding PAM sequences, all of which are useful for practicing this invention. Table 18 contains guide target sequences to illustrate the editing of the CDS of dTF22 using either the AsCpfI or the AsCpfI variant K607R enzymes.

TABLE 17
Cpfl enzymes and their variants
useful for genome editing

| Cpfl Enzyme | Source | PAM¹ |
|---|---|---|
| AsCpfl | Acidaminococcus sp. | TTTV |
| | BV3L6 | |
| AsCpfl S542R/K607R | AsCpfl variant | TYCV |
| AsCpfl S542R/K548V/N552R | AsCpfl variant | TATV |
| LbCpfl | Lachnospiraceae | TTTV |
| | bacterium ND2006 | |
| LbCpfl G532R/K595R | LbCpfl variant | TYCV |
| FnCpfl | Francisella novicida | TTN |
| | U112 (NC_008601) | |

¹Abbreviations in PAM consensus sequences; Y = C or T; V = A, C or G; N = any base

TABLE 18
Guide target sequences for Cpfl mediated editing of plant dTF22 genes

| | Locus name for dTF22 | | Target | | Guide target sequence |
|---|---|---|---|---|---|
| Plant species | ortholog¹ | CpfI Enzyme | strand² | PAM³ | (5′ to 3′) |
| Medicago truncatula⁴ | Medtr1g106420 | AsCpfl | + | TTTT | GAGAAAGGGATGCAGTG |
| | (SEQ ID NO: 33) | | | | AGGATT (SEQ ID NO: 702) |
| Camelina (Camelina sativa) | Csa18g040020 | AsCpfl | − | TTTC | AATCCATTGAATACATG |
| | (SEQ ID NO: 285) | | | | GTCGGA (SEQ ID NO: 703) |
| Canola (B. napus cv. Darmor-bzh) | BnaCO2g16720D | AsCpfl | + | TTTC | ATCTCCGCCGTACCGGA |
| | (SEQ ID NO: 186) | | | | ATCTCA (SEQ ID NO: 704) |
| Maize (Zea mays) | GRMZM2G017319 | AsCpfl | − | TCCG | CCCCAGCATCGACTGGA |
| | (SEQ ID NO: 28) | variant K607R | | | TCCACG (SEQ ID NO: 705) |
| Rice (Oryza sativa) | LOC_Os03g41330 | AsCpfl | − | TTTA | TCGTCCGCCCGCACGCCT |
| | (SEQ ID NO: 210) | | | | CGTAC (SEQ ID NO: 706) |
| Sorghum (Sorghum bicolor) | Sb0lg014800 | AsCpfl | − | TTTC | TGGACGGCGGCGCGGAG |
| | (SEQ ID NO: 258) | | | | GAGGAG (SEQ ID NO: 707) |
| Soybean (Glycine max) | Glyma05g22120 | AsCpfl | − | TTTC | GATCCACTGCAAACAAG |
| | (SEQ ID NO: 162) | | | | GGCGCA (SEQ ID NO: 708) |
| Wheat (Triticum aestivum) | Traes_4AL_7241716B6 | AsCpfl | + | TTCG | TCGCCAAGTTCTTCGGCC |
| | (SEQ ID NO: 34) | variant K607R | | | GCGCC (SEQ ID NO: 709) |

¹For maize, Medicago truncatula, and wheat, the SEQ ID includes the 5′ UTR region upstream of the ATG (predicted by Phytozome, MaizeGDB, GenBank, and/or transcript analysis), 1000 bp of promoter sequence upstream of the 5′ UTR, as well as the coding sequence of the gene including any introns. For soybean, Brassica napus, Camelina sativa, rice, and sorghum bicolor, the SEQ ID includes only the coding sequence of the gene.
²Strand (+/−) refers to the gRNA binding to either the forward strand of DNA (+) or its reverse complement (−).
³PAM refers to the protospacer adjacent motif for the CAS9 enzyme that resides directly adjacent to the 3′ end of the guide RNA target.
⁴The genome sequence for alfalfa (Medicago sativa) is not publically available. The sequence of a close relative to alfalfa, Medicago truncatula was used to find the dTF22 ortholog. Once the genome sequence of alfalfa (Medicago sativa) is publically available, the dTF22 ortholog can be found by comparison with the Medicago truncatula and switchgrass genes.

CpfI mediated genome editing can be performed as follows. Constructs are produced that contain the following expression cassettes: (a) an expression cassette for the CpfI gene that contains a promoter functional in that crop, the CpfI gene that includes nuclear localization sequences on the 5′ and 3′ end of the gene, and a terminator; (b) one or more expression cassettes for CRISPR RNAs that consists of a promoter, a gRNA that consists of 19 nucleotide repeat and a guide target sequence with about 23-25 bp homology downstream of a PAM sequence specific for the CpfI enzyme used (Fagerlund, R. D. 2015, Genome Biology, 16, 251); and a poly T-termination sequence. The promoter for gRNAs is preferably a U6 promoter functional in the crop to be transformed; and (c) an expression cassette for a selectable marker that can be used for the specific crop for selection of transformants. For Agrobacterium-mediated transformation, these expression cassettes can be cloned into one or more binary vectors for transformation of the appropriate explant of the crop. For stable transformation by particle bombardment or protoplast transformation, expression cassettes can be introduced as a DNA fragment(s) or can be localized on one or more simple plasmid vectors. For both methods, plants can be screened for edits using Next Generation Sequencing methods. After the edits are obtained, the expression cassettes described above can be removed by segregation using conventional breeding methods for the crop.

For transient expression in protoplasts, the expression cassettes described above for the CpfI and the gRNA can be introduced as one or more DNA fragments or can be localized on one or more simple vectors. An expression cassette for a selectable marker is not required. Protoplast cultures or alternatively, callus cultures derived from the protoplast cultures, can be screened for edits using Next Generation Sequencing methods, and protoplast or callus cultures with the edits can be regenerated into plants.

RNPs can also be used with the CpfI enzyme to perform DNA free genome editing in protoplasts (Kim, H. et al., 2016, Nature Communications, DOI: 10:1038/ncomms14406). For editing using RNPs, purified CpfI enzyme can be mixed with one or more gRNAs to form a complex of the CpfI enzyme and the gRNAs which can then be introduced directly to protoplasts. Protoplast cultures or alternatively, callus cultures derived from the protoplast cultures, can be screened for edits using Next Generation Sequencing methods, and protoplast or callus cultures with the edits can be regenerated into plants.

The ability of the CpfI enzyme to cleave its own CRISPR RNA also allows an array of sgRNAs to be arranged on a single genetic fragment which is subsequently cleaved by CpfI to initiate multiplex editing (Zetsche et al., 2017, Nature Biotech, 35, 31-34).

Reference to a “Sequence Listing,” a Table, or a Computer Program Listing Appendix Submitted as an ASCII Text File

The material in the ASCII text file, named “YTEN-59975WO-sequence-listing_ST25.txt”, created Oct. 30, 2018, file size of 1,409,024 bytes, is hereby incorporated by reference.

1. A method for modifying a plant, the method comprising downregulating expression, in a plant, of at least one polynucleotide sequence encoding a transcription factor that comprises a sequence-specific DNA binding domain with homology to proteins in the LBD family and has at least 30% sequence identity to SEQ ID NO: 310, thereby modifying the plant.

2-7. (canceled)

8. The method of claim 1, wherein the at least one polynucleotide sequence that is downregulated exhibits at least a two-fold change in expression as compared to that of a control plant.

9. The method of claim 8, wherein the at least one polynucleotide sequence that is downregulated has been downregulated by overexpression of one or more global transcription factors selected from STR1, BMY, or STIF1.

10. The method of claim 1, wherein the at least one polynucleotide sequence that is downregulated has been downregulated by one or more of gene inactivation, deletion, insertion and/or substitution of one or more nucleotides, site-specific mutagenesis, chemical mutagenesis, targeting induced local lesions in genomes (TILLING), knock-out techniques, gene editing techniques using CRISPR nuclease selected from Cas nuclease, Cas9 nuclease, CasX nuclease, CasY nuclease, a Cpf1 nuclease, a C2c1 nuclease, a C2c2 nuclease (Cas13a nuclease), or a C2c3 nuclease, NgAgo nuclease, TALEN or ZFN techniques, or gene silencing induced by RNA interference.

11. (canceled)

12. (canceled)

13. The method of claim 1, wherein the modified plant is soybean, canola, Medicago truncatula, alfalfa, sorghum, rice, wheat or Camelina.

14. The method of claim 1, wherein the modified plant exhibits one or more enhanced characteristics selected from higher photosynthesis rates, reduced photorespiration rates, higher biomass yield or content, higher seed yield, improved harvest index, higher seed oil content, improved nutritional composition, improved nitrogen use efficiency, drought resistance, flood resistance, disease resistance, faster seed germination and plant emergence, improved seedling vigor, salt tolerance, higher CO₂ assimilation rate, or lower transpiration rate.

15. The method of claim 14, wherein the modified plant exhibits an increase in seed oil content or seed yield as compared to a control plant.

16. The method of claim 15, wherein the seed oil content or seed yield of the modified plant is increased by 10%, 20%, 30%, 40%, 50%, 60%, 70%, 80%, 90%, 100% or higher relative to a control plant.

17-28. (canceled)

29. A DNA construct comprising:
(a) an expression cassette containing a polynucleotide sequence encoding a CRISPR nuclease;
(b) DNA encoding at least one guide RNA targeting the 5′ upstream region, promoter, terminator or coding sequence of a polynucleotide sequence encoding a transcription factor that comprises a sequence-specific DNA binding domain with homology to proteins in the LBD family and has at least 30% sequence identity to SEQ ID NO: 310; and
(c) an expression cassette for a selectable marker.

30. The DNA construct of claim 29, wherein the DNA encoding the at least one guide RNA is capable of downregulating the polynucleotide sequence encoding a transcription factor that comprises a sequence-specific DNA binding domain with homology to proteins in the LBD family and has at least 30% sequence identity to SEQ ID NO: 310, thereby producing enhanced characteristics in a plant selected from higher photosynthesis rates, reduced photorespiration rates, higher biomass yield or content, higher seed yield, improved harvest index, higher seed oil content, improved nutritional composition, improved nitrogen use efficiency, drought resistance, flood resistance, disease resistance, faster seed germination and plant emergence, improved seedling vigor, salt tolerance, higher CO₂ assimilation rate, or lower transpiration rate.

31. A modified plant comprising the DNA construct of claim 29.

32-39. (canceled)

40. A method of modifying a plant cell comprising:
(a) expressing one or more site-specific nucleases in a plant cell, wherein the one or more nucleases target and cleave chromosomal DNA of one or more endogenous genes, their promoters, their 5′UTRs, and/or their polyadenylation sequences, and wherein the one or more endogenous genes comprise a polynucleotide sequence encoding a transcription factor that comprises a sequence-specific DNA binding domain with homology to proteins in the LBD family and has at least 30% sequence identity to SEQ ID NO: 310;
(b) integrating one or more exogenous sequences into the one or more endogenous genes, their promoters, their 5′UTRs, and/or their polyadenylation sequences within the genome of the plant cell, wherein the one or more endogenous genes, their promoters, their 5′UTRs, and/or their polyadenylation sequences are modified such that the one or more endogenous genes do not express their corresponding endogenous gene product(s); and
(c) selecting modified plant cells that exhibit enhanced characteristics from among the plant cells in which the one or more exogenous sequences have been integrated.

41. The method of claim 40, wherein the one or more exogenous sequences are selected from a donor polynucleotide, a transgene, or a combination thereof.

42. The method of claim 40, wherein the one or more exogenous sequences encode a transgene and/or are expressed to produce an RNA molecule.

43. The method of claim 40, wherein the one or more exogenous sequences comprise a multiplex of gene edits made in the one or more endogenous genes, their promoters, their 5′UTRs, and/or their polyadenylation sequences.

44. (canceled)

45. (canceled)

46. The method of claim 40, wherein the one or more endogenous genes is downregulated.

47. (canceled)

48. (canceled)

49. The method of claim 40, wherein the modified plant cell is a cell of soybean, canola, Medicago truncatula, alfalfa, sorghum, rice, wheat or Camelina.

50-52. (canceled)

53. The method for modifying a plant according to claim 1, wherein the transcription factor that comprises a sequence-specific DNA binding domain with homology to proteins in the LBD family comprises one or more of SEQ ID NOs: 310, 334, 354, 371, 387, 417, 441, 465, 489, 513, 537, 540, 541, 542, 546, or 761.

54. The method for modifying a plant according to claim 1, wherein the at least one polynucleotide sequence has at least 30% sequence identity to one or more of SEQ ID NOs: 22, 56, 76, 93, 109, 162, 186, 210, 234, 258, 282, 285, 287, 288, 544, or 741.

55. The method for modifying a plant according to claim 54, wherein the at least one polynucleotide sequence comprises one or more of SEQ ID NOs: 22, 56, 76, 93, 109, 162, 186, 210, 234, 258, 282, 285, 544, or 741.
